(19) United States
(12) Patent Application Publication
(10) Pub. No.: US 2018/0050993 A1
(43) Pub. Date: Feb. 22, 2018

(54) NOVEL FLUORINATED DERIVATIVES AS EGFR INHIBITORS USEFUL FOR TREATING CANCERS

(71) Applicant: Trillium Therapeutics Inc., Mississauga (CA)

(72) Inventors: Abdelmalik Slassi, Mississauga (CA); Peter Dove, Burlington (CA)

(73) Assignee: Trillium Therapeutics Inc., Mississauga, ON (CA)

(21) Appl. No.: 15/546,309

(22) PCT Filed: Feb. 3, 2016
(86) PCT No.: PCT/CA2016/050094

§ 371 Date: Jul. 26, 2017

Related U.S. Application Data
(60) Provisional application No. 62/111,240, filed on Feb. 3, 2015.

(51) Int. Cl. C07D 239/94; C07D 401/12; C07D 403/12; C07C 22/08; C07C 25/13

(52) U.S. Cl. CPC C07D 239/94; C07D 401/12; A61K 31/517; C07C 22/08; C07C 25/13; C07D 403/12

(57) ABSTRACT

A novel class of fluorinated derivatives of Formula I have been prepared and found to be useful in the treatment of cancers and other EGFR related disorders.

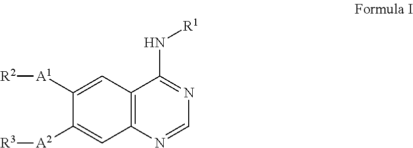

### CROSS-REFERENCE TO RELATED APPLICATIONS

The present application claims the benefit of priority from co-pending U.S. provisional patent application Ser. No. 62/111,240 filed on Feb. 3, 2015, the contents of which are incorporated herein by reference.

### FIELD

The present application relates to novel fluorinated derivatives, to processes for their preparation, to compositions comprising them, and to their use in therapy. More particularly, it relates to compounds useful in the treatment of diseases, disorders or conditions mediated by epidermal growth factor receptor. Such compounds and salts thereof may be useful in the treatment or prevention of a number of different cancers. The application also relates to pharmaceutical compositions comprising said compounds and salts thereof, especially useful polymorphic forms of these compounds and salts, intermediates useful in the manufacture of said compounds and to methods of treatment of diseases mediated by various different forms of EGFR using said compounds and salts thereof.

### BACKGROUND

Epidermal Growth Factor Receptor (EGFR) is a transmembrane protein tyrosine kinase of the ErbB receptor family. Upon binding of a growth factor ligand such as epidermal growth factor (EGF), the receptor can homo-dimerise with another EGFR molecule or hetero-dimerise with another family member such as ErbB2 (HER2), ErbB3 (HER3), or ErbB4 (HER4). Homo- and/or hetero-dimerisation of ErbB receptors results in the phosphorylation of key tyrosine residues in the intracellular domain and leads to the stimulation of numerous intracellular signal transduction pathways involved in cell proliferation and survival. Deregulation of ErbB family signalling promotes proliferation, invasion, metastasis, angiogenesis, and tumour cell survival and has been described in many human cancers, including those of the lung, head, neck and breast. The ErbB family therefore represents a rational target for anticancer drug development and a number of agents targeting EGFR or ErbB2 are now clinically available, including gefitinib (IRESSA™), erlotinib (TARCEVA™) and lapatinib (TYKERB™, TYVERB™). Detailed reviews of ErbB receptor signalling and its involvement in tumourigenesis are provided in New England Journal of Medicine (2008) Vol. 358, 1160-74 and Biochemical and Biophysical Research Communications (2004) Vol. 319, 1-11. In 2004 it was reported (Science [2004] Vo1.304, 1497-500 and New England Journal of Medicine [2004] Vol. 350, 2129-39) that activating mutations in EGFR correlated with response to gefitinib therapy in non-small-cell lung cancer (NSCLC).

The most common EGFR activating mutations, L858R and de1E746_A750, result in an increase in affinity for small molecule tyrosine kinase inhibitors such as gefitinib and erlotinib and a decrease in affinity for adenosine triphosphate (ATP) relative to wild type (WT) EGFR. Ultimately, acquired resistance to therapy with gefitinib or erlotinib arises, for example by mutation of the gatekeeper residue T790M, which is reportedly detected in 50% of clinically resistant patients. This mutation is not believed to hinder the binding of gefitinib or erlotinib to EGFR sterically, it merely alters the affinity to ATP to levels comparable to WT EGFR. In view of the importance of this mutation in resistance to existing therapies targeting EGFR, agents which inhibit EGFR harbouring the gatekeeper mutation may be especially useful in the treatment of cancer. There remains a need for compounds that exhibit favourable potency against WT EGFR versus activating mutant forms of EGFR (for example the L858R EGFR mutant, or the de1E746_A750 mutant or the Exon19 deletion EGFR mutant) and/or resistant mutant forms of EGFR (for example T790M EGFR mutant), and/or selectivity over other enzyme receptors. In this regard, there remains a need for compounds that show a higher inhibition of certain activating or resistance mutant forms of EGFR while at the same time showing relatively low inhibition of WT EGFR. Such compounds may be expected to be more suitable as therapeutic agents, particularly for the treatment of cancer, due to the reduction in toxicity associated with WT EGFR inhibition. Such toxicity is known to manifest themselves in humans as skin rashes and/or diarrhoea. The applicants have surprisingly found that one or more fluorine derived compounds have high potency against of EGFR.

Glioblastoma multiforme (GBM) is the most aggressive of the astrocytic malignancies and the most common intracranial tumor in adults. Although the EGFR is overexpressed and/or mutated in at least 50% of GBM cases and is required for tumor maintenance in animal models, EGFR inhibitors have thus far failed to deliver significant responses in GBM patients. One inherent resistance mechanism in GBM is the coactivation of multiple receptor tyrosine kinases, which generates redundancy in activation of phosphoinositide-3′-kinase (PI3K) signaling. Phosphatase and tensin homolog deleted on chromosome 10 (PTEN) tumor suppressor is frequently phosphorylated at a conserved tyrosine residue, Y240, in GBM clinical samples. Phosphorylation of Y240 is associated with shortened overall survival and resistance to EGFR inhibitor therapy in GBM patients and plays an active role in mediating resistance to EGFR inhibition in vitro. Y240 phosphorylation can be mediated by both fibroblast growth factor receptors and SRC family kinases (SFKs) but does not affect the ability of PTEN to antagonize PI3K signaling. These findings show that, in addition to genetic loss and mutation of PTEN, its modulation by tyrosine phosphorylation has important implications for the development and treatment of GBM.

Fluorine has found interest in bioorganic and structural chemistry over the past decade and has become a useful feature in drug design. The small and highly electronegative fluorine atom can play a useful role in medicinal chemistry. Selective installation of fluorine into a therapeutic or diagnostic small molecule candidate can give a number of useful pharmacokinetic and/or physicochemical properties such as improved metabolic stability and enhanced membrane permeation. Increased binding affinity of fluorinated drug candidates to a target protein has also been documented in a some of cases. A further emerging application of the fluorine atom is the use of the ¹⁸F isotope as a radiolabel tracer atom in the sensitive technique of Positron Emission Tomography (PET) imaging.

Fluorine substitution has been investigated in drug research as a means of enhancing biological activity and/or increasing chemical and/or metabolic stability. Factors to be considered when synthesising fluorine-containing compounds include (a) the relatively small size of the fluorine atom (van der Waals radius of 1.47 Å), comparable to hydrogen (van der Waals radius of 1.20 Å), (b) the highly electron-withdrawing nature of fluorine, (c) the greater stability of the C—F bond compared to the C—H bond and (d) the greater lipophilicity of fluorine compared to hydrogen.

Despite the fact that fluorine is slightly larger than hydrogen, several studies have shown that the fluorine atom is a reasonable hydrogen mimic with minimal steric perturbations with respect to the compound's mode of binding to a receptor or enzyme [Annu. Rev. Pharmacol. Toxicol. 2001, 41, 443-470]. However, the introduction of a fluorine atom can significantly alter the physicochemical properties of a compound due to its high electronegativity. Therefore this type of modification can induce altered biological responses of the molecule.

### SUMMARY

A novel class of fluorinated derivatives of Formula I has been prepared and found to be useful in the treatment of cancers and other EGFR related disorders.

The compound(s) of the application also exhibit advantageous physical properties (for example higher permeability, enhanced CNS penetration and/or lower plasma protein binding) and/or favourable toxicity profiles (for example a decreased hERG blocking liability) and/or favourable metabolic profiles in comparison with other known EGFR/EGFR-mutant inhibitors. Therefore, in some embodiments, the compounds of the application are especially useful in the treatment of disease states in which EGFR and/or activating mutations of EGFR and/or resistance mutations of EGFR are implicated, for example in the treatment of cancer.

Accordingly, the present application includes a compound of Formula I or a pharmaceutically acceptable salt, solvate or prodrug thereof:

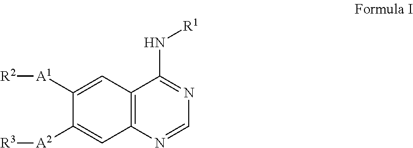

wherein:
R¹ is selected from unsubstituted or substituted aryl and unsubstituted or substituted heteroaryl, wherein the substituents for R¹ are selected from one or more of halogen, C₁₋₆alkyl, haloC₁₋₆alkyl, CN, C(O)R⁴, OR⁴, SR⁴, NR⁴R⁵, C(O)OR⁴, C(O)NR⁴R⁵, S(O)R⁴, SO₂R⁴, OC(O)R⁴, OC(O)OR⁴, OC(O)NR⁴R⁵, OC(S)NR⁴R⁵, OS(O)R⁴, OSO₂R⁴, NR⁴(OR⁵), NR⁶C(O)NR⁴R⁵, NR⁶C(S)NR⁴R⁵, NR⁵C(O)OR⁴, NR⁵C(S)OR⁴, NR⁵C(O)R⁴, C₁₋₆alkyleneC(O)R⁴, C₁₋₆alkyleneOR⁴, C₁₋₆alkyleneSR⁴, C₁₋₆alkyleneNR⁴R⁵, C₁₋₆alkyleneC(O)OR⁴, C₁₋₆alkyleneC(O)NR⁴R⁵, C₁₋₆alkyleneS(O)R⁴, C₁₋₆alkyleneSO₂R⁴, C₁₋₆alkyleneOC(O)R⁴, C₁₋₆alkyleneOC(O)OR⁴, C₁₋₆alkyleneOC(O)NR⁴R⁵, C₁₋₆alkyleneOC(S)NR⁴R⁵, C₁₋₆alkyleneOS(O)R⁴, C₁₋₆alkyleneOSO₂R⁴, C₁₋₆alkyleneNR⁴(OR⁵), C₁₋₆alkyleneNR⁶C(O)NR⁴R⁵, C₁₋₆alkyleneNR⁶C(S)NR⁴R⁵, C₁₋₆alkyleneNR⁵C(O)OR⁴, C₁₋₆alkyleneNR⁵C(S)OR⁴, C₁₋₆alkyleneNR⁵C(O)R⁴, C₂₋₆alkynyl, C₂₋₆alkynyleneC(O)R⁴, C₂₋₆alkynyleneOR⁴, C₂₋₆alkynyleneSR⁴, C₂₋₆alkynyleneNR⁴R⁵, C₂₋₆alkynyleneC(O)OR⁴, C₂₋₆alkynyleneC(O)NR⁴R⁵, C₂₋₆alkynyleneS(O)R⁴, C₂₋₆alkynyleneSO₂R⁴, C₂₋₆alkynyleneOC(O)R⁴, C₂₋₆alkynyleneOC(O)OR⁴, C₂₋₆alkynyleneOC(O)NR⁴R⁵, C₂₋₆alkynyleneOC(S)NR⁴R⁵, C₂₋₆alkynyleneOS(O)R⁴, C₂₋₆alkynyleneOSO₂R⁴, C₂₋₆alkynyleneNR⁴(OR⁵), C₂₋₆alkynyleneNR⁶C(O)NR⁴R⁵, C₂₋₆alkynyleneNR⁶C(S)NR⁴R⁵, C₂₋₆alkynyleneNR⁵C(O)OR⁴, C₂₋₆alkynyleneNR⁵C(S)OR⁴, C₂₋₆alkynyleneNR⁵C(O)R⁴ and 3-7 membered heterocycloalkyl;
R² and R³ are independently selected from C₁₋₂₀alkyl, C₆₋₂₀aryl, heteroaryl, C₃₋₂₀cycloalkyl, heterocycloalkyl, C₁₋₁₀alkyleneC₆₋₂₀aryl, C₁₋₁₀alkyleneheteroaryl, C₁₋₁₀alkyleneC₃₋₂₀cycloalkyl, C₁₋₁₀alkyleneheterocycloalkyl, C(O)C₁₋₂₀alkyl, C(O)C₆₋₂₀aryl, C(O)heteroaryl, C(O)C₃₋₂₀cycloalkyl, C(O)NR⁶heterocycloalkyl, C(O)NR⁶C₁₋₂₀alkyl, C(O)NR⁶C₆₋₂₀aryl, C(O)NR⁶heteroaryl, C(O)NR⁶C₃₋₂₀cycloalkyl and C(O)NR⁶heterocycloalkyl, wherein R² and R³ are unsubstituted or substituted with one or more substituents independently selected from halo, C₁₋₆alkyl, OC₁₋₆alkyl, halo-substituted C₁₋₆alkyl, halo-substituted OC₁₋₆alkyl, halo-substituted SC₁₋₆alkyl halo-substituted C₁₋₆alkyleneOC₁₋₆alkyl, halo-substituted C₁₋₆alkyleneSC₁₋₆alkyl, halo-substituted C₁₋₆alkyleneS(O)C₁₋₆alkyl, halo-substituted C₁₋₆alkyleneSO₂C₁₋₆alkyl and C₁₋₆alkyleneOhalo-substituted C₁₋₆alkyl, provided that at least one of R² and R³ comprises at least one fluorine atom;
R⁴, R⁵ and R⁶ are independently selected from H, C₆₋₁₀aryl, heteroaryl, C₃₋₁₀cycloalkyl, C₃₋₁₀heterocycloalkyl, haloC₁₋₆alkyl and C₁₋₆alkyl; and
A¹ and A² are independently selected from CH₂, O, S, S(O), SO₂NH and NR⁵.

The present application also includes a composition comprising one or more compounds of the application and a carrier. In an embodiment, the composition is a pharmaceutical composition comprising one or more compounds of the application and a pharmaceutically acceptable carrier.

The compounds of the application have been shown to be capable of inhibiting EGFR protein function. Therefore the compounds of the application are useful for treating diseases, disorders or conditions treatable by inhibition of EGFR. Accordingly, the present application also includes a method of treating a disease, disorder or condition treatable by inhibition of EGFR, comprising administering a therapeutically effective amount of one or more compounds of the application to a subject in need thereof.

In a further embodiment, the compounds of the application are used as medicaments. Accordingly, the application also includes a compound of the application for use as a medicament.

The present application also includes a use of one or more compounds of the application for treatment of a disease, disorder or condition by inhibition of EGFR as well as a use of one or more compounds of the application for the preparation of a medicament for treatment of a disease, disorder or condition by inhibition of EGFR. The application further includes one or more compounds of the application for use in treating a disease, disorder or condition treatable by inhibition of EGFR.

In an embodiment, the disease, disorder or condition treatable by inhibition of EGFR is a neoplastic disorder. In an embodiment, the treatment is in an amount effective to ameliorate at least one symptom of the neoplastic disorder, for example, reduced cell proliferation or reduced tumor mass in a subject in need of such treatment.

In an embodiment, the disease, disorder or condition is cancer.

In an embodiment, the disease, disorder or condition is a disease, disorder or condition associated with an uncontrolled and/or abnormal cellular activity affected directly or indirectly by EGFR. In another embodiment, the uncontrolled and/or abnormal cellular activity that is affected directly or indirectly by EGFR is proliferative activity in a cell.

The application also includes a method of inhibiting proliferative activity in a cell, comprising administering an effective amount of one or more compounds of the application to the cell.

In a further embodiment the EGFR-mediated disease, disorder or condition is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

The application additionally provides a process for the preparation of compounds of the application. General and specific processes are discussed in more detail below and set forth in the Examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows the maximum peak concentrations in the brain of erlotinib compared to exemplary compounds 2A.HCl and 2D.HCl 4 hours post administration in a 50 mg/kg rat (PO administration).

FIG. 2 shows the binding affinity values (K_{d}) of exemplary compounds 2A.HCl and 2D.HCl for the ephrin receptor kinase, EPHA6.

### DETAILED DESCRIPTION

### Definitions

Unless otherwise indicated, the definitions and embodiments described in this and other sections are intended to be applicable to all embodiments and aspects of the application herein described for which they are suitable as would be understood by a person skilled in the art. Unless otherwise specified within this application or unless a person skilled in the art would understand otherwise, the nomenclature used in this application generally follows the examples and rules stated, for example, in “Nomenclature of Organic Chemistry” (Pergamon Press, 1979), Sections A, B, C, D, E, F, and H. Optionally, a name of a compound may be generated using a chemical naming program such as ACD/ChemSketch, Version 5.09/September 2001, Advanced Chemistry Development, Inc., Toronto, Canada.

The term “compound of the application” or “compound of the present application” and the like as used herein refers to a compound of Formula I, and pharmaceutically acceptable salts, solvates, prodrugs and/or radiolabeled versions thereof.

The term “composition of the application” or “composition of the present application” and the like as used herein refers to a composition, such as a pharmaceutical composition, comprising one or more compounds of Formula I, or pharmaceutically acceptable salts, solvates, prodrugs and/or radiolabeled versions thereof.

The term “and/or” as used herein means that the listed items are present, or used, individually or in combination. In effect, this term means that “at least one of” or “one or more” of the listed items is used or present. The term “and/or” with respect to pharmaceutically acceptable salts, solvates and/or prodrugs thereof means that the compounds of the application exist as individual salts, hydrates or prodrugs, as well as a combination of, for example, a salt of a solvate of a compound of the application or a salt of a prodrug of a compound of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or openended terms and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of” as used herein is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The expression “proceed to a sufficient extent” as used herein with reference to the reactions or process steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms.

The term “alkylene”, whether it is used alone or as apart of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene groups are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain unsaturated alkyl groups containing at least one triple bond. The number of carbon atoms that are possible in the referenced alkylyne group are indicated by the prefix “Cₙ₁₋ₙ₂”. For example, the term C₂₋₆alkynyl means an alkynyl group having 2, 3, 4, 5 or 6 carbon atoms and at least one triple bond.

The term “haloalkyl” as used herein refers to an alkyl group wherein one or more, including all of the hydrogen atoms are replaced by a halogen atom. In an embodiment, the halogen is fluorine, in which case the haloalkyl is referred to herein as a “fluoroalkyl” group. In another embodiment, the haloalkyl comprises at least one —CHF₂ group.

The term “alkoxy” as used herein, whether it is used alone or as part of another group, refers to the group “alkyl-O-” or “—O-alkyl”. The term C₁₋₁₀alkoxy means an alkyl group having 1, 2, 3, 4, 5, 6, 7, 8, 9 or 10 carbon atoms bonded to an oxygen atom. Exemplary alkoxy groups include without limitation methoxy, ethoxy, propoxy, isopropoxy, butoxy, t-butoxy and isobutoxy.

The term “cycloalkyl,” as used herein, whether it is used alone or as part of another group, means a saturated carbocyclic group containing a number of carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to cyclic groups containing from 6 to 20 carbon atoms and at least one aromatic ring. In an embodiment of the application, the aryl group contains from 6, 9 or 10 atoms, such as phenyl, naphthyl or indanyl.

The term “heterocycloalkyl” as used herein, whether it is used alone or as part of another group, refers to cyclic groups containing 3 to 20 atoms, suitably 3 to 10 atoms, and at least one non-aromatic ring in which one or more of the atoms are a heteromoiety selected from O, S, N, NH and NC₁₋₆alkyl. Heterocycloalkyl groups are either saturated or unsaturated (i.e. contain one or more double bonds) and contain one or more than one ring (i.e. are polycyclic). When a heterocycloalkyl group contains more than one ring, the rings may be fused, bridged, spirofused or linked by a bond. When a heterocycloalkyl group contains the prefix “Cₙ₁₋ₙ₂” this prefix indicates the number of carbon atoms in the corresponding carbocyclic group, in which one or more, suitably 1 to 5, of the ring atoms is replaced with a heteromoiety as defined above.

A first ring group being “fused” with a second ring group means the first ring and the second ring share at least two atoms there between.

The term “heteroaryl” as used herein refers to cyclic groups containing from 5 to 20 atoms, suitably 5 to 10 atoms, at least one aromatic ring and at least one a heteromoiety selected from O, S, N, NH and NC₁₋₆alkyl. Heteroaryl groups contain one or more than one ring (i.e. are polycyclic). When a heteroaryl group contains more than one ring, the rings may be fused, bridged, spirofused or linked by a bond. When a heteroaryl group contains the prefix “Cₙ₁₋ₙ₂” this prefix indicates the number of carbon atoms in the corresponding carbocyclic group, in which one or more, suitably 1 to 5, of the ring atoms is replaced with a heteromoiety as defined above.

A five-membered heteroaryl is a heteroaryl with a ring having five ring atoms, wherein 1, 2 or 3 ring atoms are a heteromoiety selected from O, S, N, NH and NC₁₋₆alkyl. Exemplary five-membered heteroaryls include but are not limited to thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered heteroaryl is a heteroaryl with a ring having six ring atoms wherein 1, 2 or 3 ring atoms are a heteromoiety selected from O, S, N, NH and NC₁₋₆alkyl. Exemplary six-membered heteroaryls include but are not limited to pyridnyl, pyrazinyl, pyrimidinyl, triazinyl and pyridazinyl.

As a prefix, the term “substituted” as used herein refers to a structure, molecule or group in which one or more available hydrogen atoms are replaced with one or more other chemical groups. In an embodiment, the chemical group is a C₁₋₄alkyl. In another embodiment, the chemical group is a C₁₋₁alkyl or a chemical group that contains one or more heteroatoms selected from N, O, S, F, Cl, Br, I, and P. Exemplary chemical groups containing one or more heteroatoms include heterocyclyl, —NO₂, —OR, —R′OR, —Cl, —Br, —I, —F, —CF₃, —C(═O)R, —NR₂, —SR, —SO₂R, —S(═O)R, —CN, —C(═O)OR, —C(═O)NR₂, —NRC(═O)R, —NRC(═O)OR, —R′NR₂, oxo (═O), imino (═NR), thio (═S), and oximino (═N—OR), wherein each “R” is hydrogen or a C₁₋₁₂alkyl and “R” is a C₁₋₁₂alkylene. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any available hydrogen on the phenyl ring.

As a suffix, the term “substituted” as used herein in relation to a first structure, molecule or group, followed by one or more variables or names of chemical groups, refers to a second structure, molecule or group that results from replacing one or more available hydrogens of the first structure, molecule or group with the one or more variables or named chemical groups. For example, a “phenyl substituted by nitro” refers to nitrophenyl.

The term “available”, as in “available hydrogen atoms” or “available atoms” refers to atoms that would be known to a person skilled in the art to be capable of replacement by a substituent.

The term “optionally substituted” refers to groups, structures, or molecules that are either unsubstituted or are substituted with one or more substituents.

The term “amine” or “amino,” as used herein, whether it is used alone or as part of another group, refers to radicals of the general formula —NRR′, wherein R and R′ are each independently selected from hydrogen or a alkyl group, such as C₁₋₆alkyl.

The terms “halo” or “halogen” as used herein, whether it is used alone or as part of another group, refers to a halogen atom and includes fluoro, chloro, bromo and iodo.

acac as used herein refers to acetylacetonate.

The term “atm” as used herein refers to atmosphere.

The term “aq.” as used herein refers to aqueous.

The terms “Boc” and “t-Boc” as used herein refer to the group tert-butoxycarbonyl.

DCM as used herein refers to dichloromethane.

DIPEA as used herein refers to N,N-Diisopropyl ethylamine.

DMF as used herein refers to dimethylformamide.

DMSO as used herein refers to dimethylsulfoxide.

EDCI.HCl as used herein refers to N-[3-(dimethylamino)propyl]-N′-ethylcarbodiimide hydrochloride.

EDC as used herein refers to 1-ethyl-3-(3-dimethylaminopropyl)carbodiimide.

Et₂O as used herein refers to diethylether.

EtOAc as used herein refers to ethyl acetate.

Et as used herein refers to the group ethyl.

Fmoc as used herein refers to the group 9-fluorenylmethyloxycarbonyl.

The term “hr(s)” as used herein refers to hour(s).

The term “min(s)” as used herein refers to minute(s).

HOBt as used herein refers to N-hydroxybenzotriazole.

HBTU as used herein refers to O-(Benzotriazol-1-yl)-N,N,N′,N′-tetramethyluronium hexafluorophosphate.

MeOH as used herein refers to methanol.

Me as used herein refers to the group methyl.

t-BuLi as used herein refers to tert-butyllithium.

ON as used herein refers to overnight.

RT as used herein refers to room temperature.

TEA as used herein refers to triethylamine.

TFA as used herein refers to trifluoroacetic acid.

THF as used herein refers to tetrahydrofuran.

t-Bu as used herein refers to the group tertiary butyl.

SPE as used herein refers to solid phase extraction, for example using columns containing silica gel for mini-chromatography.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals, and suitably refers to humans. Thus the methods of the present application are applicable to both human therapy and veterinary applications. In an embodiment, the subject is a mammal. In another embodiment, the subject is human.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject. One non-limiting example of such a carrier is a pharmaceutically acceptable oil typically used for parenteral administration.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

In general, prodrugs will be functional derivatives of the compounds of the application which are readily convertible in vivo into the compound from which it is notionally derived. Prodrugs of the compounds of the application may be conventional esters formed with the available hydroxyl and/or amino group. For examples, the available OH and/or NH₂ in the compounds of the application may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the application are those in which the hydroxyl and/or amino groups in the compounds is masked as groups which can be converted to hydroxyl and/or amino groups in vivo. Conventional procedures for the selection and preparation of suitable prodrugs are described, for example, in “Design of Prodrugs” ed. H. Bundgaard, Elsevier, 1985.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition described herein to prevent recurrence. Treatment methods comprise administering to a subject a therapeutically effective amount of one or more of the compounds of the application and optionally consist of a single administration, or alternatively comprise a series of administrations. For example, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the patient.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition treatable by inhibition of EGFR activity and particularly using an EGFR inhibitor, such as a compound of the application herein described.

The term “mediated by EGFR” as used herein means that the disease, disorder or condition to be treated is affected by, modulated by and/or has some biological basis, either direct or indirect, that includes aberrant EGFR activity, in particular, increased EGFR activity or, also, decreased EGFR activity such as results from mutation or splice variation and the like. These diseases respond favourably when EGFR activity associated with the disease is blocked by one or more of the present compounds.

As used herein, the term “effective amount” or “therapeutically effective amount” means an amount of one or more compounds of the application that is effective, at dosages and for periods of time necessary to achieve the desired result. For example in the context of treating a disease, disorder or condition, an effective amount is an amount that, for example, inhibits EGFR activity compared to the inhibition without administration of the one or more compounds. In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The term “administered” as used herein means administration of a therapeutically effective amount of one or more compounds or compositions of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer). Exemplary neoplastic disorders include but are not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

The term “cancer” as used herein refers to cellular-proliferative disease states.

II. Compounds and Compositions of the Application

Compounds of the present application were prepared and were found to inhibit uncontrolled and/or abnormal cellular activities affected directly or indirectly by EGFR protein. In particular, compounds of the present application exhibited activity as EGFR inhibitors, and are therefore useful in therapy, for example for the treatment of neoplastic disorders such as cancer.

Accordingly, one aspect of the present application includes a compound of Formula I or a pharmaceutically acceptable salt, solvate and/or prodrug thereof:

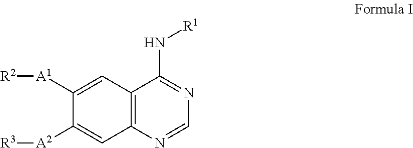

wherein:
R¹ is selected from unsubstituted or substituted aryl and unsubstituted or substituted heteroaryl, wherein the substituents for R¹ are selected from one or more of halogen, C₁₋₆alkyl, haloC₁₋₆alkyl, CN, C(O)R⁴, OR⁴, SR⁴, NR⁴R⁵, C(O)OR⁴, C(O)NR⁴R⁵, S(O)R⁴, SO₂R⁴, OC(O)R⁴, OC(O)OR⁴, OC(O)NR⁴R⁵, OC(S)NR⁴R⁵, OS(O)R⁴, OSO₂R⁴, NR⁴(OR⁵), NR⁶C(O)NR⁴R⁵, NR⁶C(S)NR⁴R⁵, NR⁵C(O)OR⁴, NR⁵C(S)OR⁴, NR⁵C(O)R⁴, C₁₋₆alkyleneC(O)R⁴, C₁₋₆alkyleneOR⁴, C₁₋₆alkyleneSR⁴, C₁₋₆alkyleneNR⁴R⁵, C₁₋₆alkyleneC(O)OR⁴, C₁₋₆alkyleneC(O)NR⁴R⁵, C₁₋₆alkyleneS(O)R⁴, C₁₋₆alkyleneSO₂R⁴, C₁₋₆alkyleneOC(O)R⁴, C₁₋₆alkyleneOC(O)OR⁴, C₁₋₆alkyleneOC(O)NR⁴R⁵, C₁₋₆alkyleneOC(S)NR⁴R⁵, C₁₋₆alkyleneOS(O)R⁴, C₁₋₆alkyleneOSO₂R⁴, C₁₋₆alkyleneNR⁴(OR⁵), C₁₋₆alkyleneNR⁶C(O)NR⁴R⁵, C₁₋₆alkyleneNR⁶C(S)NR⁴R⁵, C₁₋₆alkyleneNR⁵C(O)OR⁴, C₁₋₆alkyleneNR⁵C(S)OR⁴, C₁₋₆alkyleneNR⁵C(O)R⁴, C₂₋₆alkynyl, C₂₋₆alkynyleneC(O)R⁴, C₂₋₆alkynyleneOR⁴, C₂₋₆alkynyleneSR⁴, C₂₋₆alkynyleneNR⁴R⁵, C₂₋₆alkynyleneC(O)OR⁴, C₂₋₆alkynyleneC(O)NR⁴R⁵, C₂₋₆alkynyleneS(O)R⁴, C₂₋₆alkynyleneSO₂R⁴, C₂₋₆alkynyleneOC(O)R⁴, C₂₋₆alkynyleneOC(O)OR⁴, C₂₋₆alkynyleneOC(O)NR⁴R⁵, C₂₋₆alkynyleneOC(S)NR⁴R⁵, C₂₋₆alkynyleneOS(O)R⁴, C₂₋₆alkynyleneOSO₂R⁴, C₂₋₆alkynyleneNR⁴(OR⁵), C₂₋₆alkynyleneNR⁶C(O)NR⁴R⁵, C₂₋₆alkynyleneNR⁶C(S)NR⁴R⁵, C₂₋₆alkynyleneNR⁵C(O)OR⁴, C₂₋₆alkynyleneNR⁵C(S)OR⁴, C₂₋₆alkynyleneNR⁵C(O)R⁴ and 3-7 membered heterocycloalkyl;
R² and R³ are independently selected from C₁₋₂₀alkyl, C₆₋₂₀aryl, heteroaryl, C₃₋₂₀cycloalkyl, heterocycloalkyl, C₁₋₁₀alkyleneC₆₋₂₀aryl, C₁₋₁₀alkyleneheteroaryl, C₁₋₁₀alkyleneC₃₋₂₀cycloalkyl, C₁₋₁₀alkyleneheterocycloalkyl, C(O)C₁₋₂₀alkyl, C(O)C₆₋₂₀aryl, C(O)heteroaryl, C(O)C₃₋₂₀cycloalkyl, C(O)NR⁶heterocycloalkyl, C(O)NR⁶C₁₋₂₀alkyl, C(O)NR⁶C₆₋₂₀aryl, C(O)NR⁶heteroaryl, C(O)NR⁶C₃₋₂₀cycloalkyl and C(O)NR⁶heterocycloalkyl, wherein R² and R³ are unsubstituted or substituted with one or more substituents independently selected from halo, C₁₋₆alkyl, OC₁₋₆alkyl, halo-substituted C₁₋₆alkyl, halo-substituted OC₁₋₆alkyl, halo-substituted SC₁₋₆alkyl halo-substituted C₁₋₆alkyleneOC₁₋₆alkyl, halo-substituted C₁₋₆alkyleneSC₁₋₆alkyl, halo-substituted C₁₋₆alkyleneS(O)C₁₋₆alkyl, halo-substituted C₁₋₆alkyleneSO₂C₁₋₆alkyl and C₁₋₆alkyleneOhalo-substituted C₁₋₆alkyl, provided that at least one of R² and R³ comprises at least one fluorine atom;
R⁴, R⁵ and R⁶ are independently selected from H, C₆₋₁₀aryl, heteroaryl, C₃₋₁₀cycloalkyl, C₃₋₁₀heterocycloalkyl, haloC₁₋₆alkyl and C₁₋₆alkyl; and
A¹ and A² are independently selected from CH₂, O, S, S(O), SO₂NH and NR⁵.

In an embodiment, R¹ is selected from unsubstituted or substituted aryl and unsubstituted or substituted heteroaryl, wherein the substituents for R¹ are selected from one to four of halogen, C₁₋₆alkyl, haloC₁₋₆alkyl, CN, C(O)R⁴, OR⁴, NR⁴R⁵, C(O)OR⁴, C(O)NR⁴R⁵, C₁₋₆alkyleneC(O)R⁴, C₁₋₆alkyleneOR⁴, C₁₋₆alkyleneNR⁴R⁵, C₁₋₆alkyleneC(O)OR⁴, C₁₋₆alkyleneC(O)NR⁴R⁵, C₂₋₆alkynyl, C₂₋₆alkynyleneC(O)R⁴, C₂₋₆alkynyleneOR⁴, C₂₋₆alkynyleneNR⁴R⁵, C₂₋₆alkynyleneC(O)OR⁴, C₂₋₆alkynyleneC(O)NR⁴R⁵ and 5-6 membered heterocycloalkyl, in which R⁴ and R⁵ are independently selected from haloC₁₋₆alkyl and C₁₋₆alkyl.

In an embodiment, R¹ is selected from unsubstituted or substituted aryl wherein the substituents for R¹ are selected from one to four of halogen, C₁₋₆alkyl, haloC₁₋₆alkyl, CN, C(O)R⁴, OR⁴, NR⁴R⁵, C(O)OR⁴, C(O)NR⁴R⁵, C₁₋₆alkyleneC(O)R⁴, C₁₋₆alkyleneOR⁴, C₁₋₆alkyleneNR⁴R⁵, C₁₋₆alkyleneC(O)OR⁴, C₁₋₆alkyleneC(O)NR⁴R⁵, C₂₋₆alkynyl, C₂₋₆alkynyleneC(O)R⁴, C₂₋₆alkynyleneOR⁴, C₂₋₆alkynyleneNR⁴R⁵, C₂₋₆alkynyleneC(O)OR⁴, C₂₋₆alkynyleneC(O)NR⁴R⁵ and 5-6 membered heterocycloalkyl, in which R⁴ and R⁵ are independently selected from haloC₁₋₆alkyl and C₁₋₆alkyl.

In an embodiment, R¹ is selected from substituted aryl wherein the substituents of R¹ are selected from one to four of Cl, F, CF₃, OR⁴, NR⁴R⁵ and C₂₋₆alkynyl in which R⁴ and R⁵ are independently selected from fluoroC₁₋₆alkyl and C₁₋₆alkyl. In another embodiment, R¹ is selected from substituted aryl wherein the substituents of R¹ are selected from one to three of Cl, F, CF₃, OR⁴, NR⁴R⁵ and C₂₋₆alkynyl in which R⁴ and R⁵ are independently selected from CF₃, CHF₂ and CH₃. In another embodiment, R¹ is selected from substituted aryl wherein the substituents of R¹ are selected from one to three of Cl, F and C₂₋₆alkynyl. In a further embodiment, R¹ is selected from substituted heteroaryl wherein the substituents of R¹ are selected from one to three of Cl, F, CF₃, OR⁴, NR⁴R⁵ and C₂₋₆alkynyl and R⁴ and R⁵ are independently selected from fluoroC₁₋₆alkyl and C₁₋₆alkyl.

In an embodiment, R² and R³ are independently selected from C₁₋₁₀alkyl, C₆₋₁₀aryl, C₅₋₁₀heteroaryl, C₃₋₁₀cycloalkyl, C₅₋₁₀heterocycloalkyl, C₁₋₆alkyleneC₆₋₁₉aryl, C₁₋₆alkyleneC₅₋₁₀heteroaryl, C₁₋₆alkyleneC₅₋₁₀cycloalkyl, C₁₋₆alkyleneC₅₋₁₀heterocycloalkyl, C(O)C₁₋₁₀alkyl, C(O)C₆₋₁₀aryl, C(O)C₅₋₁₀heteroaryl, C(O)C₃₋₁₀cycloalkyl, C(O)NR⁶heterocycloalkyl, C(O)NR⁶C₁₋₁₀alkyl, C(O)NR⁶C₆₋₁₀aryl, C(O)NR⁶C₅₋₁₀heteroaryl, C(O)NR⁶C₃₋₁₀cycloalkyl and C(O)NR⁶C₅₋₁₀heterocycloalkyl, wherein R² and R³ are unsubstituted or substituted with one or four substituents independently selected from halo, C₁₋₆alkyl, OC₁₋₆alkyl, fluoro-substituted C₁₋₆alkyl, fluoro-substituted OC₁₋₆alkyl, fluoro-substituted SC₁₋₆alkyl fluoro-substituted C₁₋₆alkyleneOC₁₋₆alkyl, fluoro-substituted C₁₋₆alkyleneSC₁₋₆alkyl, fluoro-substituted C₁₋₆alkyleneS(O)C₁₋₆alkyl, fluoro-substituted C₁₋₆alkyleneSO₂C₁₋₆alkyl and C₁₋₆alkyleneOfluoro-substituted C₁₋₆alkyl, provided that at least one of R² and R³ comprises at least one fluorine atom.

In an embodiment, R² and R³ are independently selected from C₁₋₁₀alkyl, C₁₋₆alkyleneC₆₋₁₉aryl, C₁₋₆alkyleneC₅₋₁₀heteroaryl, C₁₋₆alkyleneC₅₋₁₀cycloalkyl, C₁₋₆alkyleneC₅₋₁₀heterocycloalkyl, C(O)C₁₋₁₀alkyl, C(O)C₆₋₁₀aryl, C(O)C₅₋₁₀heteroaryl, C(O)C₃₋₁₀cycloalkyl, C(O)NR⁶heterocycloalkyl, C(O)NR⁶C₁₋₁₀alkyl, C(O)NR⁶C₆₋₁₀aryl, C(O)NR⁶C₅₋₁₀heteroaryl, C(O)NR⁶C₃₋₁₀cycloalkyl and C(O)NR⁶C₅₋₁₀heterocycloalkyl, wherein R² and R³ are unsubstituted or substituted with one or more substituents independently selected from halo, C₁₋₆alkyl, OC₁₋₆alkyl, fluoro-substituted C₁₋₆alkyl, fluoro-substituted OC₁₋₆alkyl, fluoro-substituted SC₁₋₆alkyl fluoro-substituted C₁₋₆alkyleneOC₁₋₆alkyl, fluoro-substituted C₁₋₆alkyleneSC₁₋₆alkyl, fluoro-substituted C₁₋₆alkyleneS(O)C₁₋₆alkyl, fluoro-substituted C₁₋₆alkyleneSO₂C₁₋₆alkyl and C₁₋₆alkyleneOfluoro-substituted C₁₋₆alkyl, provided that at least one of R² and R³ comprises at least one fluorine atom.

In an embodiment, R² and R³ are independently selected from:

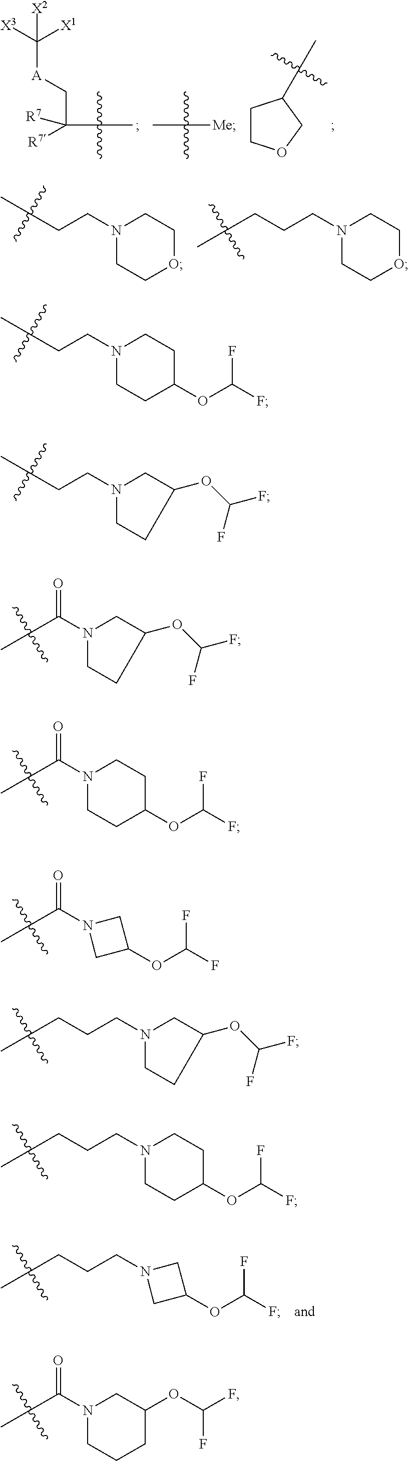

wherein R⁷ and R^{7′} are independently selected from H, aryl, heteroaryl and C₁₋₆alkyl; A is CH₂, O, S, NH or NC₁₋₆alkyl; and X¹, X² and X₃ are the same or different and are selected from H, halo and C₁₋₆alkyl. In an embodiment, R⁷ and R^{7′} are independently selected from H and C₁₋₄alkyl; A is CH₂ or O; and X¹, X² and X₃ are the same or different and are selected from H, F and C₁₋₄alkyl. In an embodiment, R⁷ and R^{7′} are independently selected from H and CH³; A is CH₂ or O; and X¹, X² and X₃ are the same or different and are selected from H and F.

In an embodiment, R² and R³ are independently selected from:

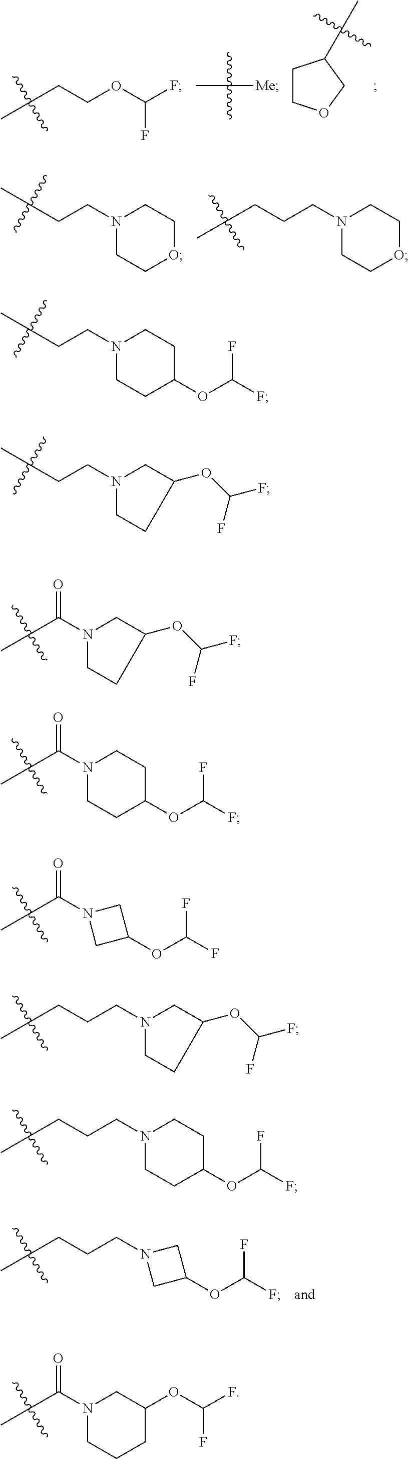

In an embodiment, both of R² and R³ are

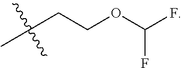 [SMILES: *CCOC(F)F]

In another embodiment, one of is R² and R³ is

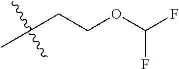 [SMILES: *CCOC(F)F]

and the other of R² and R³ is CH₃.

In an embodiment R⁴, R⁵ and R⁶ are independently selected from H, haloC₁₋₆alkyl and C₁₋₆alkyl. In an embodiment R⁴, R⁵ and R⁶ are independently selected from H, CF₃, CHF₂ and CH₃.

In an embodiment A¹ and A² are independently selected from CH₂, O, NH and NCH₃. In an embodiment, both of A¹ and A² are O. In an embodiment one of A¹ and A² is O and the other of A¹ and A² is NH.

In an embodiment, the compound of the application is selected from:

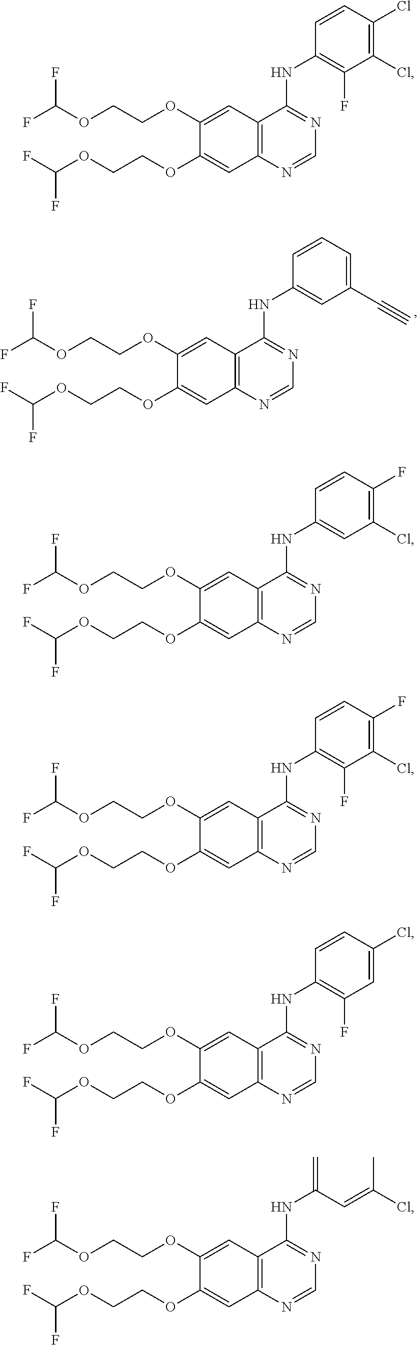 [SMILES: Fc1c(Nc2ncnc3cc(OCCOC(F)F)c(OCCOC(F)F)cc23)ccc(Cl)c1Cl ; C#Cc1cccc(Nc2ncnc3cc(OCCOC(F)F)c(OCCOC(F)F)cc23)c1 ; Fc1ccc(Nc2ncnc3cc(OCCOC(F)F)c(OCCOC(F)F)cc23)cc1Cl ; Fc1ccc(Nc2ncnc3cc(OCCOC(F)F)c(OCCOC(F)F)cc23)c(F)c1Cl ; Fc1cc(Cl)ccc1Nc1ncnc2cc(OCCOC(F)F)c(OCCOC(F)F)cc12 ; C=C(/C=C(\C)Cl)Nc1ncnc2cc(OCCOC(F)F)c(OCCOC(F)F)cc12]

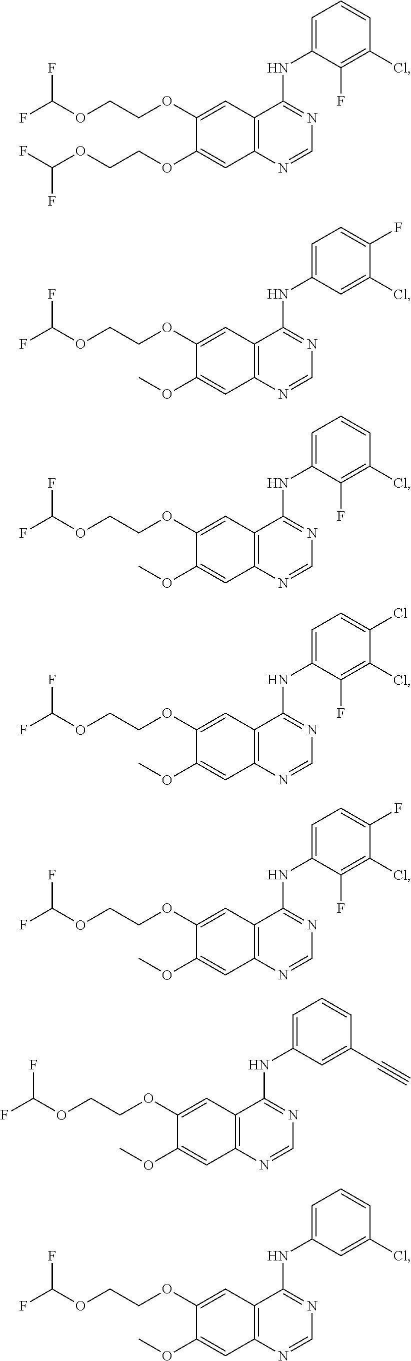

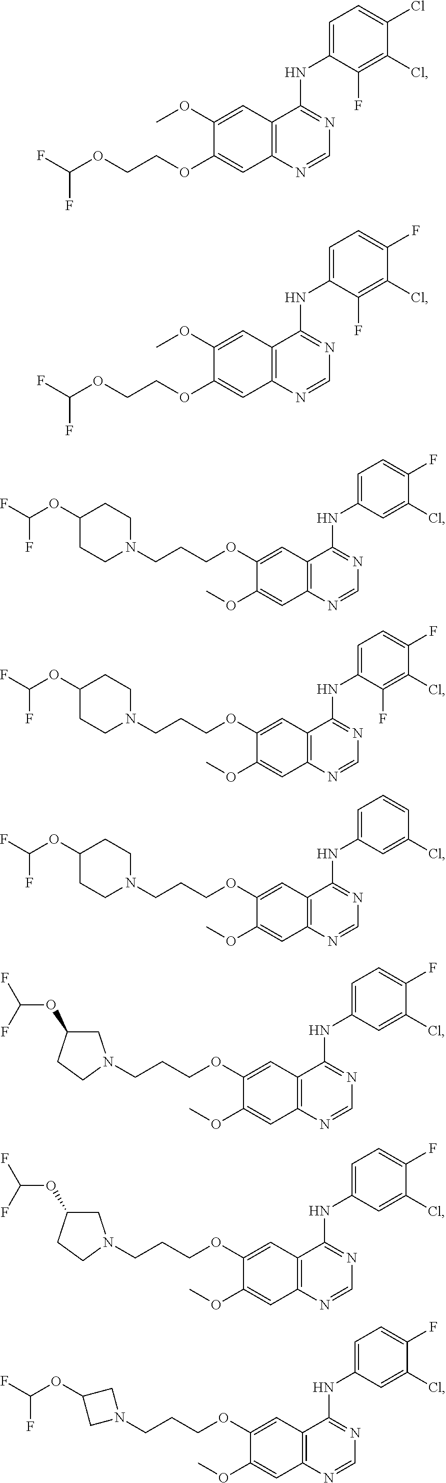

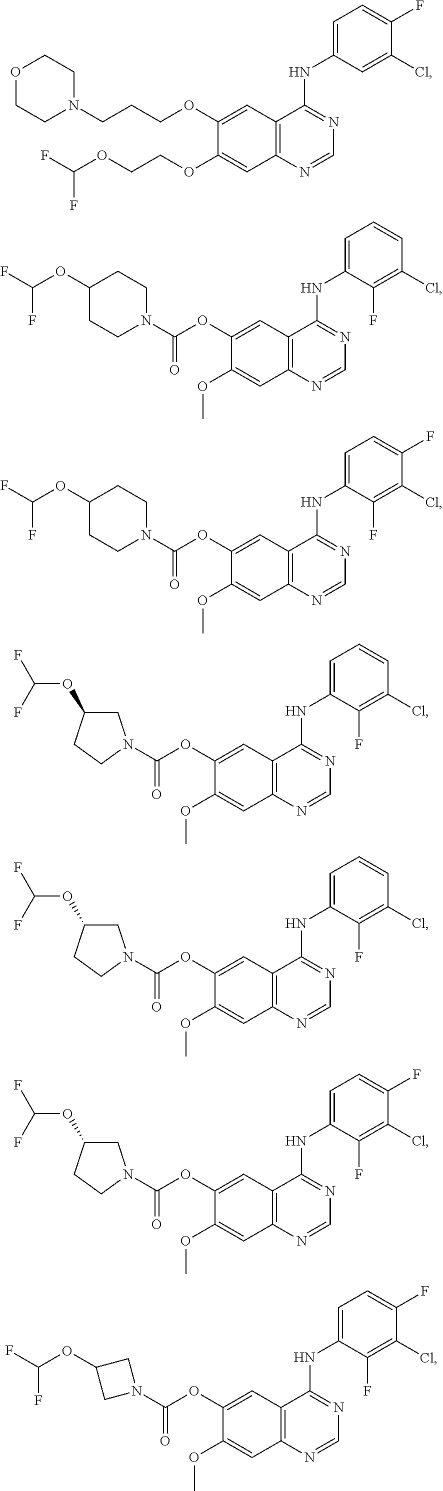

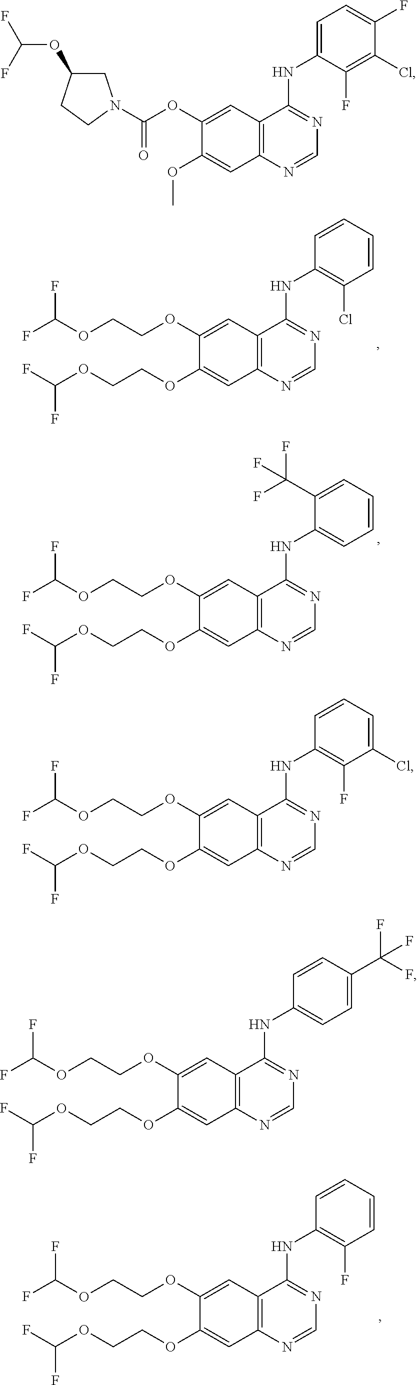 [SMILES: COc1cc2ncnc(Nc3ccc(F)c(Cl)c3F)c2cc1OC(=O)N1CC[C@@H](OC(F)F)C1 ; FC(F)OCCOc1cc2ncnc(Nc3ccccc3Cl)c2cc1OCCOC(F)F ; FC(F)OCCOc1cc2ncnc(Nc3ccccc3C(F)(F)F)c2cc1OCCOC(F)F ; Fc1c(Cl)cccc1Nc1ncnc2cc(OCCOC(F)F)c(OCCOC(F)F)cc12 ; FC(F)OCCOc1cc2ncnc(Nc3ccc(C(F)(F)F)cc3)c2cc1OCCOC(F)F ; Fc1ccccc1Nc1ncnc2cc(OCCOC(F)F)c(OCCOC(F)F)cc12]

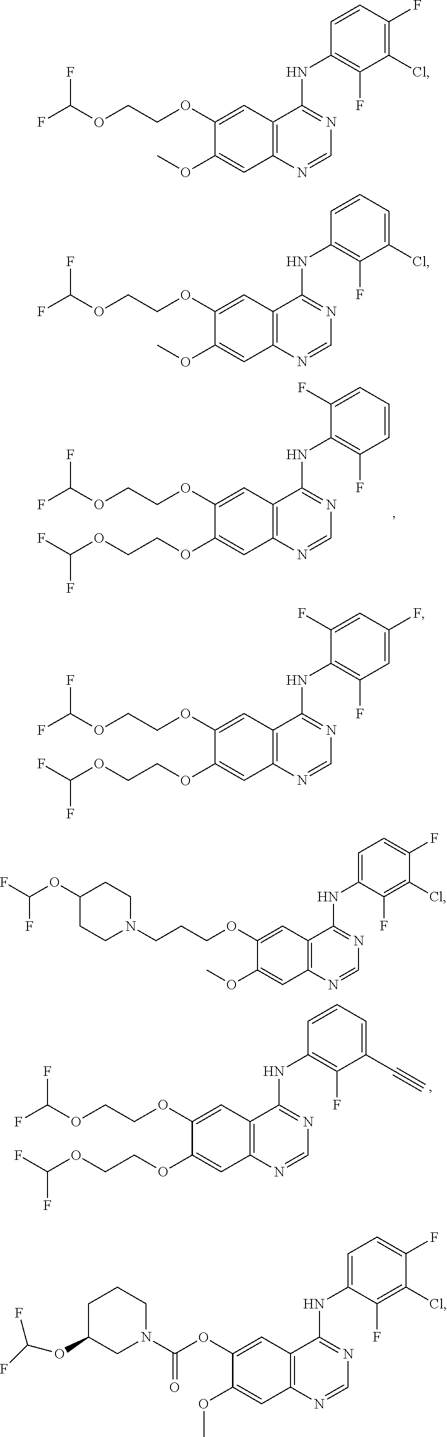

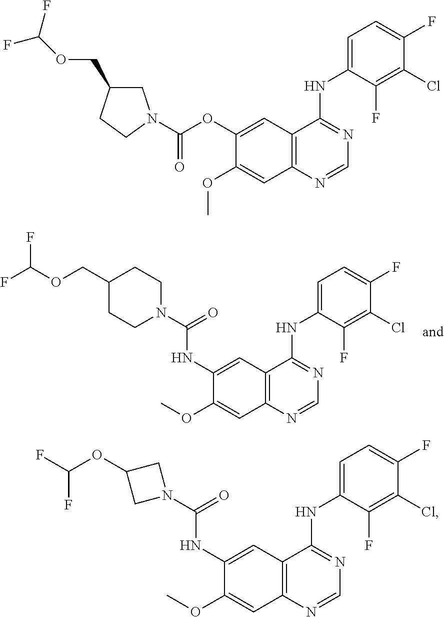 [SMILES: COc1cc2ncnc(Nc3ccc(F)c(Cl)c3F)c2cc1OC(=O)N1CC[C@@H](COC(F)F)C1 ; COc1cc2ncnc(Nc3ccc(F)c(Cl)c3F)c2cc1NC(=O)N1CCC(COC(F)F)CC1 ; COc1cc2ncnc(Nc3ccc(F)c(Cl)c3F)c2cc1NC(=O)N1CC(OC(F)F)C1]

or a pharmaceutically acceptable salt, solvate or prodrug thereof.

As noted above, all stereoisomers are included within the scope of the present application. Therefore, while a specific stereochemistry is shown in the above compounds, the present application includes compounds having the alternate stereochemistry as well as mixtures thereof in any proportion.

In an embodiment of the application there is also included a compound of Formula I or a pharmaceutically acceptable salt, solvate or prodrug thereof:

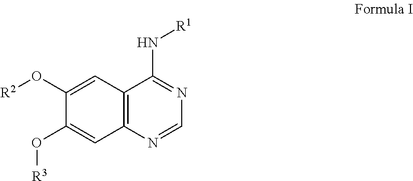

wherein:
R¹ is aryl or heteroaryl (which optionally has one or more substituents selected from halo, CN, CF₃, OR⁴, SR⁴, N(R⁴)₂, and 3-7 membered heterocycloalkyl);
R² and R³ are independently selected from

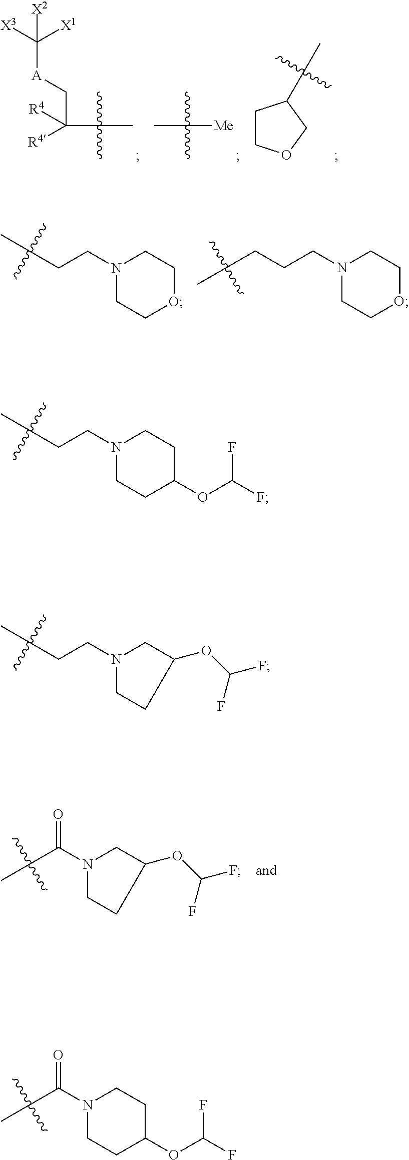

R⁴ and R^{4′} is independently selected from H, aryl, heteroaryl and C₁₋₆ alkyl; such that
A is CH₂, O, S or NR⁴; and
X¹, X², and X³ are the same or different and are selected from H, halo and lower alkyl.

In an embodiment, compounds of Formula I, wherein R² is selected from:

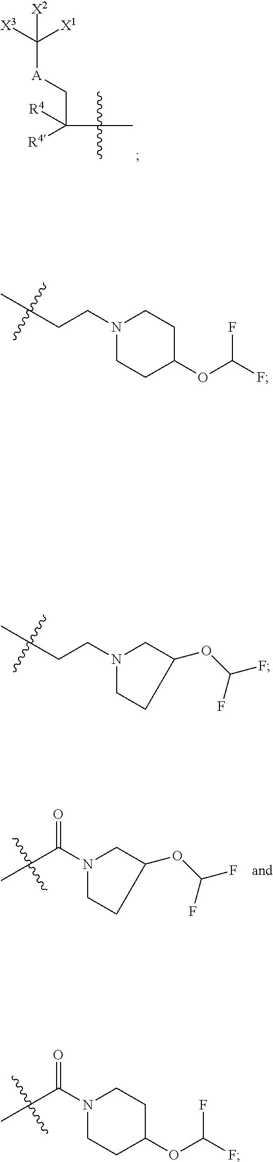

and R³ is selected from:

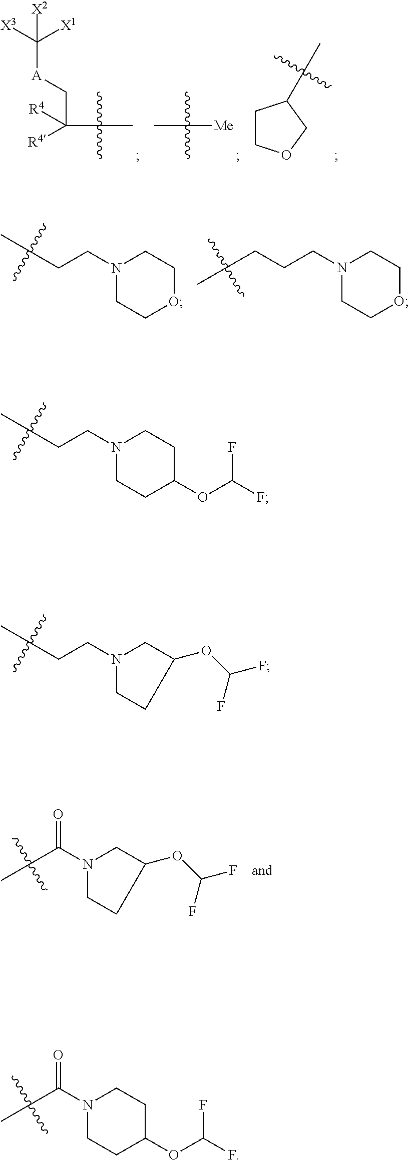

In another embodiment, compounds of Formula I, wherein R₃ is selected from:

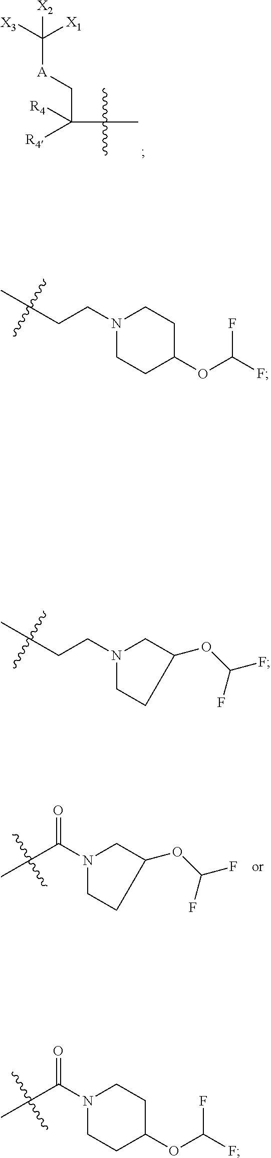

### and R₂ is

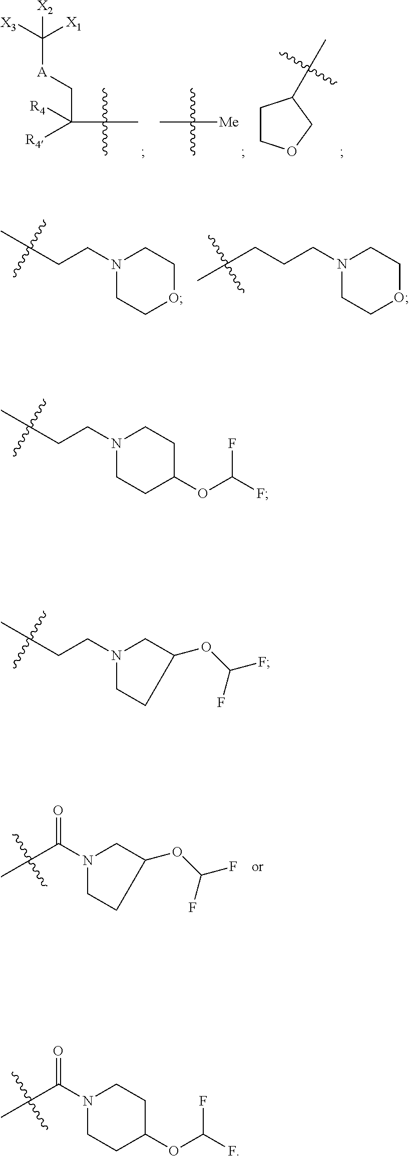

In a further embodiment, compounds of Formula I, wherein R¹ represents aryl optionally substituted with halogen.

In another embodiment, the compounds of Formula I are:

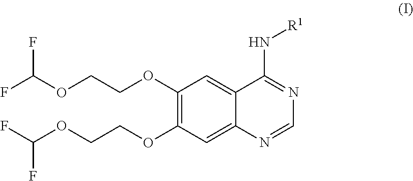

wherein R¹ is a phenyl or napthyl group substituted with 1, 2 or 3 substituents independently selected from Cl, F, CF₃, CH₃ and C≡CH.

In an embodiment, the compound of the present application is selected from the compounds of Formula I in Table 1 or a pharmaceutically acceptable salt, solvate or prodrug thereof.

### Preparation of Compounds

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art.

The compounds of Formula I generally can be prepared according to the process illustrated in Scheme I. Variables in the following schemes are as defined above for Formula I unless otherwise specified.

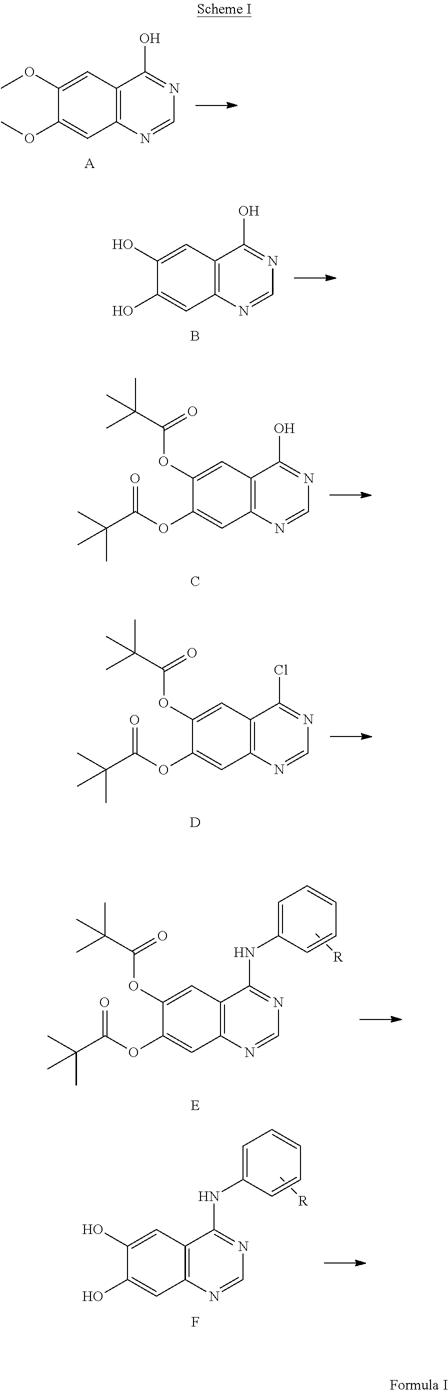

As shown in Scheme 1, the compounds of the present application can be prepared by acid mediated ether cleavage of the commercial quinazoline A to give intermediate B. Subsequent acylation of intermediate B with pivaloyl chloride give the diester C. Chlorination of C with POCl₃ affords the chloro-qunazoline D. Nucleophilic displacement of Chloro- with anilines affords intermediated E. Hydrolysis of E with ammonia to give diphenol F follow by simultaneous or sequential alkylation, acylation or carbamoylation afford compounds of Formula I.

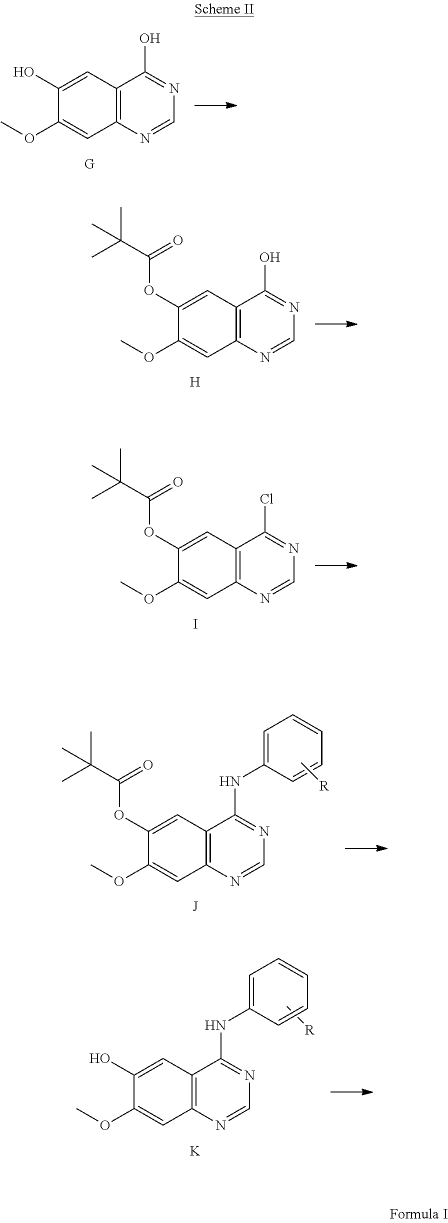

As shown in Scheme II, the compounds of the present application can be prepared by acylation of commercial G with pivaloyl chloride to give the ester H. Chlorination of H with POCl3 affords the chloro-qunazoline I. Nucleophilic displacement of Chloro- with anilines affords intermediated J. Hydrolysis of J with ammonia to give phenol K follow by alkylation, acylation or carbamoylation afford compounds of Formula I.

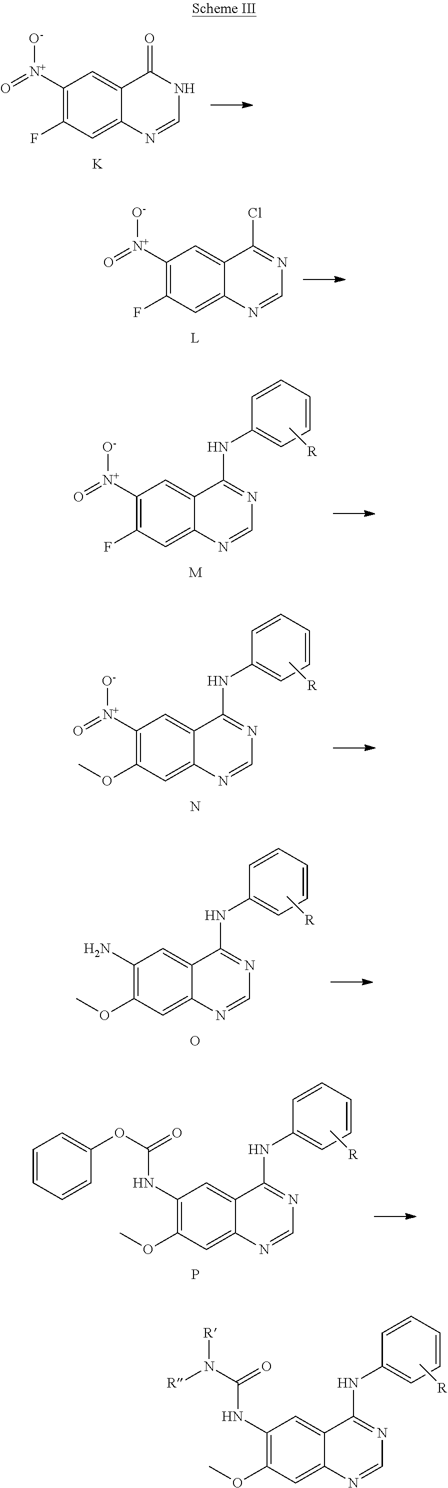

As shown in Scheme III, the compounds of the present application can be prepared by chlorination of commercial fluoro-nitro-K followed by chloride displacement with a suitable aniline to give M. The fluoride N is displaced with methoxide to give N which is reduced with Raney Nickel to give aniline O. Urea formation gives compounds of formula II.

Amines are obtained from commercial sources or prepared by methods known in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of nay of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multidose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the application including pharmaceutically acceptable salts, solvates and/or prodrugs thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

A compound of the application is either used alone or in combination with other known agents useful for treating diseases, disorders or conditions that treatable by inhibition of EGFR, and those that are treatable with a EGFR inhibitor. When used in combination with other agents useful in treating diseases, disorders or conditions treatable by inhibition of EGFR, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the one or more compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. In embodiments of the application the one or more compounds of the application are administered in a single daily, weekly or monthly dose or the total daily dose is divided into two, three or four daily doses.

In the above, the term “a compound” also includes embodiments wherein one or more compounds are referenced.

III. Methods and Uses of the Application

The compounds of the application have been shown to be capable of inhibiting EGFR activity.

Accordingly, the present application includes a method for inhibiting EGFR in a cell, either in a biological sample or in a patient, comprising administering an effective amount of one or more compounds of the application to the cell. The application also includes a use of one or more compounds of the application for inhibiting EGFR in a cell as well as a use of one or more compounds of the application for the preparation of a medicament for inhibiting EGFR in a cell. The application further includes one or more compounds of the application for use in inhibiting EGFR in a cell.

As the compounds of the application have been shown to be capable of inhibiting EGFR protein activity, the compounds of the application are useful for treating diseases, disorders or conditions by the inhibition of EGFR. Therefore the compounds of the present application are useful as medicaments. Accordingly, the present application includes a compound of the application for use as a medicament.

The present application also includes a method of treating a disease, disorder or condition by inhibition of EGFR comprising administering a therapeutically effective amount of one or more compounds of the application to a subject in need thereof.

The present application also includes a use of one or more compounds of the application for treatment of a disease, disorder or condition by inhibition of EGFR as well as a use of one or more compounds of the application for the preparation of a medicament for treatment of a disease, disorder or condition by inhibition of EGFR. The application further includes one or more compounds of the application for use in treating a disease, disorder or condition by inhibition of EGFR.

In an embodiment, the disease, disorder or condition is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to be effective against the cell lines of a 60 human tumor cell line panel. Therefore in another embodiment of the present application, the disease, disorder or condition requiring inhibition of EGFR is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is a solid cancer or a so-called liquid cancer, and can be selected from a cancer of the skin, blood, prostate, colorectum, pancreas, kidney, ovary, breast, for example mammary, liver, tongue and lung. In another embodiment, the cancer is selected from leukaemia, lymphoma, non-Hodgkin's lymphoma and multiple myeloma. The cancer target includes particularly those for which regulatory approval has already been granted for other EGFR inhibitors. These cancers include colorectal cancer, head and neck cancer, pancreatic cancer, non-small cell lung cancer, and glioma.

In an embodiment, the disease, disorder or condition is a disease, disorder or condition associated with an uncontrolled and/or abnormal cellular activity affected directly or indirectly by alteration of EGFR protein activity. In another embodiment, the uncontrolled and/or abnormal cellular activity that is affected directly or indirectly by altered EGFR activity is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell.

The present application also includes a method of inhibiting uncontrolled and/or abnormal cellular activities affected directly or indirectly by EGFR protein in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by EGFR protein in a cell as well as a use of one or more compounds of the application for the preparation of a medicament for inhibition of uncontrolled and/or abnormal cellular activities affected directly or indirectly by EGFR protein inhibition in a cell. The application further includes one or more compounds of the application for use in inhibiting uncontrolled and/or abnormal cellular activities affected directly or indirectly by EGFR. Accordingly, the present application also includes a method of treating a disease, disorder or condition that is treatable by inhibition of EGFR comprising administering a therapeutically effective amount of one or more compounds of the application in combination with another known agent useful for such treatment. The present application also includes a use of one or more compounds of the application in combination with another known agent useful for treatment of a disease, disorder or condition mediated by inhibition of EGFR for treatment of a disease, disorder or condition mediated by inhibition of EGFR as well as a use of one or more compounds of the application in combination with another known agent useful for treatment of a disease, disorder or condition mediated by EGFR, for the preparation of a medicament for treatment of a disease, disorder or condition treatable by inhibition of EGFR. The application further includes one or more compounds of the application in combination with another known agent useful for treatment of a disease, disorder or condition treatable by inhibition of EGFR for use in treating a disease, disorder or condition mediated by EGFR. In an embodiment, the disease, disorder or condition treatable by inhibition of EGFR is a cancer such as multiple myeloma, lymphoma, leukemia, ovarian cancer, brain cancer, lung cancer, and pancreatic cancer. Treatable EGFR-mediated cancers thus include benign or malignant tumors (e.g., renal, liver, kidney, bladder, breast, gastric, ovarian, colorectal, prostate, pancreatic, lung, vulva, and thyroid); hepatic carcinomas; sarcomas; glioblastomas; and various head and neck tumors including particularly head and neck cancers and especially squamous cell carcinoma of the head and neck, colorectal cancers, gastrointestinal cancers, brain tumours including glioblastomas, and tumours of the lung including non-small-cell lung carcinoma, and of the breast, pancreas, esophagus, kidney, ovary, cervix and prostate.

In a further embodiment, the disease, disorder or condition mediated by EGFR is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

### EXAMPLES

The following non-limiting examples are illustrative of the present application:

The introduction of the fluorine atom into molecules may bring about changes in the physical and/or chemical properties of the parent molecules, for example it may result in the enhancement of pharmacokinetic properties and/or biological activities. Replacement of hydrogen atoms may also result in improved thermal and metabolic stability. Improved metabolic stability is generally a desirable feature since the possibility exist that in vivo decomposition may produce toxic effects. The properties of the fluorine atom include its small size, low polarizability, high electronegativity and its ability to form strong bonds with carbon. Accordingly, bioactive compounds containing fluorinated groups such as —OCHF₂ are useful.

The geminal combination of an alkoxy or aryloxy group with a fluorine atom offers the possibility of bonding/nonbonding resonance, which can be formally expressed by the superposition of a covalent and ionic limiting structure. This phenomenon, which reveals itself as a lengthening and weakening of the carbon-halogen bond and a shortening and strengthening of the carbon-oxygen bond is known as the generalized anomeric effect [Schlosser et al. Chem. Rev. 2005, 105, 827-856].

### Example 1

### A. General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Example 2: Representative Synthesis of Compounds of Formula I

### Synthesis of Chloroquinazoline Intermediate:

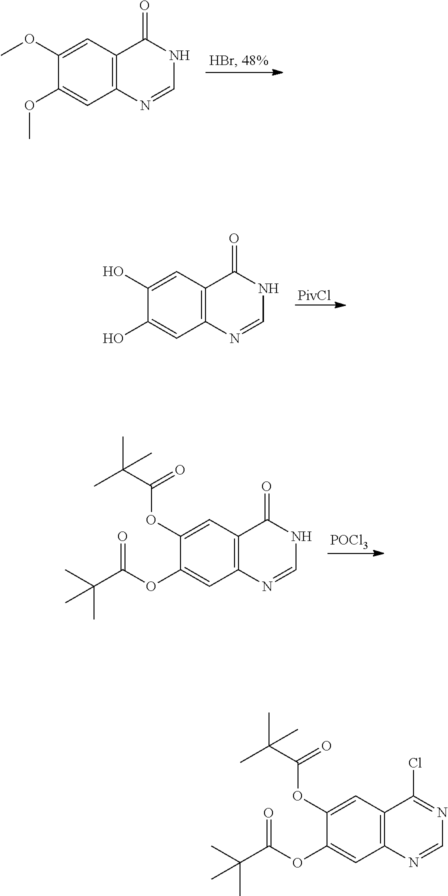

### (i) 6,7-dihydroxy-3H-quinazolin-4-one

6,7-dimethoxy-3H-quinazolin-4-one (25 g, 124 mmol) was stirred in HBr, 48% (150 mL) at 120° C. overnight. The mixture was cooled to room temperature and filtered. The filter cake was stirred in water and treated with ammonium hydroxide to pH=8 and the mixture was filtered. The filter cake was stirred in acetone and the resulting mixture was filtered. The filter cake was washed with diethyl ether and dried giving the desired product as a fine, pale powder (21 g, 97%). ¹H NMR (d6-DMSO) δ 11.82 (brs, 1H), 10.13 (s, 1H), 9.75 (s, 1H), 7.84 (s, 1H), 7.34 (s, 1H), 6.92 (s, 1H).

### (ii) [7-(2,2-dimethylpropanoyloxy)-4-oxo-3H-quinazolin-6-yl] 2,2-dimethylpropanoate

To a stirred suspension of 6,7-dihydroxy-3H-quinazolin-4-one (10 g, 56 mmol) in DMF (50 mL) was added triethylamine (17.0 g, 168 mmol) followed by pivaloyl chloride (20.3 g, 168 mmol) slowly, over a period of 30 min. The mixture was stirred for a further 30 min at room temperature then diluted with ethyl acetate. The mixture was washed with water (1×), NaHCO3 (1×), water (2×) and brine (1×). The organic phase was dried, filtered and concentrated in vacuo then stirred in hexanes. The resulting suspension was filtered to collect the desired product as a fine white powder (10 g, 51%).

### (iii) [4-chloro-7-(2,2-dimethylpropanoyloxy)quinazolin-6-yl] 2,2-dimethylpropanoate

[7-(2,2-dimethylpropanoyloxy)-4-oxo-3H-quinazolin-6-yl] 2,2-dimethylpropanoate (6.3 g, 18.1 mmol) was stirred with DCE (60 mL) and triethylamine (10 mL, 72.4 mmol) then treated with POCl₃ (5.1 mL, 54.6 mmol). The resulting mixture was stirred at 70-80° C. for 3 h then cooled in an ice-water bath and quenched via addition of ice and water. The organic layer was separated and the aqueous phase was extracted with DCM (3×). The combined organics were washed with brine (1×), dried filtered and concentrated in vacuo giving the crude product used directly in the subsequent reaction (6.6 g quantitative).

### Incorporation of Aniline:

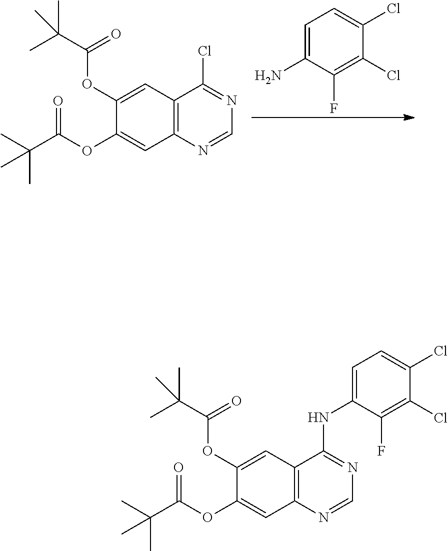 [SMILES: CC(C)(C)C(=O)Oc1cc2ncnc(Cl)c2cc1OC(=O)C(C)(C)C ; Nc1ccc(Cl)c(Cl)c1F ; CC(C)(C)C(=O)Oc1cc2ncnc(Nc3ccc(Cl)c(Cl)c3F)c2cc1OC(=O)C(C)(C)C]

### [4-(3,4-dichloro-2-fluoro-anilino)-7-(2,2-dimethylpropanoyloxy)quinazolin-6-yl] 2,2-dimethylpropanoate

To a stirred solution of [4-chloro-7-(2,2-dimethylpropanoyloxy)quinazolin-6-yl] 2,2-dimethylpropanoate (6.6 g, 18.1 mmol) in DCE (60 mL) was added HCl, 4 M in dioxane (9 mL) followed by 3,4-dichloro-2-fluoro-aniline (3.1 g, 17.2 mmol) and the resulting mixture was stirred at 70° C. for 1.5 h. The mixture was then cooled to room temperature and diluted with diethyl ether. The resulting suspension was filtered to collect the desired product (8.5 g, 92%).

The following compounds were made in a similar manner:

| Structure | Nomenclature | Appearance/Yield |
|---|---|---|
| 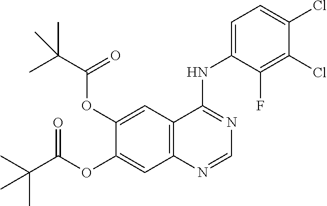 [SMILES: CC(C)(C)C(=O)Oc1cc2ncnc(Nc3ccc(Cl)c(Cl)c3F)c2cc1OC(=O)C(C)(C)C] | [4-(3,4-dichloro-2-fluoro- anilino)-7-(2,2- dimethylpropanoyloxy)quinazolin- 6-yl] 2,2-dimethylpropanoate | White solid, 92% |
| 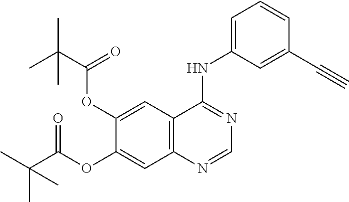 [SMILES: C#Cc1cccc(Nc2ncnc3cc(OC(=O)C(C)(C)C)c(OC(=O)C(C)(C)C)cc23)c1] | [7-(2,2-dimethylpropanoyloxy)- 4-(3-ethynylanilino)quinazolin- 6-yl] 2,2-dimethylpropanoate | White solid, 95% |
| 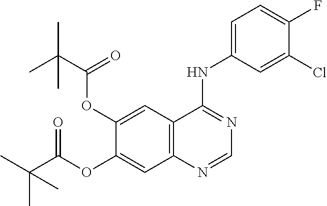 [SMILES: CC(C)(C)C(=O)Oc1cc2ncnc(Nc3ccc(F)c(Cl)c3)c2cc1OC(=O)C(C)(C)C] | [4-(3-chloro-4-fluoro-anilino)-7- (2,2- dimethylpropanoyloxy)quinazolin- 6-yl] 2,2-dimethylpropanoate | White solid, 95% |
| 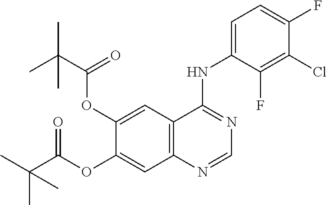 [SMILES: CC(C)(C)C(=O)Oc1cc2ncnc(Nc3ccc(F)c(Cl)c3F)c2cc1OC(=O)C(C)(C)C] | [4-(3-chloro-2,4-difluoro- anilino)-7-(2,2- dimethylpropanoyloxy)quinazolin- 6-yl] 2,2-dimethylpropanoate | Pale solid, 100% |
| 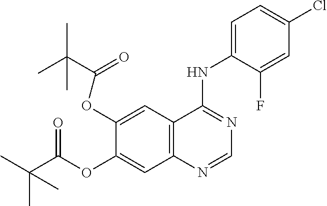 [SMILES: CC(C)(C)C(=O)Oc1cc2ncnc(Nc3ccc(Cl)cc3F)c2cc1OC(=O)C(C)(C)C] | [4-(4-chloro-2-fluoro-anilino)-7- (2,2- dimethylpropanoyloxy)quinazolin- 6-yl] 2,2-dimethylpropanoate | Off-white solid, 100% |
| 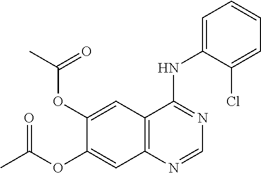 [SMILES: CC(=O)Oc1cc2ncnc(Nc3ccccc3Cl)c2cc1OC(C)=O] | [7-acetoxy-4-(2- chloroanilino)quinazolin-6-yl] acetate | Beige solid, 100% |
| 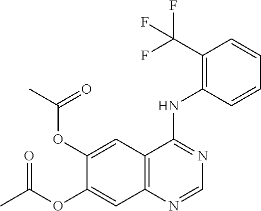 [SMILES: CC(=O)Oc1cc2ncnc(Nc3ccccc3C(F)(F)F)c2cc1OC(C)=O] | [7-acetoxy-4-[2- (trifluoromethyl)anilino]quinazolin- 6-yl] acetate | White solid, 29% |
| 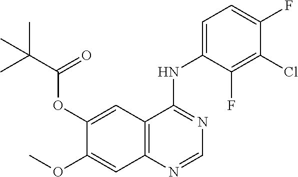 [SMILES: COc1cc2ncnc(Nc3ccc(F)c(Cl)c3F)c2cc1OC(=O)C(C)(C)C] | [4-(3-chloro-2,4-difluoro- anilino)-7-methoxy-quinazolin- 6-yl] 2,2-dimethylpropanoate | White solid, 93% |
| 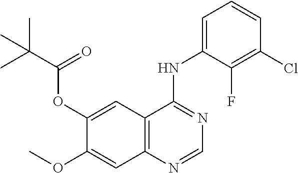 [SMILES: COc1cc2ncnc(Nc3cccc(Cl)c3F)c2cc1OC(=O)C(C)(C)C] | [4-(3-chloro-2-fluoro-anilino)-7- methoxy-quinazolin-6-yl] 2,2- dimethylpropanoate | White solid, 100% |
| 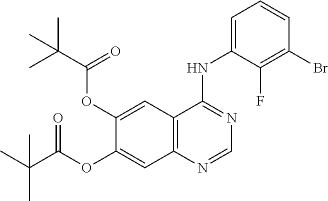 [SMILES: CC(C)(C)C(=O)Oc1cc2ncnc(Nc3cccc(Br)c3F)c2cc1OC(=O)C(C)(C)C] | [4-(3-bromo-2-fluoro-anilino)-7- (2,2- dimethylpropanoyloxy)quinazolin- 6-yl] 2,2-dimethylpropanoate | White solid, 100% |

[7-(2,2-dimethylpropanoyloxy)-4-(3-ethynylanilino)quinazolin-6-yl] 2,2-dimethylpropanoate: The desired product was obtained as a white solid (1.0 g, 95%).

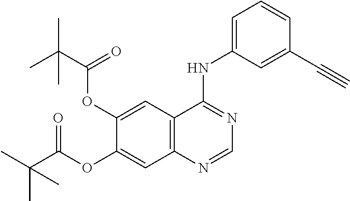 [SMILES: C#Cc1cccc(Nc2ncnc3cc(OC(=O)C(C)(C)C)c(OC(=O)C(C)(C)C)cc23)c1]

[4-(3-chloro-4-fluoro-anilino)-7-(2,2-dimethylpropanoyloxy)quinazolin-6-yl] 2,2-dimethylpropanoate: The desired product was obtained as a white solid (1.0 g, 95%).

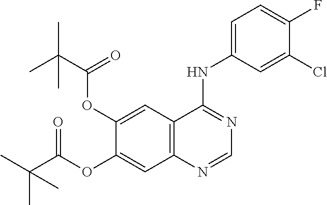 [SMILES: CC(C)(C)C(=O)Oc1cc2ncnc(Nc3ccc(F)c(Cl)c3)c2cc1OC(=O)C(C)(C)C]

[4-(3-chloro-2,4-difluoro-anilino)-7-(2,2-dimethylpropanoyloxy)quinazolin-6-yl] 2,2-dimethylpropanoate: The desired product was obtained as yellow crystals (1.6 g, quantitative).

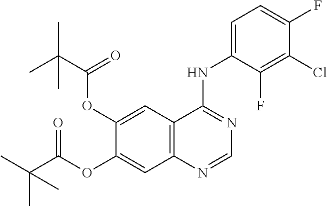 [SMILES: CC(C)(C)C(=O)Oc1cc2ncnc(Nc3ccc(F)c(Cl)c3F)c2cc1OC(=O)C(C)(C)C]

[4-(4-chloro-2-fluoro-anilino)-7-(2,2-dimethylpropanoyloxy)quinazolin-6-yl] 2,2-dimethylpropanoate: The desired product was obtained as an off-white solid (1.7 g, quantitative).

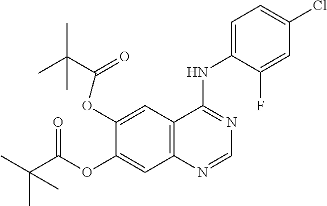 [SMILES: CC(C)(C)C(=O)Oc1cc2ncnc(Nc3ccc(Cl)cc3F)c2cc1OC(=O)C(C)(C)C]

[7-acetoxy-4-(2-chloroanilino)quinazolin-6-yl] acetate

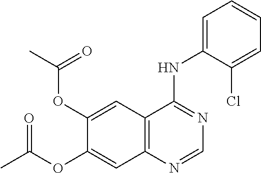 [SMILES: CC(=O)Oc1cc2ncnc(Nc3ccccc3Cl)c2cc1OC(C)=O]

To a stirred suspension of (7-acetoxy-4-chloro-quinazolin-6-yl) acetate (650 mg, 2.32 mmol) and HCl, 4 M in dioxane (1.15 mL), in DCE (7 mL) was added 2-chloroaniline (295 mg, 2.32 mmol). The resulting mixture was stirred at 80° C. for 2 h. The mixture was concentrated in vacuo then stirred in diethyl ether. The resulting suspension was filtered to collect the desired product as a beige solid (945 mg, quantitative).

¹H NMR (300 MHz, d₆-DMSO) δ 8.85 (s, 1H), 8.76 (s, 1H), 7.69-7.62 (m, 1H), 7.60-7.52 (m, 1H), 7.52-7.42 (m, 2H), 2.41 (s, 3H), 2.39 (s, 3H).

[7-acetoxy-4-[2-(trifluoromethyl)anilino]quinazolin-6-yl] acetate

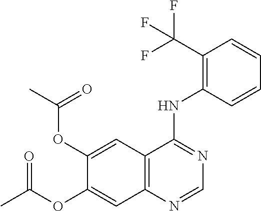 [SMILES: CC(=O)Oc1cc2ncnc(Nc3ccccc3C(F)(F)F)c2cc1OC(C)=O]

To a stirred suspension of [(7-acetoxy-4-chloro-quinazolin-6-yl) acetate (600 mg, 2.14 mmol) in DCE (10 mL) and HCl, 4 M in dioxane (1.06 mL, 4.3 mmol) was added 2-(trifluoromethyl)aniline (344 mg, 2.14 mmol) and the resulting mixture was stirred at 80° C. for 2 h then at room temperature overnight. The mixture was diluted with saturated NaHCO3. The organic phase was dried with MgSO₄, filtered and concentrated then chromatographed in 0-40% ethyl acetate in hexanes. The product was triturated with ether and hexanes to give the desired product (250 mg, 29%).

¹H NMR (300 MHz, d₆-DMSO) δ 9.87 (s, 1H), 8.44-8.36 (m, 2H), 7.88-7.72 (m, 2H), 7.68 (m, 1H), 7.64-7.51 (m, 2H), 2.38 (s, 3H), 2.35 (s, 3H).

[4-(3-chloro-2,4-difluoro-anilino)-7-methoxy-quinazolin-6-yl] 2,2-dimethylpropanoate

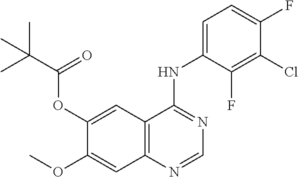 [SMILES: COc1cc2ncnc(Nc3ccc(F)c(Cl)c3F)c2cc1OC(=O)C(C)(C)C]

To a stirred suspension of (7-methoxy-4-oxo-3H-quinazolin-6-yl) 2,2-dimethylpropanoate (1.0 g, 3.62 mmol) in DCE (10 mL) was added triethylamine (1.46 g, 14.48 mmol) followed by POCl₃ (1.66 g, 10.85 mmol). The resulting mixture was stirred at 80-90° C. for 3 h. The mixture was cooled in an ice bath, quenched via addition of ice and diluted with DCM. The organic phase was separated, and the aqueous phase was re-extracted with DCM. The combined DCM extracts were washed with brine, dried, filtered and concentrated in vacuo giving a beige solid. The solid material was stirred in DCE (10 mL) and treated with 3-chloro-2,4-difluoro-aniline (0.56 g, 3.42 mmol) followed by HCl, 4 M in dioxane (1.45 mL, 7.24 mmol) and the resulting mixture was stirred at 80° C. for 30 min. The mixture was concentrated and stirred with diethyl ether. The resulting suspension was filtered to give the desired product (1.4 g, 93%).

¹H NMR (300 MHz, d₆-DMSO) δ 9.78 (s, 1H), 8.45 (s, 1H), 8.16 (s, 1H), 7.53 (s, 1H), 7.43-7.27 (m, 2H), 3.93 (s, 3H), 1.35 (s, 9H).

[4-(3-chloro-2-fluoro-anilino)-7-methoxy-quinazolin-6-yl] 2,2-dimethylpropanoate

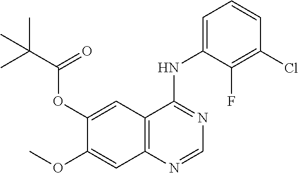 [SMILES: COc1cc2ncnc(Nc3cccc(Cl)c3F)c2cc1OC(=O)C(C)(C)C]

(4-chloro-7-methoxy-quinazolin-6-yl) 2,2-dimethylpropanoate (640 mg, 2.17 mmol) was stirred in DCE (4 mL) and treated with HCl, 4 M in dioxane (1.08 mL, 4.34 mmol) followed by 3-chloro-2-fluoro-aniline (316 mg, 2.17 mmol). The resulting mixture was stirred at 70° C. for 1 h. The mixture was concentrated in vacuo and stirred in diethyl ether giving a white suspension which was filtered to give the desired product (870 mg, quantitative).

¹H NMR (300 MHz, d₆-DMSO) δ 11.45 (brs, 1H), 8.88 (s, 1H), 8.55 (s, 1H), 7.68-7.60 (m, 1H), 7.56-7.48 (m, 1H), 7.44 (s, 1H), 7.39-7.32 (m, 1H), 4.00 (s, 3H), 1.35 (s, 9H).

[4-(3-bromo-2-fluoro-anilino)-7-(2,2-dimethylpropanoyloxy)quinazolin-6-yl] 2,2-dimethylpropanoate

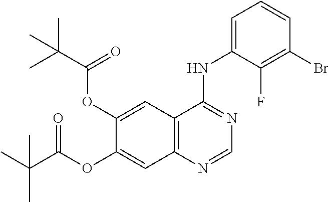 [SMILES: CC(C)(C)C(=O)Oc1cc2ncnc(Nc3cccc(Br)c3F)c2cc1OC(=O)C(C)(C)C]

¹H NMR (300 MHz, d₆-DMSO) δ 10.01 (m, 1H), 8.53 (s, 1H), 8.35 (s, 1H), 7.71 (s, 1H), 7.63 (t, J=8 Hz, 1H), 7.55 (t, J=8 Hz, 1H), 7.23 (t, J=8 Hz, 1H), 1.34 (s, 9H), 1.32 (s, 9H).

Hydrolysis of the Diester to quinazoline-6,7-diols:

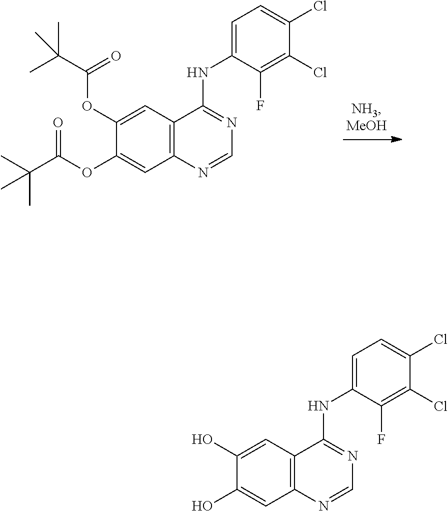 [SMILES: CC(C)(C)C(=O)Oc1cc2ncnc(Nc3ccc(Cl)c(Cl)c3F)c2cc1OC(=O)C(C)(C)C ; Oc1cc2ncnc(Nc3ccc(Cl)c(Cl)c3F)c2cc1O]

4-(3,4-dichloro-2-fluoro-anilino)quinazoline-6,7-diol:

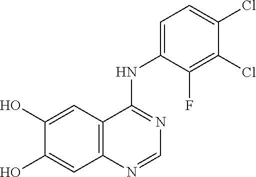 [SMILES: Oc1cc2ncnc(Nc3ccc(Cl)c(Cl)c3F)c2cc1O]

[4-(3,4-dichloro-2-fluoro-anilino)-7-(2,2-dimethylpropanoyloxy)quinazolin-6-yl] 2,2-dimethylpropanoate (8.5 g, 16.72 mmol) was stirred in methanol (150 mL). The resulting suspension was treated with ammonium hydroxide (25 mL) giving a clear solution which was stirred overnight. The mixture was concentrated in vacuo and diluted with water. The resulting suspension was filtered, the filter cake was washed with water and diethyl ether. The filter cake was dried, giving the desired product as a white solid (5.4 g, 95%). ¹H NMR (CD3OD) δ 8.27 (s, 1H), 7.66-7.57 (m, 1H), 7.54 (s, 1H), 7.42 (d, J=9 Hz, 1H), 7.07 (s, 1H).

The following compounds were made in a similar manner:

| Structure | Nomenclature | Appearance/Yield |
|---|---|---|
| 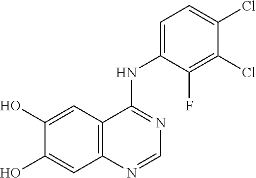 [SMILES: Oc1cc2ncnc(Nc3ccc(Cl)c(Cl)c3F)c2cc1O] | 4-(3,4-dichloro-2-fluoro- anilino)quinazoline-6,7-diol | White solid, 100% |
| 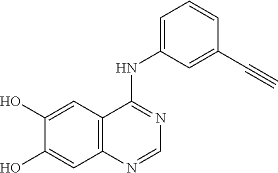 [SMILES: C#Cc1cccc(Nc2ncnc3cc(O)c(O)cc23)c1] | 4-(3- ethynylanilino)quinazoline-6,7- diol | Off-white, 95% |
| 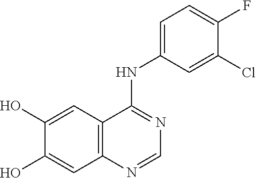 [SMILES: Oc1cc2ncnc(Nc3ccc(F)c(Cl)c3)c2cc1O] | 4-(3-chloro-4-fluoro- anilino)quinazoline-6,7-diol: The desired product was obtained as an off-white solid | White, 93% |
| 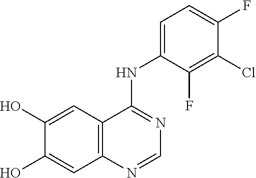 [SMILES: Oc1cc2ncnc(Nc3ccc(F)c(Cl)c3F)c2cc1O] | 4-(3-chloro-2,4-difluoro- anilino)quinazoline-6,7-diol | White solid, 93% |
| 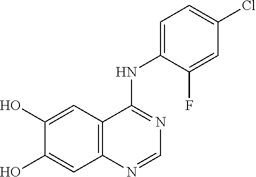 [SMILES: Oc1cc2ncnc(Nc3ccc(Cl)cc3F)c2cc1O] | 4-(3-chloro-2,4-difluoro- anilino)quinazoline-6,7-diol | White solid, 80% |
| 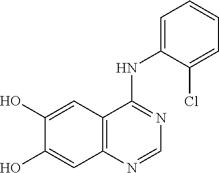 [SMILES: Oc1cc2ncnc(Nc3ccccc3Cl)c2cc1O] | 4-(2-chloroanilino)quinazoline- 6,7-diol | White solid, 86% |
| 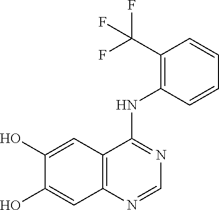 [SMILES: Oc1cc2ncnc(Nc3ccccc3C(F)(F)F)c2cc1O] | 4-[2- (trifluoromethyl)anilino]quinazoline- 6,7-diol | White solid, 95% |
| 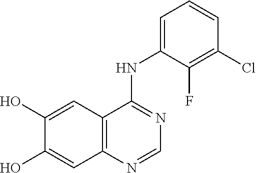 [SMILES: Oc1cc2ncnc(Nc3cccc(Cl)c3F)c2cc1O] | 4-(3-chloro-2-fluoro- anilino)quinazoline-6,7-diol | White solid, 95% |
| 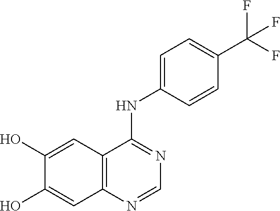 [SMILES: Oc1cc2ncnc(Nc3ccc(C(F)(F)F)cc3)c2cc1O] | 4-[4- (trifluoromethyl)anilino]quinazoline- 6,7-diol | White solid, 95% |
| 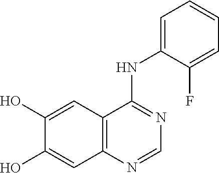 [SMILES: Oc1cc2ncnc(Nc3ccccc3F)c2cc1O] | 4-(2-fluoroanilino)quinazoline- 6,7-diol | White solid, 100% |
| 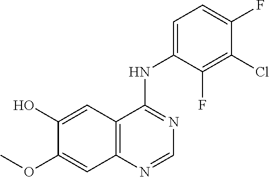 [SMILES: COc1cc2ncnc(Nc3ccc(F)c(Cl)c3F)c2cc1O] | 4-(3-chloro-2,4-difluoro- anilino)-7-methoxy-quinazolin- 6-ol | White solid, 100% |
| 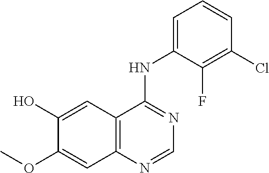 [SMILES: COc1cc2ncnc(Nc3cccc(Cl)c3F)c2cc1O] | 4-(3-chloro-2-fluoro-anilino)-7- methoxy-quinazolin-6-ol | White solid, 100% |
| 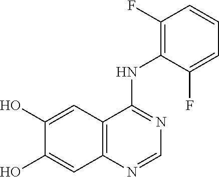 [SMILES: Oc1cc2ncnc(Nc3c(F)cccc3F)c2cc1O] | 4-(2,6- difluoroanilino)quinazoline-6,7- diol | White solid, 100% |
| 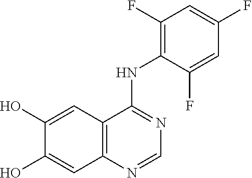 [SMILES: Oc1cc2ncnc(Nc3c(F)cc(F)cc3F)c2cc1O] | 4-(2,4,6- trifluoroanilino)quinazoline-6,7- diol | White solid, 100% |

4-(3-ethynylanilino)quinazoline-6,7-diol: The desired product was obtained as a yellow solid (0.66 g, quantitative).

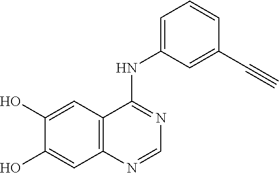 [SMILES: C#Cc1cccc(Nc2ncnc3cc(O)c(O)cc23)c1]

4-(3-chloro-4-fluoro-anilino)quinazoline-6,7-diol: The desired product was obtained as an off-white solid (0.61 g, 95%).

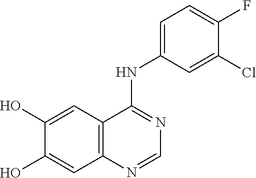 [SMILES: Oc1cc2ncnc(Nc3ccc(F)c(Cl)c3)c2cc1O]

4-(3-chloro-2,4-difluoro-anilino)quinazoline-6,7-diol: The desired product was obtained as a white solid (0.893 g, 93%).

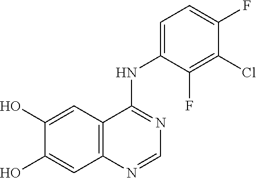 [SMILES: Oc1cc2ncnc(Nc3ccc(F)c(Cl)c3F)c2cc1O]

4-(3-chloro-2,4-difluoro-anilino)quinazoline-6,7-diol: The desired product was obtained as a white solid (0.781 g, 80%).

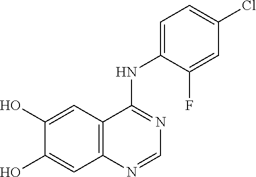 [SMILES: Oc1cc2ncnc(Nc3ccc(Cl)cc3F)c2cc1O]

4-(2-chloroanilino)quinazoline-6,7-diol:

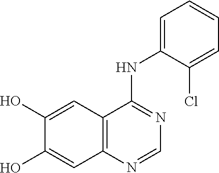 [SMILES: Oc1cc2ncnc(Nc3ccccc3Cl)c2cc1O]

To a stirred suspension of 16-99 [7-acetoxy-4-(2-chloroanilino)quinazolin-6-yl] acetate (945 mg, 2.31 mmol) in methanol (16 mL) was added concentrated ammonia (2 mL). The solid material slowly dissolved. The resulting mixture was stirred overnight (precipitate forms). The mixture was concentrated in vacuo then stirred in diethyl ether and water, then filtered to collect the desired product (570 mg, 86%).

¹H NMR (300 MHz, d₆-DMSO) δ 8.17 (s, 1H), 7.63-7.56 (m, 1H), 7.59 (s, 1H), 7.52 (d, J=9 Hz, 1H), 7.36 (t, J=9 Hz, 1H), 7.36 (d, J=9 Hz, 1H), 6.98 (s, 1H).

4-[2-(trifluoromethyl)anilino]quinazoline-6,7-diol:

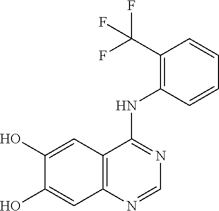 [SMILES: Oc1cc2ncnc(Nc3ccccc3C(F)(F)F)c2cc1O]

[7-acetoxy-4-[2-(trifluoromethyl)anilino]quinazolin-6-yl] acetate (240 mg) was stirred in ammonia, 2 M in methanol at room temperature overnight. The resulting mixture was concentrated in vacuo and stirred in diethyl ether. The resulting suspension was filtered to collect the desired product (180 mg, 95%).

¹H NMR (300 MHz, d₆-DMSO) δ 9.16 (brs, 1H), 8.13 (s, 1H), 7.82-7.66 (m, 2H), 7.60 (s, 1H), 7.58-7.45 (m, 2H), 7.01 (s, 1H).

4-(3-chloro-2-fluoro-anilino)quinazoline-6,7-diol:

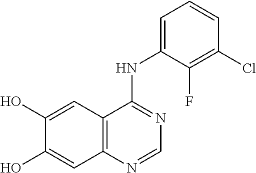 [SMILES: Oc1cc2ncnc(Nc3cccc(Cl)c3F)c2cc1O]

¹H NMR (300 MHz, d₆-DMSO) δ 8.16 (s, 1H), 7.54-7.45 (m, 2H), 7.37 (t, J=9 Hz, 1H), 7.20 (t, J=9 Hz, 1H), 6.82 (s, 1H).

4-[4-(trifluoromethyl)anilino]quinazoline-6,7-diol:

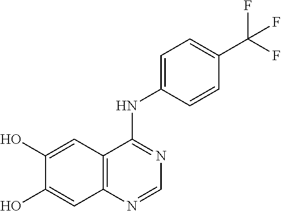 [SMILES: Oc1cc2ncnc(Nc3ccc(C(F)(F)F)cc3)c2cc1O]

¹H NMR (300 MHz, d₆-DMSO) δ 9.51 (brs, 1H), 8.40 (s, 1H), 8.12 (d, J=9 Hz, 2H), 7.73 (s, 1H), 7.66 (d, J=9 Hz, 2H), 7.25 (brs, 1H), 7.00 (s, 1H), 6.64 (brs, 1H).

4-(2-fluoroanilino)quinazoline-6,7-diol:

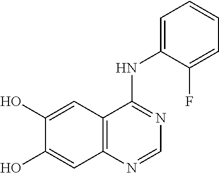 [SMILES: Oc1cc2ncnc(Nc3ccccc3F)c2cc1O]

¹H NMR (300 MHz, d₆-DMSO) δ 8.16 (s, 1H), 7.57-7.49 (m, 2H), 7.27-7.15 (m, 3H), 6.89 (s, 1H).

4-(3-chloro-2,4-difluoro-anilino)-7-methoxy-quinazolin-6-ol:

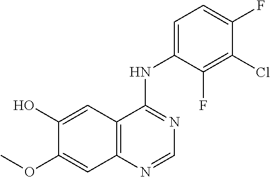 [SMILES: COc1cc2ncnc(Nc3ccc(F)c(Cl)c3F)c2cc1O]

[4-(3-chloro-2,4-difluoro-anilino)-7-methoxy-quinazolin-6-yl] 2,2-dimethylpropanoate (1.4 g, 3.32 mmol) was stirred in ammonia, 2 M in methanol (70 mL) at room temperature overnight. The mixture was concentrated in vacuo and stirred in diethyl ether and the resulting suspension was filtered to give the desired product (1.16 g, quantitative).

¹H NMR (300 MHz, d₆-DMSO) δ 11.27 (brs, 1H), 10.64 (brs, 1H), 8.77 (s, 1H), 7.96 (s, 1H), 7.65-7.54 (m, 1H), 7.50-7.42 (m, 1H), 7.39 (s, 1H), 4.01 (s, 3H).

4-(3-chloro-2-fluoro-anilino)-7-methoxy-quinazolin-6-ol:

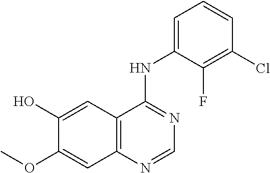 [SMILES: COc1cc2ncnc(Nc3cccc(Cl)c3F)c2cc1O]

¹H NMR (300 MHz, d₆-DMSO) δ 9.45 (s, 1H), 8.32 (s, 1H), 7.64 (s, 1H), 7.53-7.46 (m, 1H), 7.46-7.39 (m, 1H), 7.28-7.21 (m, 1H), 7.19 (s, 1H), 3.95 (s, 1H).

4-(2,6-difluoroanilino)quinazoline-6,7-diol:

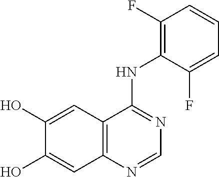 [SMILES: Oc1cc2ncnc(Nc3c(F)cccc3F)c2cc1O]

¹H NMR (400 MHz, d₆-DMSO) δ 9.18 (brs, 1H), 8.18 (s, 1H), 7.63 (s, 1H), 7.42-7.31 (m, 1H), 7.23-7.14 (m, 2H), 7.03 (s, 1H).

4-(2,4,6-trifluoroanilino)quinazoline-6,7-diol:

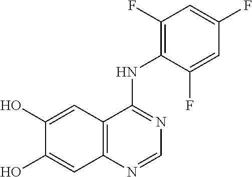 [SMILES: Oc1cc2ncnc(Nc3c(F)cc(F)cc3F)c2cc1O]

¹H NMR (400 MHz, d₆-DMSO) δ 9.14 (brs, 1H), 8.18 (s, 1H), 7.60 (s, 1H), 7.30 (t, J=10 Hz, 2H), 7.03 (s, 1H), 6.98 (brs, 1H), 6.66 (brs, 1H).

4-(3-chloro-2,4-difluoro-anilino)-7-methoxy-quinazolin-6-ol:

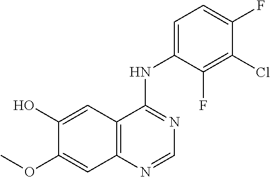 [SMILES: COc1cc2ncnc(Nc3ccc(F)c(Cl)c3F)c2cc1O]

[4-(3-chloro-2,4-difluoro-anilino)-7-methoxy-quinazolin-6-yl] 2,2-dimethylpropanoate (873 mg, 2.06 mmol) was stirred in methanol and treated with sodium hydroxide (82.8 mg, 2.06 mmol) dissolved in a minimum of water. The resulting mixture was stirred at 60 the resulting mixture was stirred at 60° C. for 30 min. The mixture was diluted with water and diethyl ether, neutralized with HCl and filtered. The solid material was stirred in diethyl ether and methanol and filtered to collect the desired product (690 mg, 98%).

¹H NMR (300 MHz, d₆-DMSO) δ 11.27 (brs, 1H), 10.64 (brs, 1H), 8.77 (s, 1H), 7.96 (s, 1H), 7.65-7.54 (m, 1H), 7.50-7.42 (m, 1H), 7.39 (s, 1H), 4.01 (s, 3H).

4-(3-ethynyl-2-fluoro-anilino)quinazoline-6,7-diol:

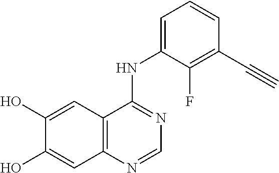 [SMILES: C#Cc1cccc(Nc2ncnc3cc(O)c(O)cc23)c1F]

To a stirred solution of [7-(2,2-dimethylpropanoyloxy)-4-[2-fluoro-3-(2-trimethylsilylethynyl)anilino]quinazolin-6-yl] 2,2-dimethylpropanoate (536 mg, 1 mmol) in methanol (20 mL) was added potassium carbonate (550 mg, 4.0 mmol) and the resulting mixture was stirred at room temperature for 1 h. The mixture was concentrated in vacuo, diluted with water and acidified to pH=6 with HCl. The suspension was filtered and the filter cake was washed with water giving the desired product (328 mg, quantitative).

¹H NMR (300 MHz, d₆-DMSO) δ 9.29 (brs, 1H), 8.23 (s, 1H), 7.64-7.51 (m, 2H), 7.43-7.34 (m, 1H), 7.21 (t, J=8 Hz, 1H), 7.03 (s, 1H), 4.49 (s, 1H).

Synthesis of 2-(difluoromethoxy)ethyl 4-methylbenzenesulfonate:

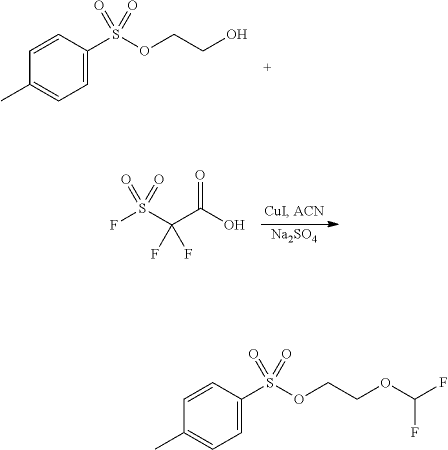

To a stirred solution of 2-hydroxyethyl 4-methylbenzenesulfonate (5.52 g, 25.5 mmol) in acetonitrile (40 mL) was added copper (I) iodide (972 mg, 5.1 mmol). The resulting mixture was stirred at 70° C. and treated with 2,2-difluoro-2-fluorosulfonyl-acetic acid as a solution in acetonitrile (5 mL) dropwise over a period of 30 min (mixture gradually turns dark red). The resulting mixture was treated with anhydrous sodium sulfate (5 mg) and stirring continued (steady evolution of gas observed, colour fades to yellow) for a further 30 min. The mixture was then cooled to room temperature, diluted with diethyl ether and washed with brine (1×), a 1:1 mixture of brine:water (2×) and brine (1×). The organic phase was dried over anhydrous sodium sulfate, filtered and concentrated in vacuo then chromatographed in 0-20% ethyl acetate in hexanes. The product containing fractions were concentrated in vacuo giving the desired product as a clear liquid (4.2 g, 62%). ¹H NMR (d₆-DMSO) δ 7.78 (d, J=9 Hz, 2H), 7.48 (d, J=9 Hz, 1H), 6.63 (t, J=75 Hz, 1H), 4.21-4.14 (m, 2H), 4.02-3.96 (m, 2H), 2.41 (s, 3H).

### Synthesis of Representative Compounds of Formula I:

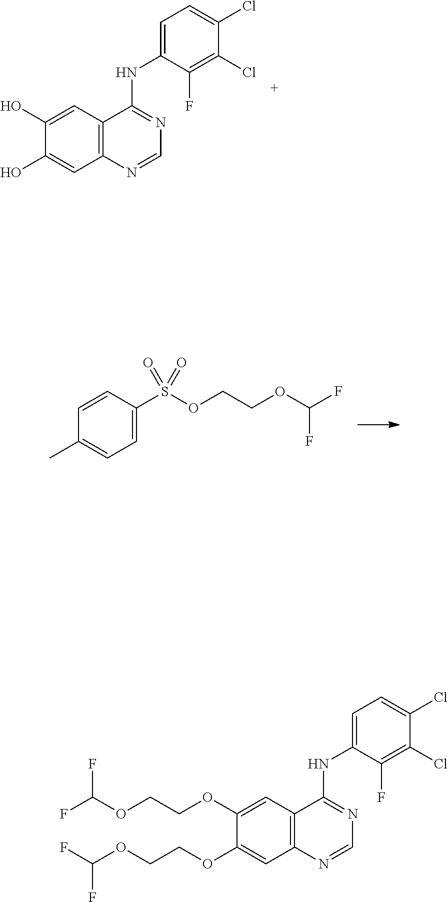 [SMILES: Oc1cc2ncnc(Nc3ccc(Cl)c(Cl)c3F)c2cc1O ; Cc1ccc(S(=O)(=O)OCCOC(F)F)cc1 ; Fc1c(Nc2ncnc3cc(OCCOC(F)F)c(OCCOC(F)F)cc23)ccc(Cl)c1Cl]

### (a) N-(3,4-dichloro-2-fluoro-phenyl)-6,7-bis[2 (difluoromethoxy)ethoxy]quinazolin-4-amine

To a stirred solution of [4-(3,4-dichloro-2-fluoro-anilino)-7-(2,2-dimethylpropanoyloxy)quinazolin-6-yl] 2,2-dimethylpropanoate (340.1 mg, 1.0 mmol) in DMF was added potassium carbonate (1.38 g, 10 mmol) followed by 2-(difluoromethoxy)ethyl 4-methylbenzenesulfonate (1.06 g, 4.0 mmol) and the resulting mixture was stirred at 60° C. for overnight. The mixture was then diluted with ethyl acetate and washed with water (3×) and brine (1×). The organic phase was dried, filtered and concentrated in vacuo then triturated with diethyl ether. The resulting suspension was filtered to collect the desired product as a pale solid (170 mg, 32%). ¹H NMR (d₆-DMSO) δ 8.47 (s, 1H), 8.31 (s, 1H), 7.45 (s, 1H), 7.43-7.31 (m, 1H), 7.14 (d, J=12 Hz, 1H), 6.38 (t, J=75 Hz, 1H), 6.36 (t, J=75 Hz, 1H), 4.41-4.20 (m, 8H) MW (MH⁺): 529.3.

Hydrochloride salt: ¹H NMR (d₆-DMSO) δ 11.88 (brs, 1H), 8.84 (s, 1H), 8.45 (s, 1H), 7.71-7.55 (m, 2H), 7.40 (s, 1H), 6.76 (2t, J=75 Hz, 1H), 4.48-4.39 (m, 4H), 4.32-4.24 (m, 4H).

The following compounds were made in a similar manner:

### (b): 6,7-bis[2-(difluoromethoxy)ethoxy]-N-(3-ethynylphenyl)quinazolin-4-amine

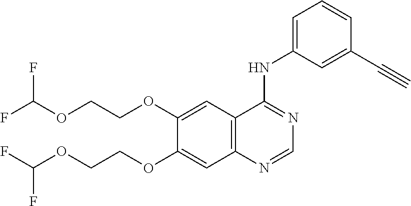 [SMILES: C#Cc1cccc(Nc2ncnc3cc(OCCOC(F)F)c(OCCOC(F)F)cc23)c1]

White solid, 50%. ¹H NMR (d₆-DMSO) (9.49 (s, 1H), 8.50 (s, 1H), 7.99-7.84 (m, 3H), 7.39 (t, J=7.5 Hz, 1H), 7.26 (s, 1H), 7.20 (d, J=6 Hz, 1H), 6.77 (t, J=75 Hz, 1H), 6.76 (t, J=75 Hz, 1H), 4.41-4.33 (m, 4H), 4.31-4.20 (m, 4H), 4.19 (s, 1H), MW (MH⁺): 466.4.

### (c): N-(3-chloro-4-fluoro-phenyl)-6,7-bis[2-(difluoromethoxy)ethoxy]quinazolin-4-amine

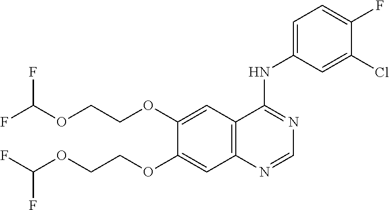 [SMILES: Fc1ccc(Nc2ncnc3cc(OCCOC(F)F)c(OCCOC(F)F)cc23)cc1Cl]

(White solid 55%). ¹H NMR (d₆-DMSO) (9.55 (s, 1H), 8.50 (s, 1H), 8.11 (dd, J=9 Hz, 3 Hz, 1H), 7.88 (s, 1H), 7.80-7.74 (m, 1H), 7.44 (t, J=9 Hz, 1H), 7.26 (m, 1H), 6.77 (t, J=75 Hz, 1H), 6.75 (t, J=75 Hz, 1H), 4.41-4.43 (m, 4H), 4.30-4.21 (m, 4H), MW (MH+): 494.8.

### (d): N-(3-chloro-2,4-difluoro-phenyl)-6,7-bis[2-(difluoromethoxy)ethoxy]quinazolin-4-amine

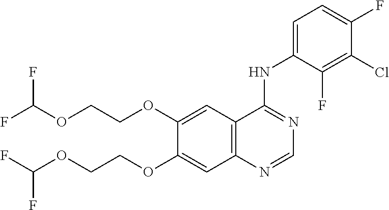 [SMILES: Fc1ccc(Nc2ncnc3cc(OCCOC(F)F)c(OCCOC(F)F)cc23)c(F)c1Cl]

(White solid, 20%). ¹H NMR (CDCl₃) (8.50 (s, 1H), 8.28 (s, 1H), 7.52 (s, 1H), 7.45-7.31 (m, 1H), 6.94-6.83 (m, 1H), 6.37 (t, J=75 Hz, 1H), 6.34 (t, J=75 Hz, 1H), 4.36-4.22 (m, 8H), MW (MH+): 512.80.

### (e): N-(4-chloro-2-fluoro-phenyl)-6,7-bis[2-(difluoromethoxy)ethoxy]quinazolin-4-amine

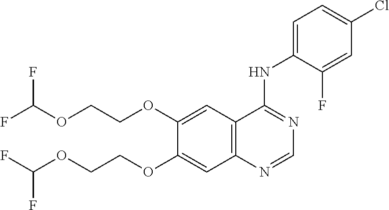 [SMILES: Fc1cc(Cl)ccc1Nc1ncnc2cc(OCCOC(F)F)c(OCCOC(F)F)cc12]

(White solid). ¹H NMR (d₆-DMSO) δ 9.53 (s, 1H), 8.36 (s, 1H), 7.85 (s, 1H), 7.62-7.50 (m, 2H), 7.36-7.30 (m, 1H), 7.25 (s, 1H), 6.76 (t, J=76 Hz, 1H), 6.75 (t, J=75 Hz, 1H), 4.40-4.30 (m, 4H), 4.30-4.20 (m, 4H), MW (MH+): 494.81.

In a like manner, the following additional compounds of the application were prepared.

### (g): N-(3-chloro-2-fluoro-phenyl)-6,7-bis[2-(difluoromethoxy)ethoxy]quinazolin-4-amine

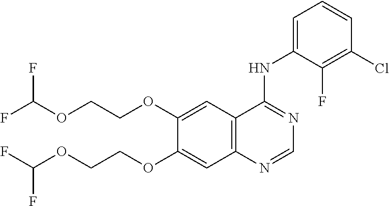 [SMILES: Fc1c(Cl)cccc1Nc1ncnc2cc(OCCOC(F)F)c(OCCOC(F)F)cc12]

(White solid, 52%). ¹H NMR (400 MHz, d₆-DMSO) δ 9.60 (s, 1H), 8.38 (s, 1H), 7.82 (s, 1H), 7.55-7.43 (m, 2H), 7.27 (t, J=8 Hz, 1H), 7.22 (s, 1H), 6.78 (t, J=76 Hz, 1H), 4.36-4.30 (m, 2H), 4.30-4.26 (m, 2H), 3.94 (s, 3H). MW (MH+): 494.8.

### (i): N-(3-chloro-2-fluoro-phenyl)-6-[2-(difluoromethoxy)ethoxy]-7-methoxy-quinazolin-4-amine

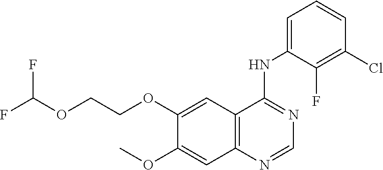 [SMILES: COc1cc2ncnc(Nc3cccc(Cl)c3F)c2cc1OCCOC(F)F]

(White solid, 56%). ¹H NMR (400 MHz, d₆-DMSO) δ 9.60 (s, 1H), 8.38 (s, 1H), 7.82 (s, 1H), 7.55-7.43 (m, 2H), 7.27 (t, J=8 Hz, 1H), 7.22 (s, 1H), 6.78 (t, J=76 Hz, 1H), 4.36-4.30 (m, 2H), 4.30-4.26 (m, 2H), 3.94 (s, 3H). MW (MH+): 414.8.

### (K): N-(3-chloro-2,4-difluoro-phenyl)-6-[2-(difluoromethoxy)ethoxy]-7-methoxy-quinazolin-4-amine

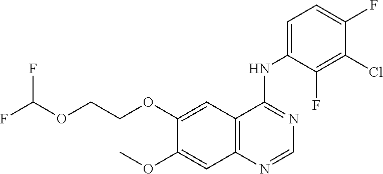 [SMILES: COc1cc2ncnc(Nc3ccc(F)c(Cl)c3F)c2cc1OCCOC(F)F]

To a stirred suspension of 4-(3-chloro-2,4-difluoro-anilino)-7-methoxy-quinazolin-6-ol (580 mg, 1.72 mmol) and potassium carbonate (710 mg, 5.15 mmol) in DMF (10 mL) at 80° C. was added 2-(difluoromethoxy)ethyl 4-methylbenzenesulfonate (686 mg, 2.58 mmol) and the resulting mixture was stirred for 3 h. The resulting mixture was diluted with ethyl acetate and washed with water (3×) and brine (1×). The organic phase was dried, filtered and concentrated then chromatographed in 50-100% ethyl acetate in hexanes. The product containing fractions were triturated with diethyl ether and hexanes giving the desired product as white solid (413 mg, 55%).

(White solid, 56%). ¹H NMR (400 MHz, d₆-DMSO) δ 9.60 (s, 1H), 8.37 (s, 1H), 7.81 (s, 1H), 7.60-7.51 (m, 1H), 7.38 (td, J=8 Hz, 4 Hz, 1H), 7.21 (s, 1H), 6.78 (t, J=76 Hz, 1H), 4.35-4.30 (m, 2H), 4.30-4.24 (m, 2H), 3.94 (s, 3H). MW (MH+): 432.8.

### (Q): N-(3-chloro-2,4-difluoro-phenyl)-6-[3-[4-(difluoromethoxy)-1-piperidyl]propoxy]-7-methoxy-quinazolin-4-amine

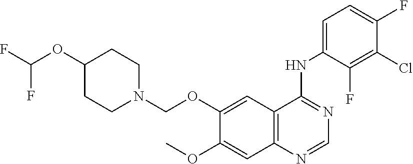 [SMILES: COc1cc2ncnc(Nc3ccc(F)c(Cl)c3F)c2cc1OCN1CCC(OC(F)F)CC1]

(White solid, 41%). ¹H NMR (400 MHz, d₆-DMSO) δ 9.61 (s, 1H), 8.34 (s, 1H), 7.76 (s, 1H), 7.61-7.46 (m, 1H), 7.41-7.32 (m, 1H), 7.18 (s, 1H), 6.69 (t, J=76 Hz, 1H), 4.20-4.02 (m, 2H), 4.00-3.82 (m, 1H), 2.78-2.61 (m, 2H), 2.56-2.36 (m, 2H), 2.25-2.05 (m, 2H), 2.05-1.91 (m, 2H), 1.91-1.76 (m, 2H), 1.65-1.52 (m, 2H). MW (MH+): 530.0.

### (W): [4-(3-chloro-2-fluoro-anilino)-7-methoxy-quinazolin-6-yl] 4-(difluoromethoxymethyl)piperidine-1-carboxylate

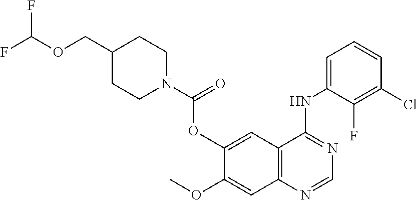 [SMILES: COc1cc2ncnc(Nc3cccc(Cl)c3F)c2cc1OC(=O)N1CCC(COC(F)F)CC1]

¹H NMR (300 MHz, d₆-DMSO) δ 9.72 (s, 1H), 8.45 (s, 1H), 8.19 (s, 1H), 7.55-7.42 (m, 2H), 7.31 (s, 1H), 7.30-7.21 (m, 1H), 6.67 (t, J=76 Hz, 1H), 4.29-4.13 (m, 1H), 4.08-3.95 (m, 1H), 3.93 (s, 3H), 3.75 (d, J=6 Hz, 2H), 3.18-3.00 (m, 1H), 2.99-2.80 (m, 1H), 1.99-1.82 (m, 1H), 1.82-1.66 (m, 2H), 1.38-1.10 (m, 2H).

### (X): [4-(3-chloro-2,4-difluoro-anilino)-7-methoxy-quinazolin-6-yl] 4-(difluoromethoxy)piperidine-1-carboxylate

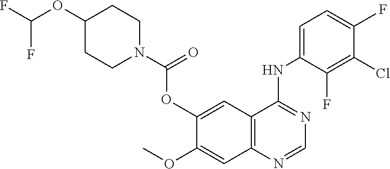 [SMILES: COc1cc2ncnc(Nc3ccc(F)c(Cl)c3F)c2cc1OC(=O)N1CCC(OC(F)F)CC1]

(White solid, 20%). ¹H NMR (300 MHz, d₆-DMSO) δ 9.73 (s, 1H), 8.45 (s, 1H), 8.18 (s, 1H), 7.59-7.49 (m, 1H), 7.41-7.32 (m, 1H), 7.32 (s, 1H), 6.78 (t, J=76 Hz, 1H), 4.49-4.36 (m, 1H), 3.93 (s, 3H), 3.97-3.82 (m, 1H), 3.79-3.61 (m, 1H), 3.57-3.25 (m, 2H), 2.05-1.87 (m, 2H), 1.76-1.51 (m, 2H). MW (MH+): 515.9.

### (Bb): [4-(3-chloro-2,4-difluoro-anilino)-7-methoxy-quinazolin-6-yl] 3-(difluoromethoxy)azetidine-1-carboxylate

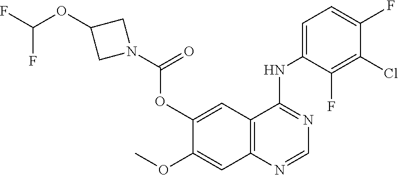 [SMILES: COc1cc2ncnc(Nc3ccc(F)c(Cl)c3F)c2cc1OC(=O)N1CC(OC(F)F)C1]

(White solid, 3%). ¹H NMR (300 MHz, d₆-DMSO) δ 10.17 (brs, 1H), 8.55 (s, 1H), 8.26 (s, 1H), 7.61-7.48 (m, 1H), 7.45-7.34 (m, 1H), 7.33 (s, 1H), 6.79 (t, J=74 Hz, 1H), 5.13-5.00 (m, 1H), 4.59-4.43 (m, 1H), 4.43-4.27 (m, 1H), 4.27-4.11 (m, 1H), 4.02-3.90 (m, 1H), 3.96 (s, 3H).

### (Dd): [4-(2-chloroanilino)-7-(2,2-dimethylpropanoyloxy)quinazolin-6-yl] 2,2-dimethylpropanoate

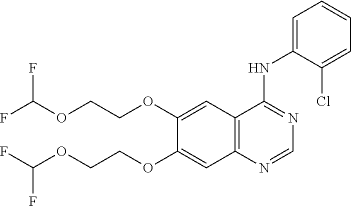 [SMILES: FC(F)OCCOc1cc2ncnc(Nc3ccccc3Cl)c2cc1OCCOC(F)F]

To a stirred suspension of [4-(2-chloroanilino)-7-(2,2-dimethylpropanoyloxy)quinazolin-6-yl] 2,2-dimethylpropanoate (567 mg, 1.97 mmol) and potassium carbonate (1.36 g, 9.85 mmol) in DMF (6 mL) at 80° C. was added 2-(difluoromethoxy)ethyl 4-methylbenzenesulfonate (1.31 g, 4.93 mmol). The mixture was stirred at 80° C. for 2 h then at room temperature overnight. The mixture was diluted with ethyl acetate and diethyl ether and washed with brine (2×), water (1×) and brine (1×). The organic phase was dried, filtered and concentrated in vacuo then chromatographed in 0-100% ethyl acetate in hexanes. The product containing fractions were concentrated and stirred in hexanes giving the desired product as a white solid (250 mg, 26%).

¹H NMR (300 MHz, d₆-DMSO) δ 8.76-8.68 (m, 2H), 7.74 (brs, 1H), 7.46 (t, J=9 Hz, 1H), 7.38 (t, J=9 Hz, 1H), 7.28 (s, 1H), 7.20 (s, 1H), 7.08 (t, J=9 Hz, 1H), 6.41 (t, J=74 Hz, 1H), 6.39 (t, J=75 Hz, 1H), 4.40-4.35 (m, 4H), 4.35-4.29 (m, 4H). MW (MH+): 476.8.

### (Ee): 6,7-bis[2-(difluoromethoxy)ethoxy]-N-[2-(trifluoromethyl)phenyl]quinazolin-4-amine

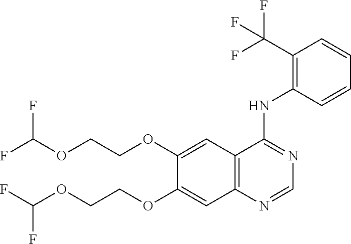 [SMILES: FC(F)OCCOc1cc2ncnc(Nc3ccccc3C(F)(F)F)c2cc1OCCOC(F)F]

To a stirred suspension of 4-[2-(trifluoromethyl)anilino]quinazoline-6,7-diol (177 mg, 0.551 mmol) and potassium carbonate (380 mg, 2.76 mmol) in DMF (2 mL) was added 2-(difluoromethoxy)ethyl 4-methylbenzenesulfonate (366 mg, 1.38 mmol) and the resulting mixture was stirred at 60° C. for 2 h then at room temperature overnight. The mixture was diluted with ethyl acetate and diethyl ether and washed with brine (2×), water (1×) and brine (1×). The organic phase was dried, filtered and concentrated in vacuo then chromatographed in 25-75% ethyl acetate in hexanes. The product containing fractions were concentrated in vacuo and stirred in hexanes/diethyl ether. The resulting suspension was filtered to collect the desired product as a white solid (93 mg, 33%).

¹H NMR (300 MHz, d₆-DMSO) δ 9.48 (s, 1H), 8.26 (s, 1H), 7.90 (s, 1H), 7.82 (d, J=6 Hz, 1H), 7.75 (t, J=6 Hz, 1H), 7.59-7.51 (m, 2H), 7.23 (s, 1H), 6.77 (t, J=76 Hz, 1H), 6.76 (t, J=76 Hz, 1H), 4.40-4.29 (m, 4H), 4.29-4.20 (m, 4H). MW (MH+): 510.4.

### (Ff): N-(3-chloro-2-fluoro-phenyl)-6,7-bis[2-(difluoromethoxy)ethoxy]quinazolin-4-amine

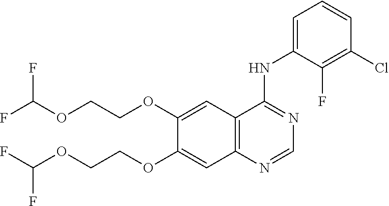 [SMILES: Fc1c(Cl)cccc1Nc1ncnc2cc(OCCOC(F)F)c(OCCOC(F)F)cc12]

(White solid, 42%). ¹H NMR (300 MHz, d₆-DMSO) δ 9.68 (s, 1H), 8.38 (s, 1H), 7.86 (s, 1H), 7.57-7.43 (m, 2H), 7.31-7.22 (m, 2H), 6.77 (t, J=75 Hz, 1H), 6.76 (t, J=75 Hz, 1H), 4.41-4.31 (m, 4H), 4.30-4.21 (m, 4H). MW (MH+): 494.9.

### (Gg): 6,7-bis[2-(difluoromethoxy)ethoxy]-N-[4-(trifluoromethyl)phenyl]quinazolin-4-amine

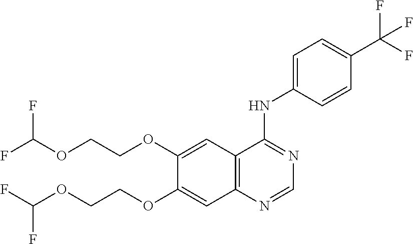 [SMILES: FC(F)OCCOc1cc2ncnc(Nc3ccc(C(F)(F)F)cc3)c2cc1OCCOC(F)F]

(White solid, 35%). ¹H NMR (300 MHz, d₆-DMSO) δ 9.71 (s, 1H), 8.55 (s, 1H), 8.08 (d, J=9 Hz, 2H), 7.94 (s, 1H), 7.74 (d, J=9 Hz, 2H), 7.29 (s, 1H), 6.77 (t, J=76 Hz, 1H), 6.76 (t, J=76 Hz, 1H), 4.42-4.33 (m, 4H), 4.32-4.20 (m, 4H). MW (MH+): 510.3.

### (Hh): 6,7-bis[2-(difluoromethoxy)ethoxy]-N-(2-fluorophenyl)quinazolin-4-amine

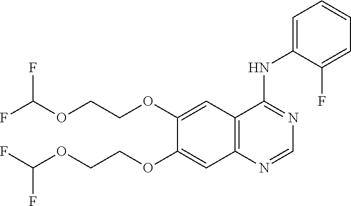 [SMILES: Fc1ccccc1Nc1ncnc2cc(OCCOC(F)F)c(OCCOC(F)F)cc12]

(White solid, 53%). ¹H NMR (300 MHz, d₆-DMSO) δ 9.48 (s, 1H), 8.33 (s, 1H), 7.87 (s, 1H), 7.58-7.47 (m, 1H), 7.35-7.16 (m, 4H), 6.77 (t, J=76 Hz, 1H), 6.76 (t, J=76 Hz, 1H), 4.42-4.30 (m, 4H), 4.30-4.19 (m, 4H). MW (MH+): 460.4.

### (Kk): 6,7-bis[2-(difluoromethoxy)ethoxy]-N-(2,6-difluorophenyl)quinazolin-4-amine

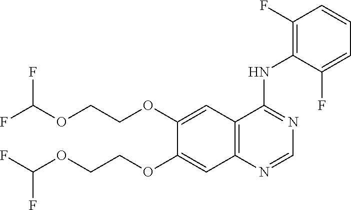 [SMILES: Fc1cccc(F)c1Nc1ncnc2cc(OCCOC(F)F)c(OCCOC(F)F)cc12]

¹H NMR (400 MHz, d₆-DMSO) δ 9.46 (s, 1H), 8.33 (s, 1H), 7.89 (s, 1H), 7.46-7.34 (m, 1H), 7.30-7.17 (m, 3H), 6.77 (t, J=76 Hz, 1H), 6.76 (t, J=76 Hz, 1H), 4.40-4.30 (m, 4H), 4.30-4.19 (m, 4H).

### (Ll): 6,7-bis[2-(difluoromethoxy)ethoxy]-N-(2,4,6-trifluorophenyl)quinazolin-4-amine

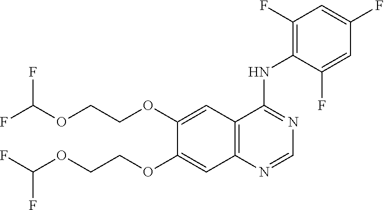 [SMILES: Fc1cc(F)c(Nc2ncnc3cc(OCCOC(F)F)c(OCCOC(F)F)cc23)c(F)c1]

¹H NMR (400 MHz, d₆-DMSO) δ 9.42 (s, 1H), 8.34 (s, 1H), 7.86 (s, 1H), 7.35 (t, J=8 Hz, 2H), 7.26 (s, 1H), 6.77 (t, J=76 Hz, 1H), 6.75 (t, J=76 Hz, 1H), 4.40-4.29 (m, 4H), 4.30-4.20 (m, 4H).

### (Mm): N-(3-chloro-2,4-difluoro-phenyl)-6-[3-[4-(difluoromethoxy)-1-piperidyl]propoxy]-7-methoxy-quinazolin-4-amine

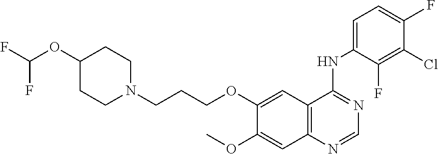 [SMILES: COc1cc2ncnc(Nc3ccc(F)c(Cl)c3F)c2cc1OCCCN1CCC(OC(F)F)CC1]

4-(3-chloro-2,4-difluoro-anilino)-7-methoxy-quinazolin-6-ol (94.2 mg, 0.279 mmol), 1-(3-bromopropyl)-4-(difluoromethoxy)piperidine (114 mg, 0.419 mmol) and potassium carbonate (116 mg, 0.838 mmol) were stirred in DMF at 80° C. for 2 h. The mixture was diluted with ethyl acetate and washed with brine (1×), water (1×) and brine (1×). The organic phase was dried, filtered and concentrated in vacuo then chromatographed in 0-50% THF in ethyl acetate. The product containing fractions were concentrated and stirred in hexanes and the resulting suspension was filtered to collect the desired product as a white solid (60 mg, 41%).

¹H NMR (400 MHz, d₆-DMSO) δ 9.61 (s, 1H), 8.34 (s, 1H), 7.76 (s, 1H), 7.61-7.46 (m, 1H), 7.41-7.32 (m, 1H), 7.18 (s, 1H), 6.69 (t, J=76 Hz, 1H), 4.20-4.02 (m, 2H), 4.00-3.82 (m, 1H), 2.78-2.61 (m, 2H), 2.56-2.36 (m, 2H), 2.25-2.05 (m, 2H), 2.05-1.91 (m, 2H), 1.91-1.76 (m, 2H), 1.65-1.52 (m, 2H).

### (Nn): (6,7-bis[2-(difluoromethoxy)ethoxy]-N-(3-ethynyl-2-fluoro-phenyl)quinazolin-4-amine

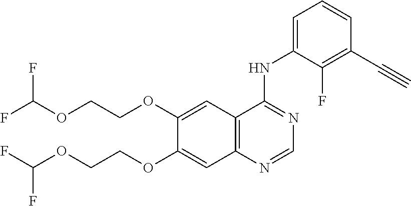 [SMILES: C#Cc1cccc(Nc2ncnc3cc(OCCOC(F)F)c(OCCOC(F)F)cc23)c1F]

¹H NMR (400 MHz, d₆-DMSO) δ 9.59 (s, 1H), 8.40 (s, 1H), 7.89 (s, 1H), 7.66-7.59 (m, 1H), 7.50-7.43 (m, 1H), 7.32-7.25 (m, 2H), 6.80 (t, J=76 Hz, 1H), 6.79 (t, J=76 Hz, 1H), 4.55 (s, 1H), 4.45-4.34 (m, 4H), 4.34-4.24 (m, 4H).

### (Oo): [4-(3-chloro-2,4-difluoro-anilino)-7-methoxy-quinazolin-6-yl] 4-(difluoromethoxy)piperidine-1-carboxylate

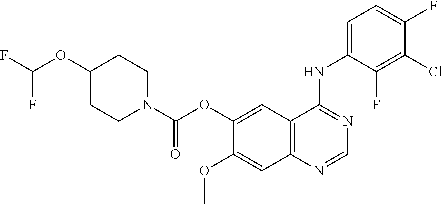 [SMILES: COc1cc2ncnc(Nc3ccc(F)c(Cl)c3F)c2cc1OC(=O)N1CCC(OC(F)F)CC1]

To a stirred solution of 4-(difluoromethoxy)piperidine hydrochloride (89 mg, 0.474 mmol) in DCM was added triphosgene (140.7 mg, 0.474 mmol). The resulting mixture was stirred at −78° C. under nitrogen and treated with pyridine (150 mg, 1.90 mmol). The mixture was stirred at 0° C. warmed slowly to room temperature then stirred at room temperature overnight. The mixture was concentrated in vacuo then mixed with 4-(3-chloro-2,4-difluoro-anilino)-7-methoxy-quinazolin-6-ol (160 mg, 0.474 mmol) and potassium carbonate (131 mg, 0.948 mmol) in DMF (5 mL) and stirred at room temperature overnight. The mixture was diluted with ethyl acetate and washed with brine (1×), water (1×) and brine (1×). The organic phase was dried, filtered and concentrated in vacuo then chromatographed in 0-70% ethyl acetate in hexanes. The product containing fractions were concentrated in vacuo and triturated with hexanes to give the desired product as a white solid (50 mg, 20%).

¹H NMR (300 MHz, d₆-DMSO) δ 9.73 (s, 1H), 8.45 (s, 1H), 8.18 (s, 1H), 7.59-7.49 (m, 1H), 7.41-7.32 (m, 1H), 7.32 (s, 1H), 6.78 (t, J=76 Hz, 1H), 4.49-4.36 (m, 1H), 3.93 (s, 3H), 3.97-3.82 (m, 1H), 3.79-3.61 (m, 1H), 3.57-3.25 (m, 2H), 2.05-1.87 (m, 2H), 1.76-1.51 (m, 2H).

### (Pp): [4-(3-chloro-2,4-difluoro-anilino)-7-methoxy-quinazolin-6-yl] (3S)-3-(difluoromethoxy)piperidine-1-carboxylate

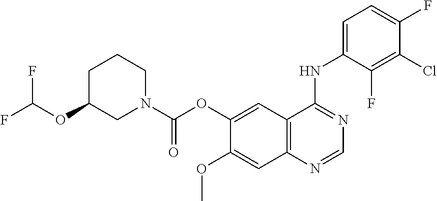 [SMILES: COc1cc2ncnc(Nc3ccc(F)c(Cl)c3F)c2cc1OC(=O)N1CCC[C@H](OC(F)F)C1]

¹H NMR (300 MHz, d₆-DMSO) δ 9.75 (s, 1H), 8.44 (s, 1H), 8.17 (s, 1H), 7.60-7.49 (m, 1H), 7.42-7.32 (m, 1H), 7.32 (s, 1H), 6.79 (t, J=75 Hz, 1H), 4.35-4.19 (m, 1H), 3.93 (s, 1H), 3.85-3.55 (m, 3H), 3.54-3.35 (m, 1H), 2.03-1.87 (m, 1H), 1.87-1.67 (m, 2H), 1.66-1.43 (m, 1H), MS: 515.6 (MH+).

### (Qq): [4-(3-chloro-2,4-difluoro-anilino)-7-methoxy-quinazolin-6-yl] (3R)-3-(difluoromethoxymethyl)pyrrolidine-1-carboxylate

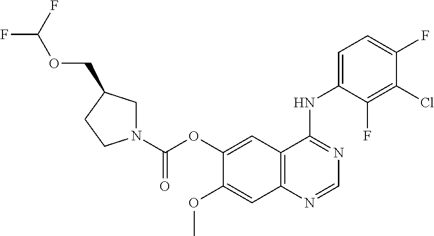 [SMILES: COc1cc2ncnc(Nc3ccc(F)c(Cl)c3F)c2cc1OC(=O)N1CC[C@@H](COC(F)F)C1]

¹H NMR (300 MHz, d₆-DMSO) δ 9.74 (s, 1H), 8.45 (s, 1H), 8.19 (s, 1H), 7.60-7.50 (m, 1H), 7.42-7.31 (m, 1H), 7.33 (s, 1H), 6.70 (t, J=75 Hz, 1H), 4.28-4.06 (m, 1H), 4.06-3.93 (m, 2H), 3.93 (s, 1H), 3.66-3.50 (m, 1H), 3.45-3.33 (m, 1H), 2.17-1.82 (m, 4H), MS: 515.7 (MH+).

### (Rr): [4-(3-chloro-2-fluoro-anilino)-7-methoxy-quinazolin-6-yl] 3-(difluoromethoxy)azetidine-1-carboxylate

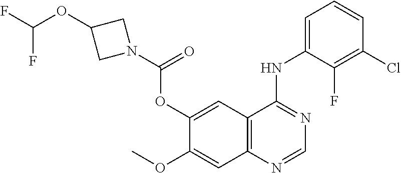 [SMILES: COc1cc2ncnc(Nc3cccc(Cl)c3F)c2cc1OC(=O)N1CC(OC(F)F)C1]

(White solid, 3%). ¹H NMR (300 MHz, d₆-DMSO) δ 9.74 (s, 1H), 8.45 (s, 1H), 8.21 (s, 1H), 7.55-7.41 (m, 2H), 7.32 (s, 1H), 7.30-7.21 (m, 1H), 6.79 (t, J=74 Hz, 1H), 5.12-5.01 (m, 1H), 4.60-4.44 (m, 1H), 4.43-4.25 (m, 1H), 4.24-4.07 (m, 1H), 4.07-3.95 (m, 1H), 3.94 (s, 3H).

### N-(3-ethynylphenyl)-6,7-bis(2-methoxyethoxy)quinazolin-4-amine (Erlotinib)

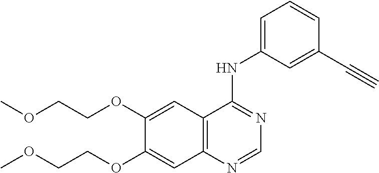 [SMILES: C#Cc1cccc(Nc2ncnc3cc(OCCOC)c(OCCOC)cc23)c1]

To a stirred solution of 4-(3-ethynylanilino)quinazoline-6,7-diol (0.7 g, 2.52 mmol), PPh₃ (2.64 g, 10.10 mmol) and 2-methoxyethanol (10.10 mmol) in THF cooled to 0° C. was added DEAD (10.10 mmol) slowly. The resulting mixture was warmed to room temperature and stirred overnight. The mixture was then diluted with ethyl acetate and washed with brine, water and brine. The organic phase was dried, filtered and concentrated in vacuo then chromatographed in 0-100% ethyl acetate in hexanes giving the desired product (550 mg, 55%) as a white solid.

¹H NMR (CDCl₃, 400 MHz) δ 8.64 (s, 1H), 7.90-7.87 (m, 1H), 7.55-7.51 (m, 1H), 7.41 (s, 1H), 7.19-7.13 (m, 3H), 4.27-4.21 (m, 4H), 3.83-3.80 (m, 4H), 3.45 (s, 3H), 3.44 (s, 3H). MH⁺ 394.2.

Table 1 provides a summary of the LCMS characterization of the representative compounds of Formula I.

### Example 3: Representative Synthesis of Compounds of Formula I, Wherein X¹ is NH

### 4-chloro-7-fluoro-6-nitro-quinazoline

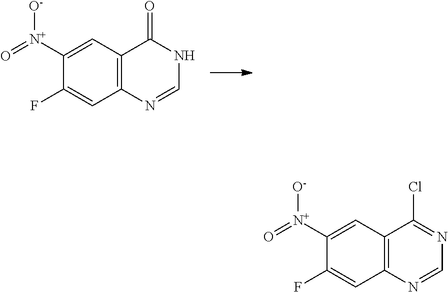 [SMILES: O=c1[nH]cnc2cc(F)c([N+](=O)[O-])cc12 ; O=[N+]([O-])c1cc2c(Cl)ncnc2cc1F]

7-fluoro-6-nitro-3H-quinazolin-4-one (5 g, 23.91 mmol) was stirred in SOCl2 (50 mL) and treated with DMF (1 drop). The resulting mixture was stirred at reflux temperature for 3 h, then concentrated in vacuo giving the crude product as a pale yellow solid (used directly in the subsequent reaction).

### N-(3-chloro-2,4-difluoro-phenyl)-7-fluoro-6-nitro-quinazolin-4-amine hydrochloride

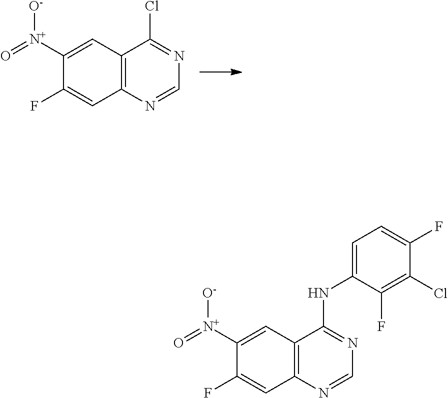 [SMILES: O=[N+]([O-])c1cc2c(Cl)ncnc2cc1F ; O=[N+]([O-])c1cc2c(Nc3ccc(F)c(Cl)c3F)ncnc2cc1F]

To a stirred suspension of 4-chloro-7-fluoro-6-nitro-quinazoline (5.4 g, 23.72 mmol) in DCM (50 mL) was added 3-chloro-2,4-difluoro-aniline (4.27 g, 26.10 mmol) as a solution in iPrOH (50 mL). The resulting mixture was stirred at room temperature for 30 min (mild exotherm observed). The mixture was concentrated to near dryness and stirred in diethyl ether. The resulting suspension was filtered to collect the desired product as a pale yellow solid (9.3 g, quantitative).

### N-(3-chloro-2,4-difluoro-phenyl)-7-methoxy-6-nitro-quinazolin-4-amine

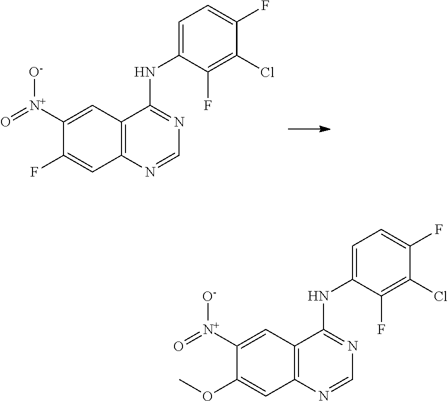 [SMILES: O=[N+]([O-])c1cc2c(Nc3ccc(F)c(Cl)c3F)ncnc2cc1F ; COc1cc2ncnc(Nc3ccc(F)c(Cl)c3F)c2cc1[N+](=O)[O-]]

To a stirred suspension of N-(3-chloro-2,4-difluoro-phenyl)-7-fluoro-6-nitro-quinazolin-4-amine hydrochloride in MeOH (50 mL) was added sodium methoxide (3.32 g, 61.36 mmol) and the resulting mixture was stirred at reflux for 1 h. The mixture was concentrated in vacuo, stirred in H2O and neutralized with HCl. The mixture was stirred at room temperature then filtered to collect the desired product as a pale yellow solid (3.7 g, quantitative).

### N4-(3-chloro-2,4-difluoro-phenyl)-7-methoxy-quinazoline-4,6-diamine

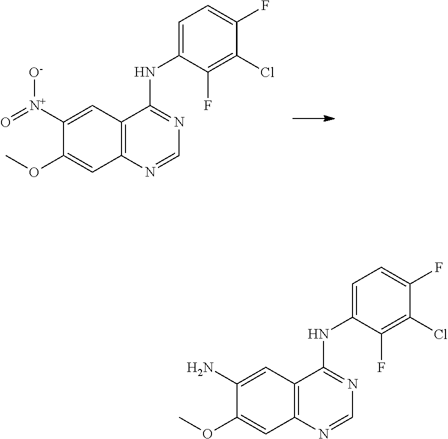 [SMILES: COc1cc2ncnc(Nc3ccc(F)c(Cl)c3F)c2cc1[N+](=O)[O-] ; COc1cc2ncnc(Nc3ccc(F)c(Cl)c3F)c2cc1N]

To a stirred solution of N-(3-chloro-2,4-difluoro-phenyl)-7-methoxy-6-nitro-quinazolin-4-amine (3.7 g, 10.09 mmol) in THF was added Raney Nickel (1.0 g) and the resulting mixture was stirred overnight at room temperature under an atmosphere of hydrogen (balloon pressure). The mixture was filtered and concentrated in vacuo then triturated with diethyl ether and hexanes to give the desired product (3.26 g, 96%).

### (A): N-[4-(3-chloro-2,4-difluoro-anilino)-7-methoxy-quinazolin-6-yl]-4-(difluoromethoxymethyl)piperidine-1-carboxamide

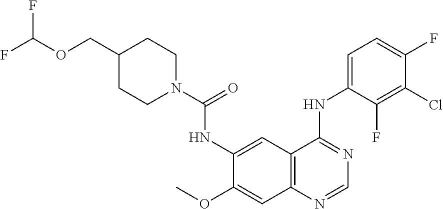 [SMILES: COc1cc2ncnc(Nc3ccc(F)c(Cl)c3F)c2cc1NC(=O)N1CCC(COC(F)F)CC1]

To a stirred solution of N4-(3-chloro-2,4-difluoro-phenyl)-7-methoxy-quinazoline-4,6-diamine (165 mg, 0.49 mmol) and pyridine (193 mg, 2.45 mmol) in DMF (3 mL) was added phenyl chloroformate (230 mg, 0.735 mmol) and the resulting mixture was stirred at 70° C. for 2 h. The mixture was cooled to room temperature, treated with 4-(difluoromethoxymethyl)piperidine hydrochloride (130 mg, 0.644 mmol) and stirred at 70° C. for 2 h. The mixture was diluted with ethyl acetate and washed with brine (1×), water (1×) and brine (1×). The organic phase was dried, filtered and concentrated in vacuo then chromatographed in 50-100% ethyl acetate in hexanes. The product containing fractions were concentrated in vacuo and triturated with diethyl ether and hexanes, giving the desired product as a pale orange solid (10 mg, 4%).

¹H NMR (400 MHz, d₆-DMSO) δ 9.74 (s, 1H), 8.51 (s, 1H), 8.36 (s, 1H), 7.97 (s, 1H), 7.54-7.43 (m, 1H), 7.40-7.29 (m, 1H), 7.23 (s, 1H), 6.65 (t, J=78 Hz, 1H), 4.13 (d, J=16 Hz, 2H), 3.97 (s, 3H), 3.71 (d, J=8 Hz, 2H), 2.85 (t, J=12 Hz, 2H), 1.89-1.80 (m, 1H), 1.70 (d, J=12 Hz, 2H), 1.24-1.10 (m, 2H).

### (B): N-[4-(3-chloro-2,4-difluoro-anilino)-7-methoxy-quinazolin-6-yl]-3-(difluoromethoxy)azetidine-1-carboxamide

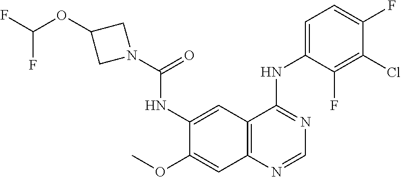 [SMILES: COc1cc2ncnc(Nc3ccc(F)c(Cl)c3F)c2cc1NC(=O)N1CC(OC(F)F)C1]

To a stirred solution of N4-(3-chloro-2,4-difluoro-phenyl)-7-methoxy-quinazoline-4,6-diamine (165 mg, 0.49 mmol) and pyridine (193 mg, 2.45 mmol) in DMF (3 mL) was added phenyl chloroformate (230 mg, 0.735 mmol) and the resulting mixture was stirred at 70° C. for 2 h. The mixture was cooled to room temperature, treated with 3-(difluoromethoxy)azetidine hydrochloride (130 mg, 0.644 mmol) and stirred at 70° C. for 2 h. The mixture was diluted with ethyl acetate and washed with brine (1×), water (1×) and brine (1×). The organic phase was dried, filtered and concentrated in vacuo then chromatographed in 50-100% ethyl acetate in hexanes. The product containing fractions were concentrated in vacuo and triturated with diethyl ether, giving the desired product as a pale orange solid (25 mg, 11%).

¹H NMR (300 MHz, d₆-DMSO) δ 9.77 (s, 1H), 8.58 (s, 1H), 8.37 (s, 1H), 7.98 (s, 1H), 7.57-7.43 (m, 1H), 7.40-7.29 (m, 1H), 7.24 (s, 1H), 6.77 (t, J=75 Hz, 1H), 5.04-4.94 (m, 1H), 4.38-4.28 (m, 2H), 4.03-3.92 (m, 2H), 3.98 (s, 3H).

Table 1a provides a summary of the LCMS characterization of the representative compounds of Formula II.

### Example 4: Biological Testing

### (A) In Vivo Efficacy in Tumor Growth in the HCC-827 Xenograft Models

Erlotinib and compounds 2A.HCl and 2D.HCl were administered to HCC-827 transformed CD1 male mice. The dosing protocol is provided in Table 2(a). Results are shown in Table 3.

### (B) Comparison of Concentrations of Compounds of the Application with Erlotinib in Brain

### Materials and Methods

### Animals

Male SD Rats were purchased from Vital River, Co. Ltd (Beijing, China). The animals were 6-8 weeks old with body weights of 200-250 g on the dosing date. The animals were housed in a 12-hour light/12-hour dark cycle environment and had free access to food and water. All animals were food fed prior to dosing. This study was approved by the Pharmaron Institutional Animal Care and Use Committee (IACUC).

### Study Design

Total 12 male SD Rats were assigned to 1 group as shown in the below. Compound 2A.HCl was administered once via oral gavage (50 mg/kg) at a dose volume of 10 mL/kg. Brain and plasma samples were collected at each time point after oral administration.

| | Dose | Dose | | | |
|---|---|---|---|---|---|
| | Level | Volume | Conc. | Administration | No. of |
| Group | (mg/kg) | (mL/kg) | (mg/mL) | Route | Animals |
| 1 | 50 | 10 | 5 | PO | 3/time point |

### Formulation Preparation

### Preparations of Dosing for PO Administration:

Added 225.73 mg of compound 2D.HCl in 42.144 mL of “0.2% CMC in 0.05% Tween-20 in water” with vortexing and sonification to obtain a suspension of 2A.HCl with concentration at 5 mg/mL.

Added 220.88 mg of compound 2A.HCl in 41.324 mL of “0.2% CMC in 0.05% Tween-20 in water” with vortexing and sonification to obtain a suspension of 2D.HCl with concentration at 5 mg/mL.

Added 230.34 mg of erlotinib in 41.324 mL of “0.2% CMC in 0.05% Tween-20 in water” with vortexing and sonification to obtain a suspension of erlotinib with concentration at 5 mg/mL.

### Sample Collection

Blood and brain samples were collected from each animal at 0.5, 1, 2 and 4 hour post-dose.

Blood samples were collected from each animal via heart puncture. These blood samples were placed into the tubes containing K2EDTA. The whole blood tubes were inverted several times and then centrifuged at 2000 g for 5 minutes at 4° C. to obtain plasma. The plasma samples were stored frozen at −75±15° C. until analysis.

Brain samples were collected after animals being fully exsanguinated. Procedure: open chest cavity, cut ventricle and perform a gentle iv saline flush (saline flush volume 20 mL) with the animal placed head down at a 45 degree angle to facilitate blood removal. The collected brain samples were washed with saline, dried with clean surgical gauze, and then put into 2 mL Eppendorf tubes and snap frozen. The brain samples were stored frozen at −75±15° C. until analysis.

### Preparation of Standard Solutions for LC-MS/MS Analysis

About 1 mg of compound 2D.HCl standard substance was weighed and dissolved in DMSO to obtain a 1 mg/mL standard stock solution in DMSO. Calibration standard working solutions were prepared at concentrations of 10, 20, 100, 500, 1000, 5000, 10000 and 20000 ng/mL by serial dilution of the standard stock solution in 50% acetonitrile. Quality control working solutions at concentrations of 30, 100, 1000, 8000 and 16000 ng/mL were prepared by serial dilution of the standard stock solution in 50% acetonitrile.

About 1 mg of the compound 2A.HCl standard substance was weighed and dissolved in DMSO to obtain a 1 mg/mL standard stock solution in DMSO. Calibration standard working solutions were prepared at concentrations of 10, 20, 100, 500, 1000, 5000, 10000 and 20000 ng/mL by serial dilution of the standard stock solution in 50% acetonitrile. Quality control working solutions at concentrations of 30, 100, 1000, 8000 and 16000 ng/mL were prepared by serial dilution of the standard stock solution in 50% acetonitrile.

About 1 mg of the erlotinib standard substance was weighed and dissolved in DMSO to obtain a 1 mg/mL standard stock solution in DMSO. Calibration standard working solutions were prepared at concentrations of 10, 20, 100, 500, 1000, 5000 and 10000 ng/mL by serial dilution of the standard stock solution in 50% acetonitrile. Quality control working solutions at concentrations of 30, 100, 1000 and 8000 ng/mL were prepared by serial dilution of the standard stock solution in 50% acetonitrile.

### Sample Treatment

All of the brain samples were diluted with water by brain weight (g) to PBS volume (mL) using a ratio of 1:3 prior to homogenizing.

5 μL of each calibration standard working solution (100, 500, 1000, 5000, 10000, 20000 ng/mL) was added to 50 μL of the blank SD rat plasma (or blank SD rat brain homogenate) to achieve calibration standards of 10-2000 ng/mL (10, 50, 100, 500, 1000, 2000 ng/mL) in a total volume of 55 μL. Quality Control (QC) samples at 10 ng/mL (low), 100 ng/mL (mid), 800 ng/mL (high-1) and 1600 ng/mL (high-2) were prepared from the QC working solutions in the same way as calibration standards. 55 μL of standards, 55 μL of QC samples and 55 μL of unknown samples (50 μL of plasma or brain homogenate with 5 μL 50% acetonitrile) were added to 200 μL of acetonitrile to precipitate proteins. Then the samples were vortexed for 30 sec. After centrifugation at 4° C., 4000 rpm for 15 min, the supernatant was diluted 2 times with water, then 10 μL of the diluted supernatant was injected into the LC-MS/MS system for quantitative analysis.

All of the samples were processed on ice.

### LC-MS/MS Conditions

The LC-MS/MS system consisted of two Shimadzu LC-30AD pumps, a DGU-20A5 degasser, a CTC Analytics HTC PAL System and an AB API4000 LC-MS/MS mass spectrometer.

Chromatographic separation was performed on a Phenomenex Luna 3μ C18 100A (30×2.00 mm) column at room temperature. The mobile phase was composed of A: 5% acetonitrile (0.1% formic acid); B: 95% acetonitrile (0.1% formic acid). The flow rate was 0.5 mL/min. The injection volume was 10 μL.

Positive mode electrospray ionization (ESI) was performed on a Turbo V® ion source to obtain a protonated ion of compounds 2A.HCl, 2B.HCl, erlotinib and Dexamethasone (IS). A multiple reaction monitoring (MRM) method was selected for quantitative analysis.

### Data Acceptance Criteria

### Acceptance Criteria of Standard Calibration Samples:

At least 6 samples should be analyzed to obtain a calibration curve. Acceptance of calibration standards requires calculated concentration within 80%-120% of the nominal concentration. 75% of the calibration standards should be within the acceptable range.

### Acceptance Criteria of Quality Control Samples:

At least 3 concentrations of quality control samples (QCs) should be analyzed in a run. Each concentration should include at least 2 individual samples. Acceptance of QCs requires calculated concentration within 80%-120% of the nominal concentration. QCs should be analyzed amongst all unknown samples and ⅔ of the QCs should be within the acceptable range, including at least 1 sample at each concentration level in an analytical run.

### Acceptance Criteria of Unknown Samples:

Unknown samples with normal peak shape of analytes and calculated concentration within the calibration range should be accepted. Samples with calculated concentration below 80% of LLOQ should be recorded as BLOQ. Samples with calculated concentration above 120% of ULOQ should be diluted with blank plasma and re-assayed. The re-assayed concentration should be multiplied by the dilution factor to obtain the final data. In cases of abnormality, such as equipment malfunction, power outage, sample treatment failure and/or sample injection failure, re-assay should be done in an individual analytical run.

### Statistical Analysis

Data acquisition was performed by Sciex Analyst 1.5.2 software (AB Sciex, Forster City, Calif.). All concentration data was reported with 3 significant figures. Data statistics were performed using Excel 2003 software. The pharmacokinetic parameters of the tested were calculated using a non-compartmental approach with Phoenix™ WinNonlin®.

### Results

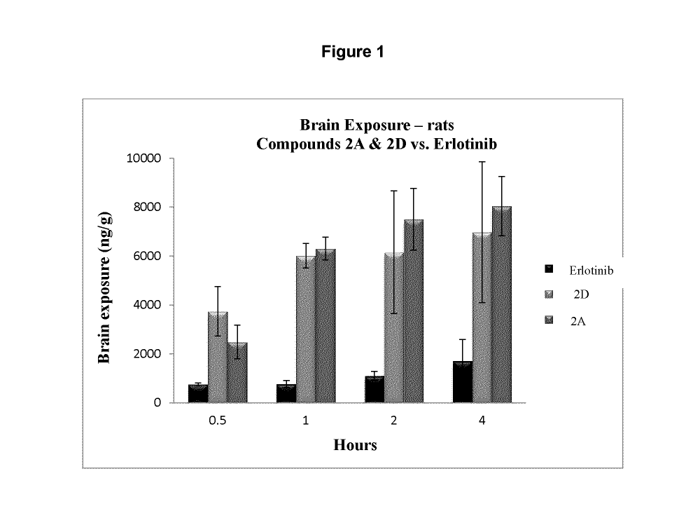

The maximum peak concentrations of erlotinib, compounds 2A and 2D were assessed in the brain tissue of 50 mg/kg rats (PO administration). Both compounds 2A and 2D had a 4× to 5× higher peak concentration in comparison to erlotinib up to 4 hours post administration (see FIG. 1, Tables 4-5).

### (C) Binding to EPHA6

For most kinase assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, and 0.5 μM non-biotinylated affinity ligand) and incubated at room temperature with shaking for 30 minutes. The kinase concentration in the eluates was measured by qPCR.

An 11-point 3-fold serial dilution of each test compound was prepared in 100% DMSO at 100× final test concentration and subsequently diluted to 1× in the assay (final DMSO concentration=1%). Most K_{d}S were determined using a compound's top concentration=30,000 nM. If the initial K_{d} determined was <0.5 nM (the lowest concentration tested), the measurement was repeated with a serial dilution starting at a lower top concentration. A K_{d} value reported as 40,000 nM indicates that the K_{d} was determined to be >30,000 nM.

### Binding Constants (K_{d}'s)

Binding constants (K_{d}'S) were calculated with a standard dose-response curve using the Hill equation:

Response=Background+Signal−Background 1+(K_{d}Hill Slope/DoseHill Slope)

The Hill Slope was set to −1.

Curves were fitted using a non-linear least square fit with the Levenberg-Marquardt algorithm.

### Results for EPHA6

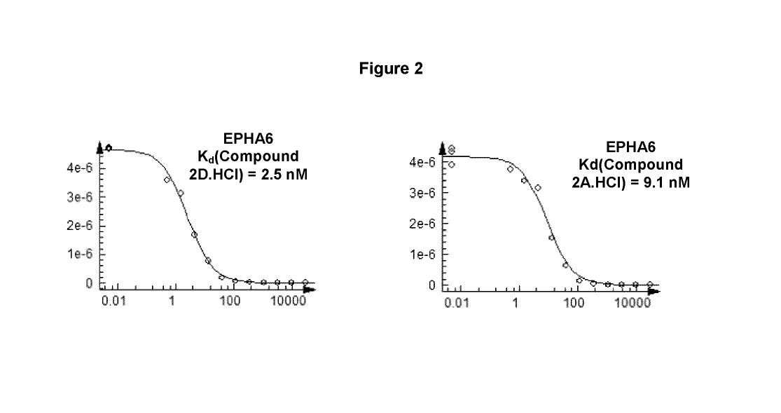

FIG. 2 shows the binding affinity values (K_{d}) of exemplary compounds 2A.HCl and 2D.HCl for the ephrin receptor kinase, EPHA6. Compounds 2A.HCl and 2D.HCl had a K_{d} of 9.1 nM and a K_{d} of 2.5 nM, respectively, Table 6 and FIG. 2.

### (D) Determination of Kinase Activity: IC₅₀

### Selectivity Against WT EGFR, Mutant EGFR and Ephrin Receptor Tyrosine Kinases

### Kinase Assays.

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.04 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Results & Discussion

Erlotinib, compounds 2A.HCl and 2D.HCl were assessed against a panel of 11 WT EGFR, mutant EGFR and ephrin receptor tyrosine kinases. Ultrasensitive quantitative PCR (qPCR) was used to measure levels of immobilized kinases after treatment with erolotinib, compounds 2A.HCl and 2D.HCl at 300 nM. All three compounds did not show selectivity against WT EGFR and mutant EGFR kinases. However, compounds 2A.HCl and 2D.HCl did show selectivity over erlotinib for the ephrin receptor kinase, EPHA6 (see Table 6).

### (E) Evaluation of P-gp Efflux

P-glycoprotein (Pgp) is a member of the ABC-transporter family that transports substances across cellular membranes acting as an energy-dependent efflux pump extruding drugs out of the cells. Increased expression of Pgp in cancer cells is one of the major mechanisms of cancer resistances and chemotherapy and thus Pgp plays a key role on the pharmacokinetics of drug absorption and distribution.

### Protocol

Human, epithelial Caco-2 cells (CRL-2102 (C2BBe1)) were seeded at a density of 40,000 cells/well, on high-density PET membrane inserts, (1.0 μm pore size, 0.31 cm² surface area) and utilized on day 21 or 22 days (post-seeding). At this stage of growth, cell monolayers were fully polarized and differentiated.

The permeability assay buffer was Hanks Balanced Salt Solution containing 10 mM HEPES and 15 mM glucose at a pH of 7.4. The dosing buffer contained 5 μM metoprolol (positive control), 5 μM atenolol (negative control) and 100 μM lucifer yellow. The buffer in the receiver chamber also contained 1% bovine serum albumin (BSA). The dosing solution concentration was 5 μM in the assay buffer. Digoxin (20 μM) was used as Pgp substrate control.

For suspected Pgp substrate, the assays were performed with and without a known Pgp inhibitor (e.g. Verapamil or Ketoconazole). The known Pgp inhibitor was co-dosed at 50 μM with compound at 5 μM.

Cell monolayers were dosed on the apical side (A-to-B) or basolateral side (B-to-A) and incubated at 37° C. in a shaker (65 rpm). Samples were taken from the donor and receiver chambers at 120 minutes. Each determination was performed in duplicate.

Narrow-window mass extraction LC/MS analysis was performed for all samples from this study using a Waters Xevo quadrupole time-of-flight (QTof) mass spectrometer, to determine relative peak areas of parent compound. The percent of transported drug was calculated based on these peak areas, relative to the initial, dosing concentration.

### Results

Results are shown in Table 7. As can be seen, compounds 2A.HCl and 2D.HCl show increased concentrations at target organs when compared to Erlotinib.

### (F) National Cancer Institute (NCI) Screening Panel

Screening of Compound 2D.HCl and Erlotinib within the NCI Panel

Compound 2D.HCl and erlotinib were screened using the National Cancer Institute (NCI) screening panel, which consists of a panel of 60 different human tumor cell lines, representing leukemia [CCRF-CEM, HL-60 (TB), K-562, MOLT-4, SR], melanoma [LOX IMVI, MALME-3M, M14, SMDA-MB-435, SK-MEL-2, SK-MEL-28, SK-MEL-5, UACC-257 and UACC-62] and cancers of the lung [A549/ATCC, EKVX, HOP-62, HOP-93, NCI-H226, NCI-H23, NCI-H322M, NCI-H460], colon [COLO 205, HCT-116, HCT-15, HT29, KM12, SW-620], brain [SF-268, SF-295, SF-539, SNB-19, SNB-75, U251], ovary [IGROV1, OVCAR-3, OVCAR-4, OVCAR-5, OVCAR-8, NCI/ADR-RES, SK-OV-3], breast [MCF7, MDA-MB-231, BT-549, T-47D, MDA-MB-468], prostate [PC-3, DU-145], and renal [786-0, A498, ACHN, CAKI-1, RXF-393, SN12C, TK-10, UO-31] cancers.

After 24 h, two plates of each cell line are fixed in situ with TCA, to represent a measurement of the cell population for each cell line at the time of drug addition (T_{z}). Experimental drugs are solubilised in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA (trichloroacetic acid). Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilised with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tᵢ), control growth, (C), and test growth in the presence of drug at the five concentration levels (Tᵢ)], the percentage growth is calculated at each of the drug concentration levels. Percentage growth inhibition is calculated as: [(Tᵢ−T_{z})/(C−T_{z})]×100 for concentrations in which Tᵢ>/=T_{z} and [(Tᵢ−T_{z})/T_{z}]×100 for concentrations in which Tᵢ<T_{z}.

Three dose response parameters are calculated for each experimental agent. Growth inhibition of 50% (GI₅₀) is calculated from [(Tᵢ-T_{z})/(C−T_{z})]×100=50, which is the drug concentration resulting in a 50% reduction in the net protein increase (as measured by SRB staining) in control cells during the drug incubation. The drug concentration resulting in total growth inhibition (TGI) is calculated from Tᵢ=T_{z}. The LC₅₀ (concentration of drug resulting in a 50% reduction in the measured protein at the end of the drug treatment as compared to that at the beginning) indicating a net loss of cells following treatment is calculated from [(Tᵢ−T_{z})/T_{z}]×100=−50. Values are calculated for each of these three parameters if the level of activity is reached. However, if the effect is not reached or is exceeded, the value for that parameter is expressed as greater or less than the maximum or minimum concentration tested.

The results obtained from this study shows compound 2D.HCl are effective against the cell lines of the 60 human tumor cell lines panel. Inhibition of human cancer cell lines in vitro by compound 2D is shown in Table 8.

### (G) Kinase HotSpot Profiling (Reaction Biology)

### Reagents:

Base Reaction buffer; 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO

*Required cofactors are added individually to each kinase reaction

### Reaction Procedure:

1. The indicated substrate was prepared in fresh Base Reaction Buffer.

2. Any required cofactors were added to the substrate solution above.

3. Indicated kinase was added into the substrate solution and gently mixed.

4. Compounds in DMSO were added into the kinase reaction mixture by Acoustic technology (Echo550; nanoliter range) and incubated for 20 minutes at room temperature.

5. 33P-ATP (specific activity 10 μCi/μl) was added into the reaction mixture to initiate the reaction.

6. The kinase reaction was incubated for 2 hours at room temperature

7. Reactions were spotted onto P81 ion exchange paper.

8. Kinase activity was detected by filter-binding method.

### Results & Discussion

Representative compounds of Formula I were evaluated against WT EGFR and mutant EGFR (L858R and L858R, T790M) kinases. IC₅₀ concentrations are illustrated in Table 9.

### (H) Human and Mouse Microsomal Stability

### Protocol

For Phase I analysis, representative compounds of the application (10 mM stock in DMSO) were incubated at a final concentration of 1 μM (this concentration assumed to be well below the Km values to ensure linear reaction conditions). Working stocks were initially diluted to a concentration of 40.0 μM in 0.1 M potassium phosphate buffer before addition to the reaction vials. Pooled mouse (CD-1, male) or human (50 donors) liver microsomes were utilized at a final concentration of 0.5 mg/ml. Duplicate wells were used for each time point (0 and 30 minutes). Reactions were carried out at 37° C. in a shaker, and the final concentration of DMSO was kept constant at 0.01%. The final volume for each reaction was 100 μL, which includes the addition of an NADPH-Regeneration solution (NRS) mix. This NRS mix is comprised of glucose 6-phosphate dehydrogenase (0.4 U/mL), NADP+ (1.3 mM), MgCl2 (3.3 mM), and glucose 6-phosphate (3.3 mM) in assay mixtures. Upon completion of the 30 minute time point, reactions were terminated by the addition of 1.5-volumes (150 μL) of ice-cold, acetonitrile with 0.5% formic acid and internal standard. Samples were then centrifuged at 4,000 rpm for 10 minutes to remove debris and precipitated protein. Approximately 150 μL of supernatant was subsequently transferred to a new 96 well microplate for LC/MS analysis.

Narrow-window mass extraction LC/MS analysis was performed for all samples using a Waters Xevo quadrupole time-of-flight (QTof) mass spectrometer and an ACQUITY UPLC system, to determine relative peak areas of parent compound.

$$\% remaining = \frac{Area count of t = 30 min}{Area count of t = 0 min} \times 100$$

### Results & Discussion

Human and mouse liver microsomes contain a wide variety of drug metabolizing enzymes and are commonly used to support in vitro ADME (absorption, distribution, metabolism and excretion) studies. These microsomes are used to examine the potential first-pass metabolism by-products of orally administered drugs. Representative compounds of the application were evaluated for their stability in human and mouse liver microsomes. A majority of the compounds of the application in both human and mouse liver microsomes were recovered within a 30 minute time period indicating that the compounds were not rapidly cleared (see Table 10).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Identification and LCMS characterization of representative
compounds of Formula I.

| Ex | | | |
|---|---|---|---|
| # | Structure | IUPAC Name | MW [M] |
| A | 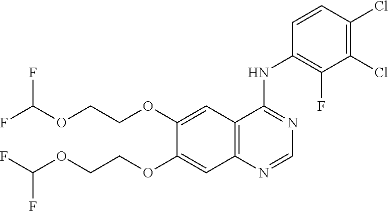 [SMILES: Fc1c(Nc2ncnc3cc(OCCOC(F)F)c(OCCOC(F)F)cc23)ccc(Cl)c1Cl] | N-(3,4-dichloro-2- fluorophenyl)-6,7-bis(2- (difluoromethoxy)ethoxy) quinazolin-4-amine | 528.26 |
| B | 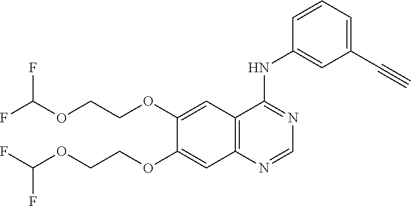 [SMILES: C#Cc1cccc(Nc2ncnc3cc(OCCOC(F)F)c(OCCOC(F)F)cc23)c1] | 6,7-bis(2- (difluoromethoxy)ethoxy)- N-(3- ethynylphenyl)quinazolin- 4-amine | 465.40 |
| C | 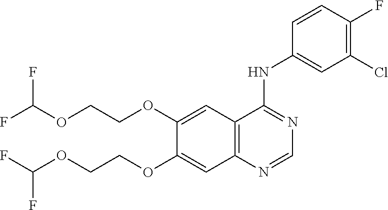 [SMILES: Fc1ccc(Nc2ncnc3cc(OCCOC(F)F)c(OCCOC(F)F)cc23)cc1Cl] | N-(3-chloro-4- fluorophenyl)-6,7-bis(2- (difluoromethoxy)ethoxy) quinazolin-4-amine | 493.81 |
| D | 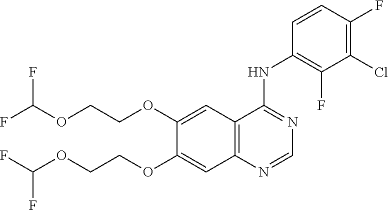 [SMILES: Fc1ccc(Nc2ncnc3cc(OCCOC(F)F)c(OCCOC(F)F)cc23)c(F)c1Cl] | N-(3-chloro-2,4- difluorophenyl)-6,7-bis(2- (difluoromethoxy)ethoxy) quinazolin-4-amine | 511.80 |
| E | 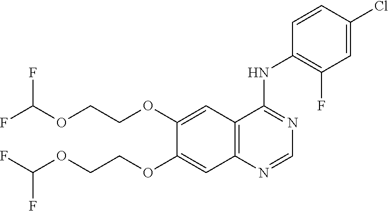 [SMILES: Fc1cc(Cl)ccc1Nc1ncnc2cc(OCCOC(F)F)c(OCCOC(F)F)cc12] | N-(4-chloro-2- fluorophenyl)-6,7-bis(2- (difluoromethoxy)ethoxy) quinazolin-4-amine | 493.81 |
| F | 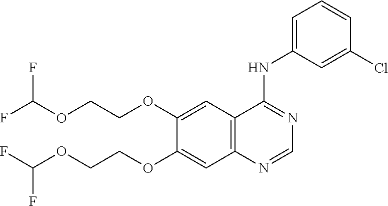 [SMILES: FC(F)OCCOc1cc2ncnc(Nc3cccc(Cl)c3)c2cc1OCCOC(F)F] | N-(3-chlorophenyl)-6,7- bis[2- (difluoromethoxy)ethoxy] quinazolin-4-amine | 475.82 |
| G | 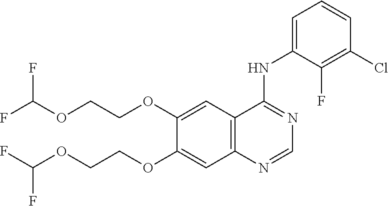 [SMILES: Fc1c(Cl)cccc1Nc1ncnc2cc(OCCOC(F)F)c(OCCOC(F)F)cc12] | N-(3-chloro-2-fluoro- phenyl)-6,7-bis[2- (difluoromethoxy)ethoxy] quinazolin-4-amine | 493.81 |
| H | 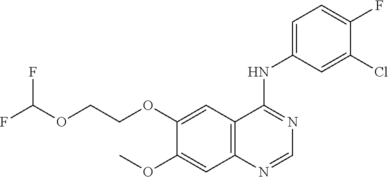 [SMILES: COc1cc2ncnc(Nc3ccc(F)c(Cl)c3)c2cc1OCCOC(F)F] | N-(3-chloro-4-fluoro- phenyl)-6-[2- (difluoromethoxy)ethoxy]- 7-methoxy-quinazolin-4- amine | 413.78 |
| I | 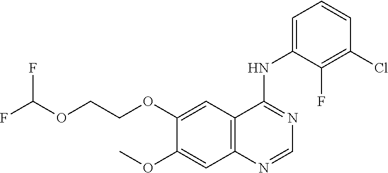 [SMILES: COc1cc2ncnc(Nc3cccc(Cl)c3F)c2cc1OCCOC(F)F] | N-(3-chloro-2-fluoro- phenyl)-6-[2- (difluoromethoxy)ethoxy]- 7-methoxy-quinazolin-4- amine | 413.78 |
| J | 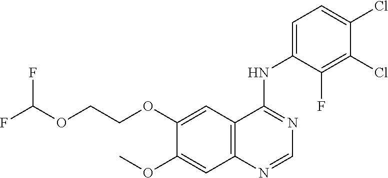 [SMILES: COc1cc2ncnc(Nc3ccc(Cl)c(Cl)c3F)c2cc1OCCOC(F)F] | N-(3,4-dichloro-2-fluoro- phenyl)-6-[2- (difluoromethoxy)ethoxy]- 7-methoxy-quinazolin-4- amine | 448.22 |
| K | 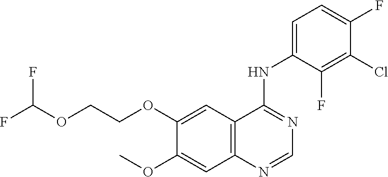 [SMILES: COc1cc2ncnc(Nc3ccc(F)c(Cl)c3F)c2cc1OCCOC(F)F] | N-(3-chloro-2,4-difluoro- phenyl)-6-[2- (difluoromethoxy)ethoxy]- 7-methoxy-quinazolin-4- amine | 431.77 |
| L | 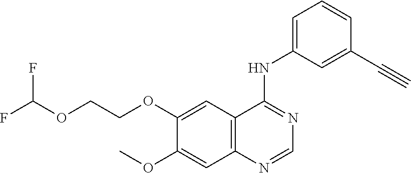 [SMILES: C#Cc1cccc(Nc2ncnc3cc(OC)c(OCCOC(F)F)cc23)c1] | 6-[2- (difluoromethoxy)ethoxy]- N-(3-ethynylphenyl)-7- methoxy-quinazolin-4- amine | 385.36 |
| M | 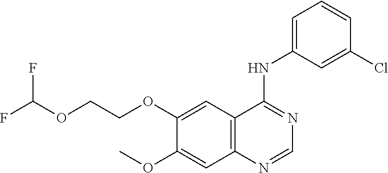 [SMILES: COc1cc2ncnc(Nc3cccc(Cl)c3)c2cc1OCCOC(F)F] | N-(3-chlorophenyl)-6-[2- (difluoromethoxy)ethoxy]- 7-methoxy-quinazolin-4- amine | 395.79 |
| N | 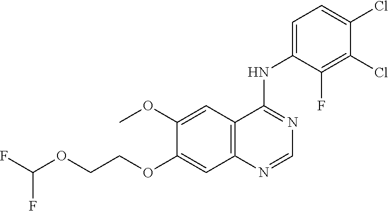 [SMILES: COc1cc2c(Nc3ccc(Cl)c(Cl)c3F)ncnc2cc1OCCOC(F)F] | N-(3,4-dichloro-2-fluoro- phenyl)-7-[2- (difluoromethoxy)ethoxy]- 6-methoxy-quinazolin-4- amine | 448.22 |
| O | 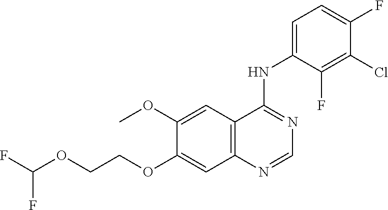 [SMILES: COc1cc2c(Nc3ccc(F)c(Cl)c3F)ncnc2cc1OCCOC(F)F] | N-(3-chloro-2,4-difluoro- phenyl)-7-[2- (difluoromethoxy)ethoxy]- 6-methoxy-quinazolin-4- amine | 431.77 |
| P | 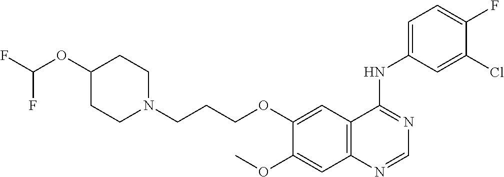 [SMILES: COc1cc2ncnc(Nc3ccc(F)c(Cl)c3)c2cc1OCCCN1CCC(OC(F)F)CC1] | N-(3-chloro-4-fluoro- phenyl)-6-[3-[4- (difluoromethoxy)-1- piperidyl]propoxy]-7- methoxy-quinazolin-4- amine | 510.94 |
| Q | 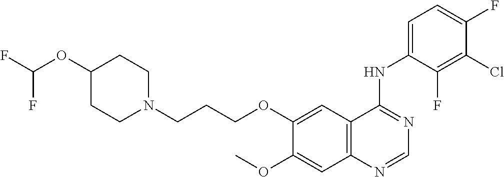 [SMILES: COc1cc2ncnc(Nc3ccc(F)c(Cl)c3F)c2cc1OCCCN1CCC(OC(F)F)CC1] | N-(3-chloro-2,4-difluoro- phenyl)-6-[3-[4- (difluoromethoxy)-1- piperidyl]propoxy]-7- methoxy-quinazolin-4- amine | 528.93 |
| R | 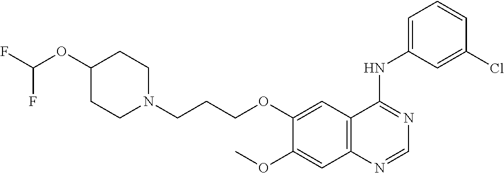 | N-(3-chlorophenyl)-6-[3- [4-(difluoromethoxy)-1- piperidyl]propoxy]-7- methoxy-quinazolin-4- amine | 492.94 |
| S | 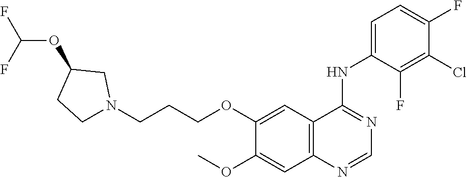 [SMILES: COc1cc2ncnc(Nc3ccc(F)c(Cl)c3F)c2cc1OCCCN1CC[C@@H](OC(F)F)C1] | N-(3-chloro-4-fluoro- phenyl)-6-[3-[(3R)-3- (difluoromethoxy)pyrrolidin- 1-yl]propoxy]-7- methoxy-quinazolin-4- amine | 496.91 |
| T | 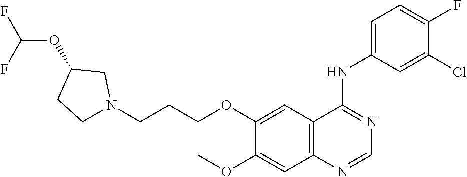 [SMILES: COc1cc2ncnc(Nc3ccc(F)c(Cl)c3)c2cc1OCCCN1CC[C@H](OC(F)F)C1] | N-(3-chloro-4-fluoro- phenyl)-6-[3-[(3S)-3- (difluoromethoxy)pyrrolidin- 1-yl]propoxy]-7- methoxy-quinazolin-4- amine | 496.91 |
| U | 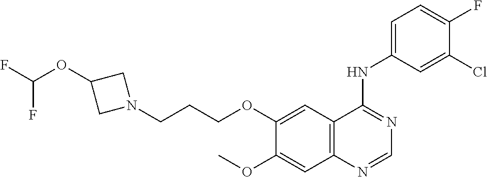 [SMILES: COc1cc2ncnc(Nc3ccc(F)c(Cl)c3)c2cc1OCCCN1CC(OC(F)F)C1] | N-(3-chloro-4-fluoro- phenyl)-6-[3-[3- (difluoromethoxy)azetidin- 1-yl]propoxy]-7-methoxy- quinazolin-4-amine | 482.88 |
| V | 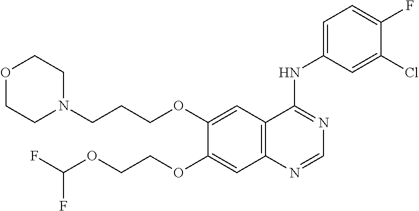 [SMILES: Fc1ccc(Nc2ncnc3cc(OCCOC(F)F)c(OCCCN4CCOCC4)cc23)cc1Cl] | N-(3-chloro-4-fluoro- phenyl)-7-[2- (difluoromethoxy)ethoxy]- 6-(3- morpholinopropoxy) quinazolin-4-amine | 526.94 |
| W | 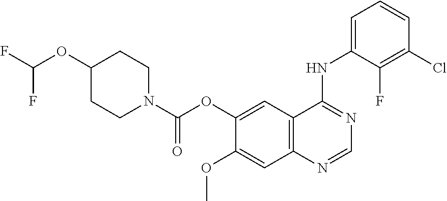 [SMILES: COc1cc2ncnc(Nc3cccc(Cl)c3F)c2cc1OC(=O)N1CCC(OC(F)F)CC1] | [4-(3-chloro-2-fluoro- anilino)-7-methoxy- quinazolin-6-yl] 4- (difluoromethoxy)piperidine- 1-carboxylate | 496.87 |
| X | 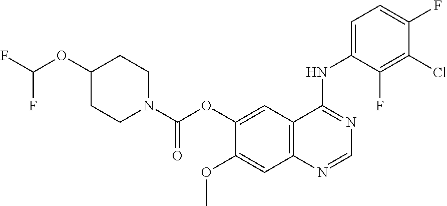 [SMILES: COc1cc2ncnc(Nc3ccc(F)c(Cl)c3F)c2cc1OC(=O)N1CCC(OC(F)F)CC1] | [4-(3-chloro-2,4-difluoro- anilino)-7-methoxy- quinazolin-6-yl] 4- (difluoromethoxy)piperidine- 1-carboxylate | 514.86 |
| Y | 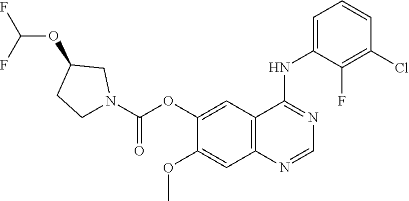 [SMILES: COc1cc2ncnc(Nc3cccc(Cl)c3F)c2cc1OC(=O)N1CC[C@@H](OC(F)F)C1] | [4-(3-chloro-2-fluoro- anilino)-7-methoxy- quinazolin-6-yl] (3R)-3- (difluoromethoxy)pyrrolidine- 1-carboxylate | 482.84 |
| Z | 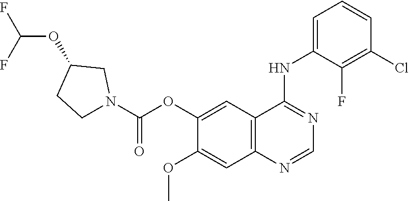 [SMILES: COc1cc2ncnc(Nc3cccc(Cl)c3F)c2cc1OC(=O)N1CC[C@H](OC(F)F)C1] | [4-(3-chloro-2-fluoro- anilino)-7-methoxy- quinazolin-6-yl] (3S)-3- (difluoromethoxy)pyrrolidine- 1-carboxylate | 482.84 |
| Aa | 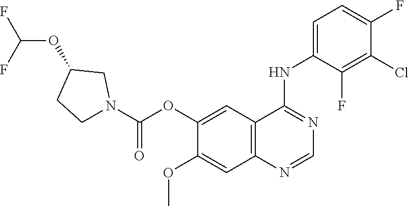 [SMILES: COc1cc2ncnc(Nc3ccc(F)c(Cl)c3F)c2cc1OC(=O)N1CC[C@H](OC(F)F)C1] | [4-(3-chloro-2,4-difluoro- anilino)-7-methoxy- quinazolin-6-yl] (3S)-3- (difluoromethoxy)pyrrolidine- 1-carboxylate | 500.83 |
| Bb | 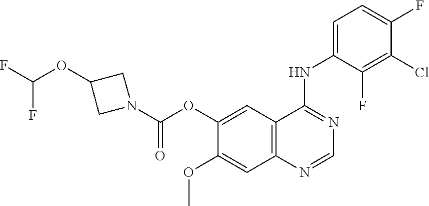 [SMILES: COc1cc2ncnc(Nc3ccc(F)c(Cl)c3F)c2cc1OC(=O)N1CC(OC(F)F)C1] | [4-(3-chloro-2,4-difluoro- anilino)-7-methoxy- quinazolin-6-yl] 3- (difluoromethoxy)azetidine- 1-carboxylate | 486.80 |
| Cc | 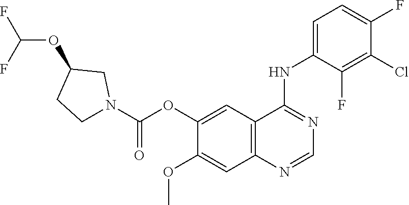 [SMILES: COc1cc2ncnc(Nc3ccc(F)c(Cl)c3F)c2cc1OC(=O)N1CC[C@@H](OC(F)F)C1] | [4-(3-chloro-2,4-difluoro- anilino)-7-methoxy- quinazolin-6-yl] (3R)-3- (difluoromethoxy)pyrrolidine- 1-carboxylate | 500.83 |
| Dd | 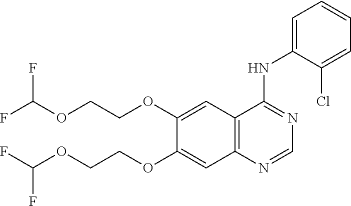 [SMILES: FC(F)OCCOc1cc2ncnc(Nc3ccccc3Cl)c2cc1OCCOC(F)F] | [4-(2-chloroanilino)-7- (2,2- dimethylpropanoyloxy) quinazolin-6-yl] 2,2- dimethylpropanoate | 475.82 |
| Ee | 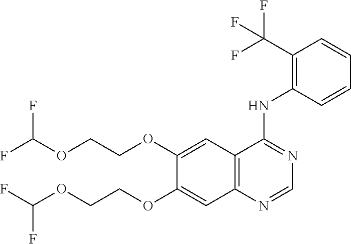 [SMILES: FC(F)OCCOc1cc2ncnc(Nc3ccccc3C(F)(F)F)c2cc1OCCOC(F)F] | 6,7-bis[2- (difluoromethoxy)ethoxy]- N-[2- (trifluoromethyl)phenyl] quinazolin-4-amine | 509.37 |
| Ff | 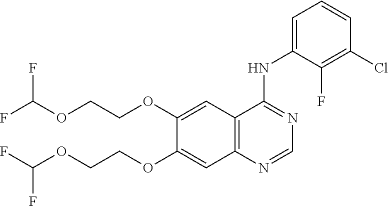 [SMILES: Fc1c(Cl)cccc1Nc1ncnc2cc(OCCOC(F)F)c(OCCOC(F)F)cc12] | N-(3-chloro-2-fluoro- phenyl)-6,7-bis[2- (difluoromethoxy)ethoxy] quinazolin-4-amine | 493.81 |
| Gg | 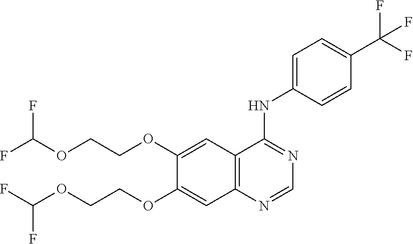 [SMILES: FC(F)OCCOc1cc2ncnc(Nc3ccc(C(F)(F)F)cc3)c2cc1OCCOC(F)F] | 6,7-bis[2- (difluoromethoxy)ethoxy]- N-[4- (trifluoromethyl)phenyl] quinazolin-4-amine | 509.37 |
| Hh | 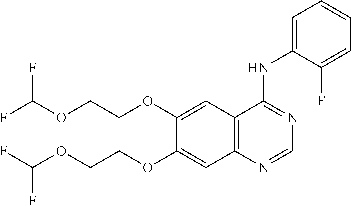 [SMILES: Fc1ccccc1Nc1ncnc2cc(OCCOC(F)F)c(OCCOC(F)F)cc12] | 6,7-bis[2- (difluoromethoxy)ethoxy]- N-(2- fluorophenyl)quinazolin- 4-amine | 459.37 |
| Ii | 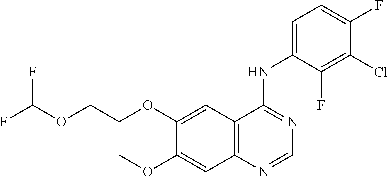 [SMILES: COc1cc2ncnc(Nc3ccc(F)c(Cl)c3F)c2cc1OCCOC(F)F] | N-(3-chloro-2,4- difluoro-phenyl)-6-[2- (difluoromethoxy)ethoxy]- 7-methoxy- quinazolin-4-amine | 431.77 |
| Jj | 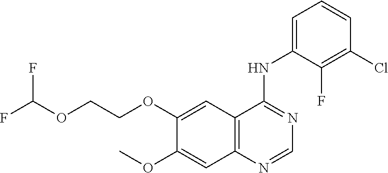 [SMILES: COc1cc2ncnc(Nc3cccc(Cl)c3F)c2cc1OCCOC(F)F] | N-(3-chloro-2-fluoro- phenyl)-6-[2- (difluoromethoxy)ethoxy]- 7-methoxy- quinazolin-4-amine | 413.78 |
| Kk | 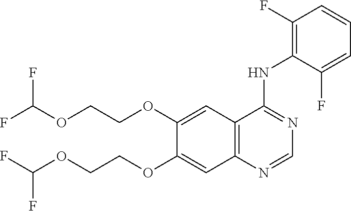 [SMILES: Fc1cccc(F)c1Nc1ncnc2cc(OCCOC(F)F)c(OCCOC(F)F)cc12] | 6,7-bis[2- (difluoromethoxy)ethoxy]- N-(2,6- difluorophenyl)quinazolin- 4-amine | 477.36 |
| Ll | 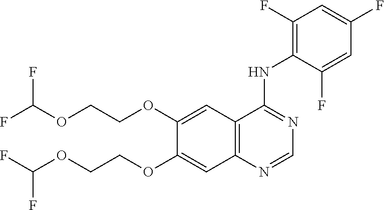 [SMILES: Fc1cc(F)c(Nc2ncnc3cc(OCCOC(F)F)c(OCCOC(F)F)cc23)c(F)c1] | 6,7-bis[2- (difluoromethoxy)ethoxy]- N-(2,4,6- trifluorophenyl)quinazolin- 4-amine | 495.35 |
| Mm | 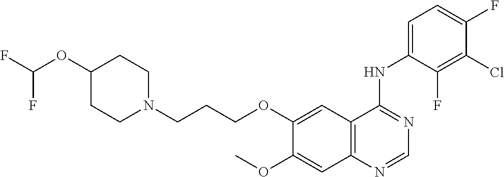 [SMILES: COc1cc2ncnc(Nc3ccc(F)c(Cl)c3F)c2cc1OCCCN1CCC(OC(F)F)CC1] | N-(3-chloro-2,4- difluoro-phenyl)-6-[3-[4- (difluoromethoxy)-1- piperidyl]propoxy]-7- methoxy-quinazolin-4- amine | 528.93 |
| Nn | 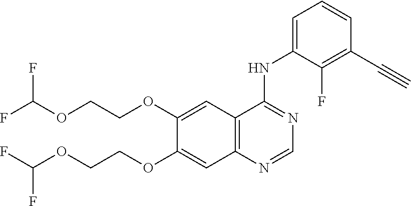 [SMILES: C#Cc1cccc(Nc2ncnc3cc(OCCOC(F)F)c(OCCOC(F)F)cc23)c1F] | 6,7-bis[2- (difluoromethoxy)ethoxy]- N-(3-ethynyl-2-fluoro- phenyl)quinazolin-4- amine | 483.39 |
| Oo | 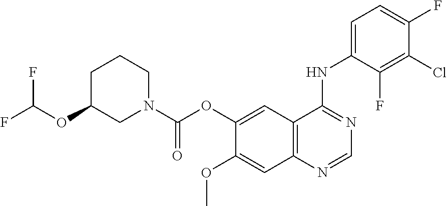 [SMILES: COc1cc2ncnc(Nc3ccc(F)c(Cl)c3F)c2cc1OC(=O)N1CCC[C@H](OC(F)F)C1] | [4-(3-chloro-2,4- difluoro-anilino)-7- methoxy-quinazolin-6- yl] (3S)-3- (difluoromethoxy) piperidine-1-carboxylate | 514.86 |
| Pp | 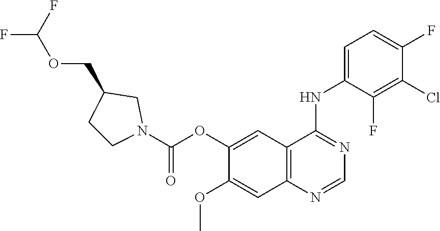 [SMILES: COc1cc2ncnc(Nc3ccc(F)c(Cl)c3F)c2cc1OC(=O)N1CC[C@@H](COC(F)F)C1] | [4-(3-chloro-2,4- difluoro-anilino)-7- methoxy-quinazolin-6- yl] (3R)-3- (difluoromethoxymethyl) pyrrolidine-1- carboxylate | 514.86 |
| Qq | 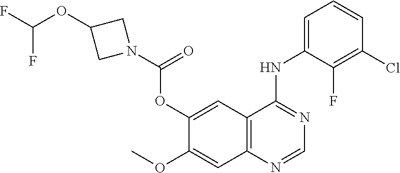 [SMILES: COc1cc2ncnc(Nc3cccc(Cl)c3F)c2cc1OC(=O)N1CC(OC(F)F)C1] | [4-(3-chloro-2-fluoro- anilino)-7-methoxy- quinazolin-6-yl] 3- (difluoromethoxy)azetidine- 1-carboxylate | |
| 3A | 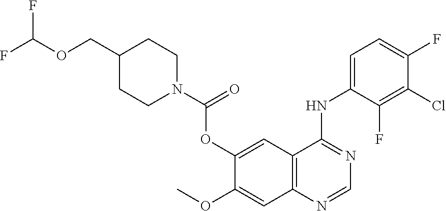 [SMILES: COc1cc2ncnc(Nc3ccc(F)c(Cl)c3F)c2cc1OC(=O)N1CCC(COC(F)F)CC1] | N-[4-(3-chloro-2,4- difluoro-anilino)-7- methoxy-quinazolin-6- yl]-4- (difluoromethoxymethyl) piperidine-1- carboxamide | 527.90 |
| 3B | 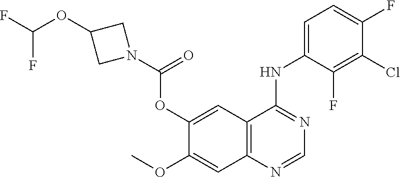 [SMILES: COc1cc2ncnc(Nc3ccc(F)c(Cl)c3F)c2cc1OC(=O)N1CC(OC(F)F)C1] | N-[4-(3-chloro-2,4- difluoro-anilino)-7- methoxy-quinazolin-6- yl]-3- (difluoromethoxy)azetidine- 1-carboxamide | 485.82 |

TABLE 2
Dosing information of Erlotinib and compounds 2A•HCl and
2D•HCl in HCC827 cell line transformed CD1 male mice.

| | | Dose | Dose | | | |
|---|---|---|---|---|---|---|
| | | Level | Volume | Conc. | Administration | |
| Group | Treatment | (mg/kg) | (mL/kg) | (mg/mL) | Route | No. of Animals |
| 1 | Erlotinib•HCl | 2 | 5 | 1 | IV | 3 M |
| 2 | | 50 | 10 | 5 | PO-A | 3 M |
| 3 | | 50 | 10 | 5 | PO-B | 2 M/time point |
| 4 | Compound | 2 | 5 | 1 | IV | 3M |
| 5 | 2D•HCl | 50 | 10 | 5 | PO-A | 3 M |
| 6 | | 50 | 10 | 5 | PO-B | 2 M/time point |
| 7 | Compound | 2 | 5 | 1 | IV | 3 M |
| 8 | 2A•HCl | 50 | 10 | 5 | PO-A | 3 M |
| 9 | | 50 | 10 | 5 | PO-B | 2 M/time point |

TABLE 3
Comparison of compounds with 1st generation
inhibitors against a lung cancer cell line.

| | Erlotinib | Gefitinib | 2A•HCl | 2D•HCl |
|---|---|---|---|---|
| Cell line | Biochemical IC₅₀ (uM) | | | |
| HCC827 | 0.046 | 0.010 | 0.021 | 0.017 |

TABLE 4
Maximum peak concentrations at 4 hours of erlotinib, compounds
2A•HCl and 2D•HCl in brain tissue of 50 mg/kg rat (PO administration).

| Route | | | | | | | AUC_{Last} |
|---|---|---|---|---|---|---|---|
| (Dosing | | Tₘₐₓ | Cₘₐₓ | AUCₗₐₛₜ | AUC_{Inf} | Cₘₐₓ Ratio | Ratio |
| Level) | Drug ID# | (hr) | (ng/g) | (hr * ng/g) | (hr * ng/g) | (Brain/Plasma) | (Brain/Plasma) |
| PO* | Erlotinib | 4 | 1713 | 4319 | N/A | 0.34 | 0.27 |
| (50 mg/kg) | Compound | 4 | 6979 | 22593 | N/A | 1.81 | 1.69 |
| | 2A•HCl | | | | | | |
| | Compound | 4 | 8040 | 25272 | N/A | 2.012 | 2.053 |
| | 2D•HCl | | | | | | |

TABLE 5
Peak concentrations at 8 hours of erlotinib, compounds 2A•HCl and
2D•HCl in brain of 50 mg/kg rat (PO administration).

| | | | | | | AUC_{Last} |
|---|---|---|---|---|---|---|
| Drug | Tₘₐₓ | Cₘₐₓ | AUCₗₐₛₜ | AUC_{Inf} | Cₘₐₓ Ratio | Ratio |
| ID# | (hr) | (ng/g) | (hr * ng/g) | (hr * ng/g) | (Brain/Plasma) | (Brain/Plasma) |
| Erlotinib•HCl | 8 | 828 | 14522 | 18449 | 0.19 | 0.18 |
| Compound | 8 | 6340 | 99996 | 101339 | 2.070 | 1.923 |
| 2D•HCl | | | | | | |
| Compound | 8 | 5606 | 101853 | 102348 | 1.718 | 1.499 |
| 2A•HCl | | | | | | |

TABLE 6
Kinome screen of erlonitinib, compounds 2A and 2D against WT
EGFR, mutant EGFR and ephrin receptor tyrosine kinases.

| DiscoveRx Gene | | FV-238•HCl | FV-240•HCl | Erlotinib |
|---|---|---|---|---|
| Symbol | Symbol | % Control @ 300 nM | | |
| EGFR(L861Q) | EGFR | 0.1 | 0 | 1.19 |
| EGFR(G719C) | EGFR | 0.2 | 0.25 | 0.28 |
| EGFR | EGFR | 0.3 | 1.2 | 0.22 |
| EGFR(L858R) | EGFR | 0.45 | 0.6 | 0.32 |
| EPHA6 | EPHA6 | 0.8** | 2.4** | 59.46* |
| EGFR(G719S) | EGFR | 0.9 | 2.3 | 0.17 |
| EGFR(L747- | EGFR | 1 | 0.45 | 0.12 |
| T751del, Sins) | | | | |
| EGFR(L747- | EGFR | 2.9 | 3.9 | 0.17 |
| E749del, A750P) | | | | |
| EGFR(S752- | EGFR | 3.2 | 2.7 | 0.53 |
| I759del) | | | | |
| EGFR(L747- | EGFR | 5.3 | 2.7 | 0.16 |
| S752del, P753S) | | | | |
| EGFR(E746- | EGFR | 6.1 | 0 | 0.16 |
| A750del) | | | | |

TABLE 7
Evaluation of erlotinib, compounds 2A and 2D as substrates for P-gp

| Compound | Verapamil | P_{app (A-B)} | P_{app (B-A)} | Efflux | Recovery (%) | |
|---|---|---|---|---|---|---|
| ID | (μM) | (10⁻⁶, cm/s) | (10⁻⁶, cm/s) | Ratio | AP-BL | BL-AP |

Control compounds:

| | | | | | | |
|---|---|---|---|---|---|---|
| Propranolol | 0 | 25.08 | 16.27 | 0.65 | 69.77 | 85.05 |
| Digoxin | 0 | 0.64 | 15.75 | 24.79 | 76.92 | 84.33 |
| Digoxin | 100 | 4.02 | 6.64 | 1.65 | 90.32 | 99.66 |

Erlotinib, Compound 2A and Compound 2D:

| | | | | | | |
|---|---|---|---|---|---|---|
| Erlotinib•HCl | 0 | 16.01 | 13.90 | 0.87 | 55.48 | 56.18 |
| Erlotinib•HCl | 100 | 27.07 | 13.36 | 0.49 | 76.82 | 58.02 |
| Compound 2D•HCl | 0 | 4.02 | 2.33 | 0.58 | 21.83 | 30.39 |
| Compound 2D•HCl | 100 | 5.64 | 3.04 | 0.54 | 29.73 | 28.91 |
| Compound 2A•HCl | 0 | 0.58 | 0.35 | 0.59 | 43.36 | 57.13 |
| Compound 2A•HCl | 100 | 1.37 | 0.70 | 0.52 | 55.50 | 83.21 |

TABLE 8
Results of a screen of compound 2D•HCl against
the NCI panel of 60 human cancer cell lines.

| | Compound 2D•HCl |
|---|---|
| NCI Panel/Cell Line | Growth % |
| Leukemia | |
| CCRF-CEM | 75.53 |
| HL-60(TB) | 80.36 |
| K-562 | 37.37 |
| MOLT-4 | 55.03 |
| RPMI-8226 | |
| SR | 75.96 |
| NSCLC | |
| A549/ATCC | |
| EKVX | 48.49 |
| HOP-62 | 64.36 |
| HOP-92 | 39.43 |
| NCI-H226 | 84.65 |
| NCI-H23 | 78.31 |
| NCI-H322M | −7.70 |
| NCI-H460 | 85.74 |
| NCI-H522 | 24.07 |
| Colon | |
| COLO 205 | 77.41 |
| HCC-2998 | 91.93 |
| HCT-116 | 81.03 |
| HCT-15 | 55.49 |
| HT29 | 58.66 |
| KM12 | 85.84 |
| SW-620 | 91.49 |
| CNS | |
| SF-268 | 70.93 |
| SF-295 | 76.73 |
| SF-539 | 73.76 |
| SNB-19 | 75.78 |
| SNB-75 | 57.99 |
| U251 | 72.65 |
| Melanoma | |
| LOX IMVI | 52.34 |
| MALME-3M | 73.73 |
| M14 | 59.47 |
| MDA-MB-435 | 56.24 |
| MDA-N | |
| SK-MEL-2 | 79.82 |
| SK-MEL-28 | 81.10 |
| SK-MEL-5 | 74.60 |
| UACC-257 | 87.43 |
| UACC-62 | 82.70 |
| Ovarian | |
| IGROV1 | 6.79 |
| OVCAR-3 | 60.72 |
| OVCAR-4 | 0.00 |
| OVCAR-5 | 26.63 |
| OVCAR-8 | 69.11 |
| NCI/ADR-RES | 54.63 |
| SK-OV-3 | −13.36 |
| Renal | |
| 786-0 | 60.48 |
| A498 | 13.11 |
| ACHN | 17.88 |
| CAKI-1 | 15.86 |
| RXF 393 | 37.95 |
| SN12C | 59.26 |
| TK-10 | 34.04 |
| UO-31 | 15.03 |
| Prostate | |
| PC-3 | 65.70 |
| DU-145 | 35.29 |
| Breast | |
| MCF7 | 66.06 |
| MDA-MB-231/ATCC | 65.62 |
| HS 578T | 68.48 |
| BT-549 | 93.32 |
| T-47D | 19.78 |
| MDA-MB-468 | −39.62 |

TABLE 9
Evaluation of the potency of representative compounds
of Formula I against WT EGFR and mutant EFGR.

| | IC₅₀ (M) | | |
|---|---|---|---|
| | | | EGFR |
| | | EGFR | (L858R, |
| Compound ID | EGFR | (L858R) | T790M) |
| 2(q) | 4.34E−11 | 1.42E−11 | 4.01E−06 |
| 2(Nn) | 5.87E−10 | 9.77E−10 | |
| 2(Oo) | 2.05E−11 | 5.20E−11 | |
| 2(Pp) | 1.43E−10 | 3.45E−10 | |
| 2(Qq) | 1.38E−11 | 1.64E−11 | 6.82E−06 |
| 3(A) | 2.98E−11 | 8.91E−11 | |
| 3(B) | 1.25E−11 | 1.38E−11 | |

TABLE 10
Representative compounds of Formula I evaluated for their
stability in human and mouse liver microsomes for 30 min.

| | MLM | HLM |
|---|---|---|
| Compound ID | (30 min) | (30 min) |
| 2(b) | 94.3 | 96.4 |
| Erlotinib | 56.3 | 75.5 |
| 2(c) | 94.6 | 88.5 |
| 2(d) | 90.4 | 83.1 |
| 2(e) | 67.8 | 89.6 |
| 2(a) | 72.5 | 90.7 |
| 2(a)•HCl | 91.7 | 97.4 |
| 2(Dd) | 22.2 | 54.7 |
| 2(Ee) | 11.8 | 49.2 |
| 2(Ff) | 69.6 | 76.4 |
| 2(Gg) | 106.2 | 99.8 |
| 2(Hh) | 51.8 | 71.6 |
| 2(k) | 0.7 | 60.4 |
| 2(i) | 1.5 | 19.7 |
| 2(Kk) | 43.7 | 77.6 |
| 2(Ll) | 39.3 | 82.1 |

1. A compound of Formula I or a pharmaceutically acceptable salt, solvate and/or prodrug thereof: 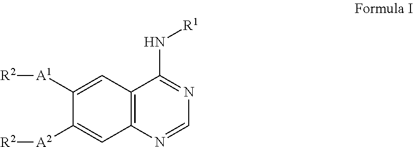
wherein:
R¹ is selected from unsubstituted or substituted aryl and unsubstituted or substituted heteroaryl, wherein the substituents for R¹ are selected from one or more of halogen, C₁₋₆alkyl, haloC₁₋₆alkyl, CN, C(O)R⁴, OR⁴, SR⁴, NR⁴R⁵, C(O)OR⁴, C(O)NR⁴R⁵, S(O)R⁴, SO₂R⁴, OC(O)R⁴, OC(O)OR⁴, OC(O)NR⁴R⁵, OC(S)NR⁴R⁵, OS(O)R⁴, OSO₂R⁴, NR⁴(OR⁵), NR⁶C(O)NR⁴R⁵, NR⁶C(S)NR⁴R⁵, NR⁵C(O)OR⁴, NR⁵C(S)OR⁴, NR⁵C(O)R⁴, C₁₋₆alkyleneC(O)R⁴, C₁₋₆alkyleneOR⁴, C₁₋₆alkyleneSR⁴, C₁₋₆alkyleneNR⁴R⁵, C₁₋₆alkyleneC(O)OR⁴, C₁₋₆alkyleneC(O)NR⁴R⁵, C₁₋₆alkyleneS(O)R⁴, C₁₋₆alkyleneSO₂R⁴, C₁₋₆alkyleneOC(O)R⁴, C₁₋₆alkyleneOC(O)OR⁴, C₁₋₆alkyleneOC(O)NR⁴R⁵, C₁₋₆alkyleneOC(S)NR⁴R⁵, C₁₋₆alkyleneOS(O)R⁴, C₁₋₆alkyleneOSO₂R⁴, C₁₋₆alkyleneNR⁴(OR⁵), C₁₋₆alkyleneNR⁶C(O)NR⁴R⁵, C₁₋₆alkyleneNR⁶C(S)NR⁴R⁵, C₁₋₆alkyleneNR⁵C(O)OR⁴, C₁₋₆alkyleneNR⁵C(S)OR⁴, C₁₋₆alkyleneNR⁵C(O)R⁴, C₂₋₆alkynyl, C₂₋₆alkynyleneC(O)R⁴, C₂₋₆alkynyleneOR⁴, C₂₋₆alkynyleneSR⁴, C₂₋₆alkynyleneNR⁴R⁵, C₂₋₆alkynyleneC(O)OR⁴, C₂₋₆alkynyleneC(O)NR⁴R⁵, C₂₋₆alkynyleneS(O)R⁴, C₂₋₆alkynyleneSO₂R⁴, C₂₋₆alkynyleneOC(O)R⁴, C₂₋₆alkynyleneOC(O)OR⁴, C₂₋₆alkynyleneOC(O)NR⁴R⁵, C₂₋₆alkynyleneOC(S)NR⁴R⁵, C₂₋₆alkynyleneOS(O)R⁴, C₂₋₆alkynyleneOSO₂R⁴, C₂₋₆alkynyleneNR⁴(OR⁵), C₂₋₆alkynyleneNR⁶C(O)NR⁴R⁵, C₂₋₆alkynyleneNR⁶C(S)NR⁴R⁵, C₂₋₆alkynyleneNR⁵C(O)OR⁴, C₂₋₆alkynyleneNR⁵C(S)OR⁴, C₂₋₆alkynyleneNR⁵C(O)R⁴ and 3-7 membered heterocycloalkyl;
R² and R³ are independently selected from C₁₋₂₀alkyl, C₆₋₂₀aryl, heteroaryl, C₃₋₂₀cycloalkyl, heterocycloalkyl, C₁₋₁₀alkyleneC₆₋₂₀aryl, C₁₋₁₀alkyleneheteroaryl, C₁₋₁₀alkyleneC₃₋₂₀cycloalkyl, C₁₋₁₀alkyleneheterocycloalkyl, C(O)C₁₋₂₀alkyl, C(O)C₆₋₂₀aryl, C(O)heteroaryl, C(O)C₃₋₂₀cycloalkyl, C(O)NR⁶heterocycloalkyl, C(O)NR⁶C₁₋₂₀alkyl, C(O)NR⁶C₆₋₂₀aryl, C(O)NR⁶heteroaryl, C(O)NR⁶C₃₋₂₀cycloalkyl and C(O)NR⁶heterocycloalkyl, wherein R² and R³ are unsubstituted or substituted with one or more substituents independently selected from halo, C₁₋₆alkyl, OC₁₋₆alkyl, halo-substituted C₁₋₆alkyl, halo-substituted OC₁₋₆alkyl, halo-substituted SC₁₋₆alkyl halo-substituted C₁₋₆alkyleneOC₁₋₆alkyl, halo-substituted C₁₋₆alkyleneSC₁₋₆alkyl, halo-substituted C₁₋₆alkyleneS(O)C₁₋₆alkyl, halo-substituted C₁₋₆alkyleneSO₂C₁₋₆alkyl and C₁₋₆alkyleneOhalo-substituted C₁₋₆alkyl, provided that at least one of R² and R³ comprises at least one fluorine atom;
R⁴, R⁵ and R⁶ are independently selected from H, C₆₋₁₀aryl, heteroaryl, C₃₋₁₀cycloalkyl, C₃₋₁₀heterocycloalkyl, haloC₁₋₆alkyl and C₁₋₆alkyl; and
A¹ and A² are independently selected from CH₂, O, S, S(O), SO₂NH and NR⁵

2. (canceled)

3. The compound of claim 1, wherein R¹ is selected from unsubstituted or substituted aryl wherein the substituents for R¹ are selected from one to four of halogen, C₁₋₆alkyl, haloC₁₋₆alkyl, CN, C(O)R⁴, OR⁴, NR⁴R⁵, C(O)OR⁴, C(O)NR⁴R⁵, C₁₋₆alkyleneC(O)R⁴, C₁₋₆alkyleneOR⁴, C₁₋₆alkyleneNR⁴R⁵, C₁₋₆alkyleneC(O)OR⁴, C₁₋₆alkyleneC(O)NR⁴R⁵, C₂₋₆alkynyl, C₂₋₆alkynyleneC(O)R⁴, C₂₋₆alkynyleneOR⁴, C₂₋₆alkynyleneNR⁴R⁵, C₂₋₆alkynyleneC(O)OR⁴, C₂₋₆alkynyleneC(O)NR⁴R⁵ and 5-6 membered heterocycloalkyl, in which R⁴ and R⁵ are independently selected from haloC₁₋₆alkyl and C₁₋₆alkyl.

4. The compound of claim 1, wherein R¹ is selected from substituted aryl wherein the substituents of R¹ are selected from one to four of Cl, F, CF₃, OR⁴, NR⁴R⁵ and C₂₋₆alkynyl in which R⁴ and R⁵ are independently selected from fluoroC₁₋₆alkyl and C₁₋₆alkyl.

5. The compound of claim 1, wherein R¹ is selected from substituted aryl wherein the substituents of R¹ are selected from one to three of Cl, F, CF₃, OR⁴, NR⁴R⁵ and C₂₋₆alkynyl in which R⁴ and R⁵ are independently selected from CF₃, CHF₂ and CH₃.

6. (canceled)

7. The compound of claim 1, wherein R¹ is selected from substituted heteroaryl wherein the substituents of R¹ are selected from one to three of Cl, F, CF₃, OR⁴, NR⁴R⁵ and C₂₋₆alkynyl and R⁴ and R⁵ are independently selected from fluoroC₁₋₆alkyl and C₁₋₆alkyl.

8. (canceled)

9. The compound of claim 1, wherein R² and R³ are independently selected from C₁₋₁₀alkyl, C₁₋₆alkyleneC₆₋₁₉aryl, C₁₋₆alkyleneC₅₋₁₀heteroaryl, C₁₋₆alkyleneC₅₋₁₀cycloalkyl, C₁₋₆alkyleneC₅₋₁₀heterocycloalkyl, C(O)C₁₋₁₀alkyl, C(O)C₆₋₁₀aryl, C(O)C₅₋₁₀heteroaryl, C(O)C₃₋₁₀cycloalkyl, C(O)NR⁶heterocycloalkyl, C(O)NR⁶C₁₋₁₀alkyl, C(O)NR⁶C₆₋₁₀aryl, C(O)NR⁶C₅₋₁₀heteroaryl, C(O)NR⁶C₃₋₁₀cycloalkyl and C(O)NR⁶C₅₋₁₀heterocycloalkyl, wherein R² and R³ are unsubstituted or substituted with one to three substituents independently selected from halo, C₁₋₆alkyl, OC₁₋₆alkyl, fluoro-substituted C₁₋₆alkyl, fluoro-substituted OC₁₋₆alkyl, fluoro-substituted SC₁₋₆alkyl fluoro-substituted C₁₋₆alkyleneOC₁₋₆alkyl, fluoro-substituted C₁₋₆alkyleneSC₁₋₆alkyl, fluoro-substituted C₁₋₆alkyleneS(O)C₁₋₆alkyl, fluoro-substituted C₁₋₆alkyleneSO₂C₁₋₆alkyl and C₁₋₆alkyleneOfluoro-substituted C₁₋₆alkyl, provided that at least one of R² and R³ comprises at least one fluorine atom.

10. The compound of claim 9, wherein R² and R³ are independently selected from: 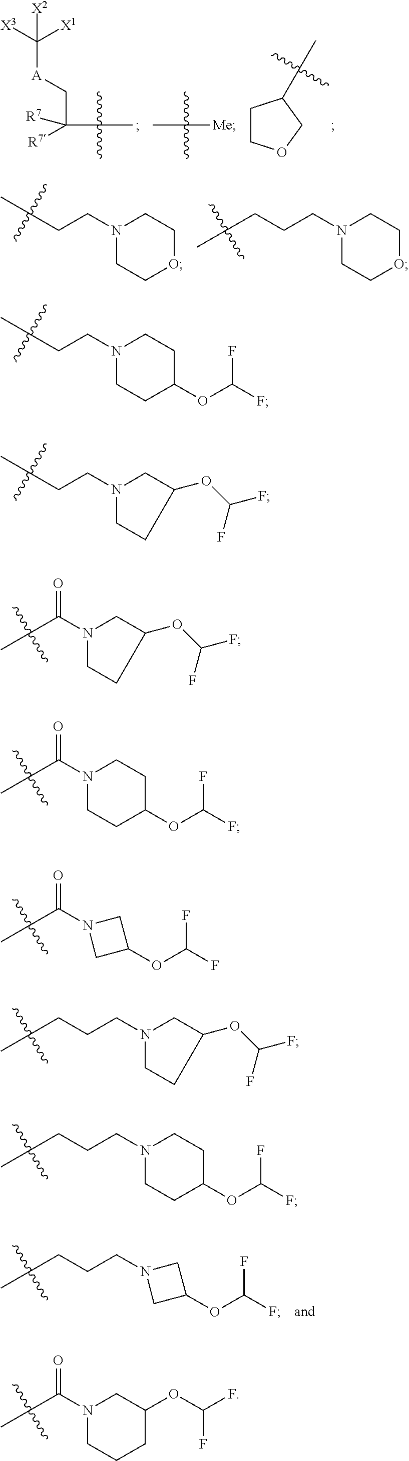
wherein R⁷ and R^{7′} are independently selected from H, aryl, heteroaryl and C₁₋₆alkyl; A is CH₂, O, S, NH or NC₁₋₆alkyl; and X¹, X² and X₃ are the same or different and are selected from H, halo and C₁₋₆alkyl.

11. The compound of claim 10, wherein R⁷ and R^{7′} are independently selected from H and C₁₋₄alkyl; A is CH₂ or O; and X¹, X² and X₃ are the same or different and are selected from H, F and C₁₋₄alkyl.

12. (canceled)

13. The compound of claim 10 wherein R² and R³ are independently selected from:

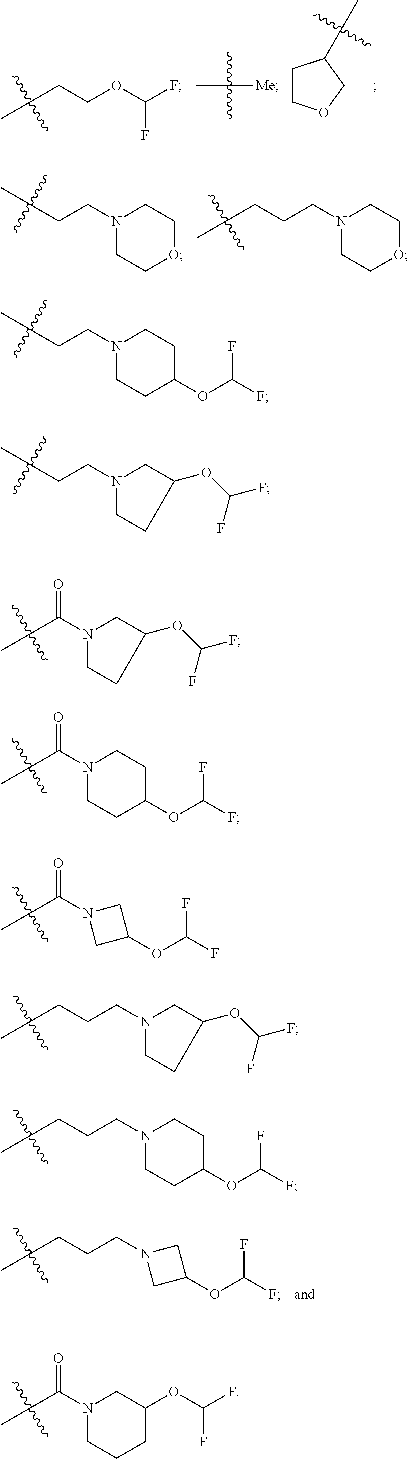

14. The compound of claim 13, wherein both of R² and R³ are F

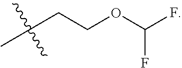 [SMILES: *CCOC(F)F]

15. The compound of claim 13, wherein one of is R² and R³ is 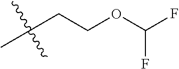 [SMILES: *CCOC(F)F]
and the other of R² and R³ is CH₃.

16. The compound of claim 1 wherein R⁴, R⁵ and R⁶ are independently selected from H, fluoroC₁₋₆alkyl and C₁₋₆alkyl.

17. (canceled)

18. The compound of claim 1, wherein A¹ and A² are independently selected from CH₂, O, NH and NCH₃.

19. (canceled)

20. The compound of claim 1, selected from:

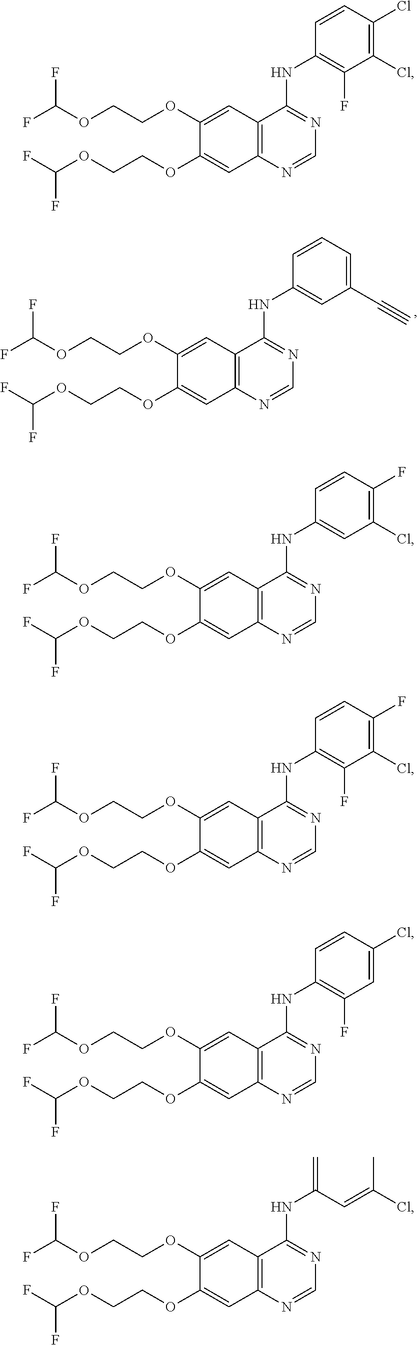 [SMILES: Fc1c(Nc2ncnc3cc(OCCOC(F)F)c(OCCOC(F)F)cc23)ccc(Cl)c1Cl ; C#Cc1cccc(Nc2ncnc3cc(OCCOC(F)F)c(OCCOC(F)F)cc23)c1 ; Fc1ccc(Nc2ncnc3cc(OCCOC(F)F)c(OCCOC(F)F)cc23)cc1Cl ; Fc1ccc(Nc2ncnc3cc(OCCOC(F)F)c(OCCOC(F)F)cc23)c(F)c1Cl ; Fc1cc(Cl)ccc1Nc1ncnc2cc(OCCOC(F)F)c(OCCOC(F)F)cc12 ; C=C(/C=C(\C)Cl)Nc1ncnc2cc(OCCOC(F)F)c(OCCOC(F)F)cc12]

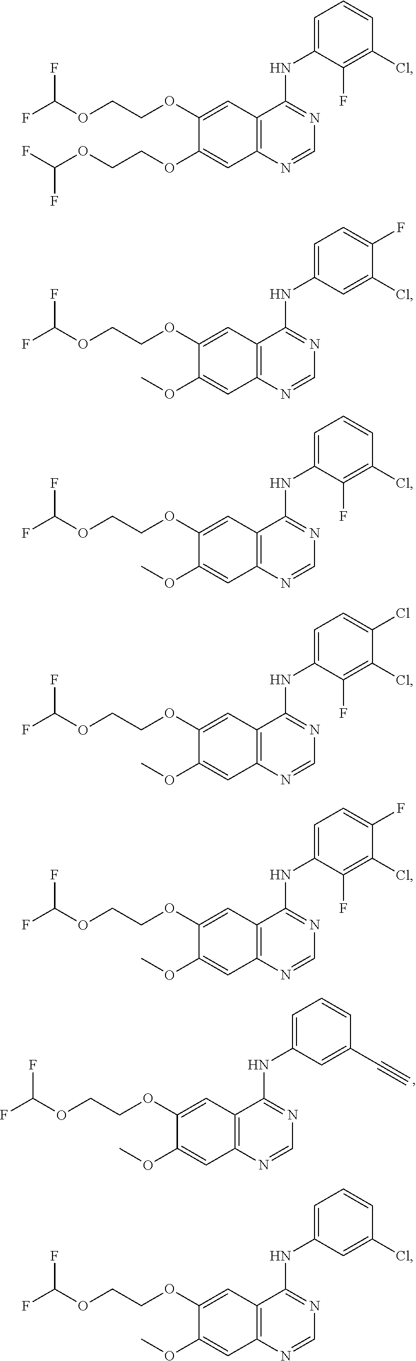

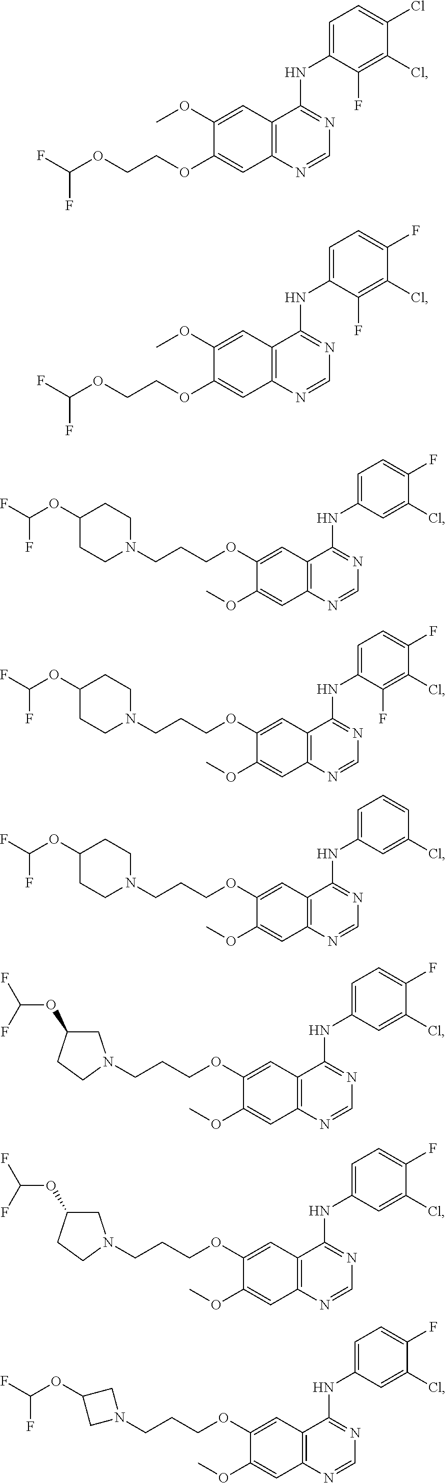

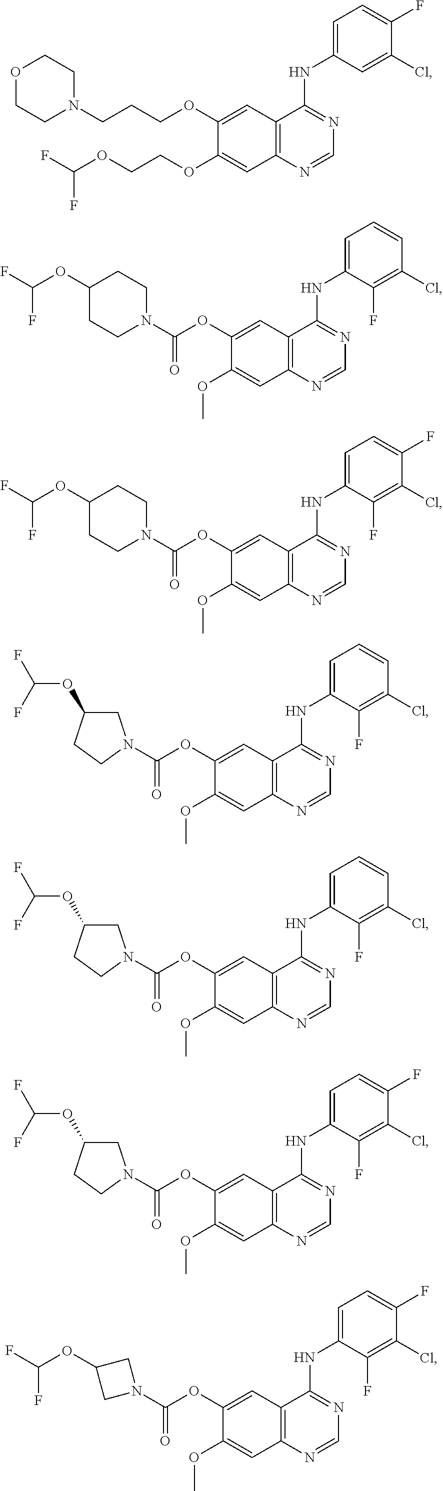

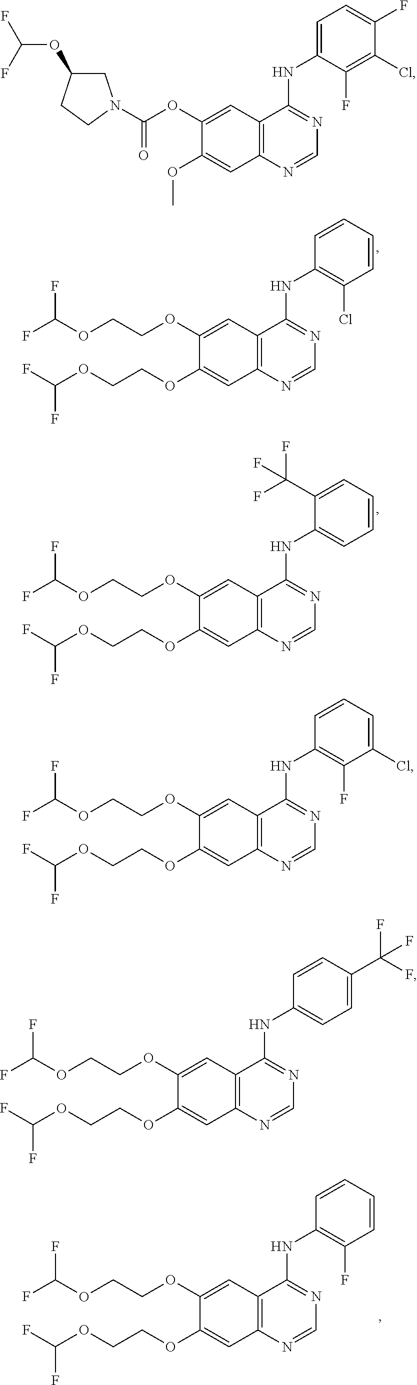 [SMILES: COc1cc2ncnc(Nc3ccc(F)c(Cl)c3F)c2cc1OC(=O)N1CC[C@@H](OC(F)F)C1 ; FC(F)OCCOc1cc2ncnc(Nc3ccccc3Cl)c2cc1OCCOC(F)F ; FC(F)OCCOc1cc2ncnc(Nc3ccccc3C(F)(F)F)c2cc1OCCOC(F)F ; Fc1c(Cl)cccc1Nc1ncnc2cc(OCCOC(F)F)c(OCCOC(F)F)cc12 ; FC(F)OCCOc1cc2ncnc(Nc3ccc(C(F)(F)F)cc3)c2cc1OCCOC(F)F ; Fc1ccccc1Nc1ncnc2cc(OCCOC(F)F)c(OCCOC(F)F)cc12]

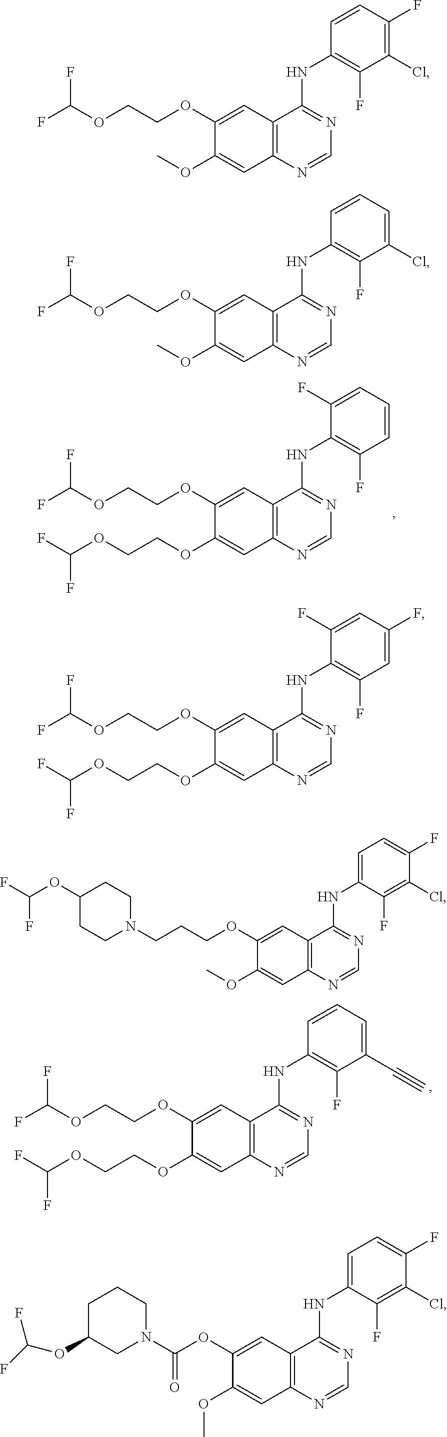

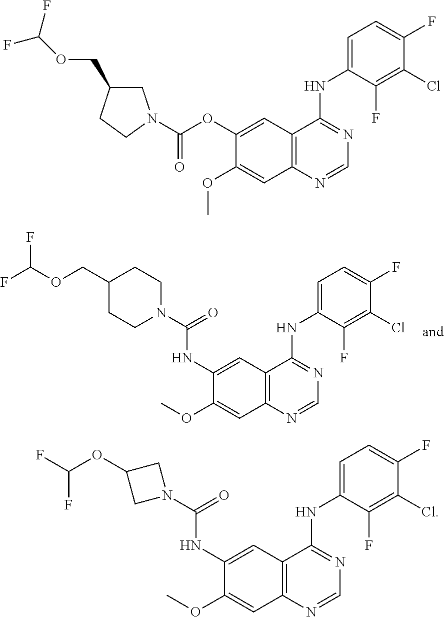 [SMILES: COc1cc2ncnc(Nc3ccc(F)c(Cl)c3F)c2cc1OC(=O)N1CC[C@@H](COC(F)F)C1 ; COc1cc2ncnc(Nc3ccc(F)c(Cl)c3F)c2cc1NC(=O)N1CCC(COC(F)F)CC1 ; COc1cc2ncnc(Nc3ccc(F)c(Cl)c3F)c2cc1NC(=O)N1CC(OC(F)F)C1]

21. The compound of claim 1 having the structure: 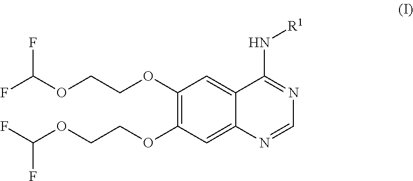
wherein R¹ is a phenyl or naphthyl group substituted with 1, 2 or 3 substituents independently selected from Cl, F, CF₃, CH₃ and C≡CH.

22. A pharmaceutical composition comprising one or more compounds of Formula (I) of claim 1, or a pharmaceutically acceptable salt, and/or solvate thereof, and a pharmaceutically acceptable carrier and/or diluent.

23. (canceled)

24. A method of treating one or more diseases, disorders or conditions mediated by EGFR comprising administering an effective amount of one or more compounds of claim 1, or a pharmaceutically acceptable salt, and/or solvate thereof, to a subject in need thereof.

25. The method of claim 24, wherein the disease, disorder or condition is a neoplastic disorder.

26. The method of claim 25, wherein the neoplastic disorder is cancer.

27. The method of claim 26, wherein the cancer is selected from breast cancer, skin cancer, prostate cancer, colon cancer, pancreatic cancer, kidney cancer, ovarian cancer, lung cancer and brain cancer.
